(19) United States
(12) Patent Application Publication
(10) Pub. No.: US 2012/0156138 A1
(43) Pub. Date: Jun. 21, 2012

(54) Methods and Compositions for the Treatment of Medical Conditions Involving Cellular Reprogramming

(76) Inventor: Larry J. Smith, Omaha, NE (US)

(21) Appl. No.: 13/264,482

(22) PCT Filed: Apr. 14, 2009
(86) PCT No.: PCT/US2009/002365

§ 371 Date: Feb. 29, 2012

(51) Int. Cl. A61K 49/00; A61K 9/127; A61K 38/00; A61K 38/08; A61K 38/10; A61K 38/16; C12N 5/02; A61P 35/00; A61P 31/14; A61P 25/28; A61P 9/10; A61P 37/02; A61P 3/10; A61P 9/00; A61P 27/02; A61P 25/00; A61P 25/16; A61P 17/06; A61P 11/06; A61P 19/02; A61P 31/12; A61P 7/06; A61P 31/18; A61P 35/02; A61K 31/7088; B82Y 5/00

(52) U.S. Cl. 424/9.2; 514/44 A; 424/450; 514/1.2; 435/375; 977/773; 977/906

(57) ABSTRACT

The present invention provides a variety of nucleic acid based therapeutics and methods of use thereof which are effective to beneficially reprogram diseased cells such that they exhibit more desirable phenotypes. Also provided are compositions and methods to reprogram normal cells for medical and commercial purposes.

### FIELD OF THE INVENTION

The present invention relates to nucleic acid based therapeutic (NABT) compositions and methods of use thereof for treating a wide variety of medical disorders. More specifically, the invention provides NABT(s) which modulate expression of biologically relevant targets, thereby ameliorating disease symptoms and associated pathology. Also provided are methods for reprogramming target cells such that they exhibit more desirable phenotypes and/or enhanced desirable functions.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

The conventional approach to drug target selection for medical conditions entails, in part, identifying those molecular targets that are directly (defined as having a direct cause-and-effect relationship with the medical condition) involved in producing the medical condition. Cancer, for example, appears to be caused by proto-oncogene activation to oncogene(s) combined with tumor suppressor gene inactivation. It follows from this conventional view, that anticancer drugs should be developed that inhibit oncogenes and/or which reinstate the activity of tumor suppressors.

In contrast, the present inventor has found that cancer, is one of a number of medical conditions where important drug targets do not have a direct cause-and-effect role to play in producing and/or in maintaining the pathologic features of the medical condition. A common aspect of these medical conditions is that they all depend on the expression of particular cellular programs for many, if not all, of their pathologic effects. These medical conditions have been termed Aberrant Programming (AP) Diseases by the present inventor and the molecular basis for such Aberrant Programming has been described in a molecular model (AP Model). This model provides important drug targets for the design of agents useful for treating such medical conditions and implicates transcriptional regulators (TRs) which control cellular programming as desirable targets. According to the AP Model, TRs are expressed by the AP cells in abnormal combinations. Thus, it is the combination of the TRs that is pathological, rather than any individual TR. In turn, this abnormal combination alters cellular programming resulting in the pathologic cellular behavior observed in these conditions. It follows from this that altering the pattern of TR expression in AP Cells is a key therapeutic goal. An unconventional aspect of this approach is that it provides that inhibiting the expression of the same TR in different cellular contexts, for example—an AP Cell verses its normal counterpart, will have different effects on cellular programming that in many instances can be exploited for therapeutic or other commercial purposes.

The AP Model also identifies AP Risk Factors for the AP disease. The presence of AP Risk Factors can lead to the occurrence of abnormal patters of TR expression. AP Risk Factors can be structurally normal or structurally abnormal molecules, including abnormal TRs or abnormally expressed TRs, and are often expressed by AP Cells. AP Risk Factors may only be important for the initiation of an abnormal pattern of TR expression or they may be needed on an ongoing basis.

The AP Model, described in U.S. Pat. No. 5,654,415 and WO 93/03770, also applies to certain medical conditions involving higher order functioning in the brain. TRs, particularly those involved in the control of cellular programming, also regulate higher-order functioning in the nervous system. NABTs directed to c-fos, for example, have been shown to alter neurological functioning in animal models (Dragunow et al., Neuroreport 5: 305, 1993). Altered patterns of TR expression in nerve cells can result in Aberrant Programming of the nerve cells, resulting in changes in patterns of neurotransmitter expression, and qualitative and quantitative changes in inter-neuronal contacts observed in certain medical conditions.

Conventional antisense oligos directed to transcripts of a given target gene vary widely in their ability to block the expression of that gene in cells. This appears to be due to 1) variations in the availability for binding of the particular target site on the transcript that is complementary to the antisense oligo; 2) the binding affinity of the oligo for the target and 3) the mechanism of antisense inhibition. Hence, what has been referred to as the poor uptake of oligos by some cell types in vitro may in large part reflect the use of antisense oligos that are not properly designed and are, therefore, not optimally potent. It is also possible that the culturing of cell lines under atmospheric oxygen conditions (which is the usual and common in vitro practice) produces a situation in which single stranded antisense oligos are made less active than they may be at much reduced (and more physiologically-relevant) oxygen tensions. The basis of this latter phenomenon could be due, at least in part, to the increased generation of reactive free oxygen radicals under ambient (atmospheric) oxygen levels by cells following treatment with any of several types of charged oligos, such as phosphorothioates. Highly reactive free oxygen radicals have been shown to have the capacity to alter the lipids in the surface membranes of cells, and to activate certain second-messenger pathways. Such alterations could lead to an inhibition of antisense oligo uptake and/or to other non-antisense oligo dependent biologic effects. A complete blockade of the induction of free radical formation by cells in response to exposure to oligos at atmospheric oxygen levels would require the presence of potent anti-oxidants such as, for example, vitamin C or vitamin E. Finally, in general, antisense oligos are more active in vitro when used on freshly obtained patient tissue specimens than they are when used on established cell lines grown (Eckstein, Expert Opin Biol Ther 7: 1021, 2007). In general, the successful treatment of cell lines in vitro with antisense oligos requires the use of a carrier. In vivo, antisense oligos are much more active compared to in vitro even if targeted to transplanted cell lines (Dean and McKay Proc. Natl. Acad. Sci. USA 91: 11762, 1994).

A significant number of the in vitro successes in the application of conventional antisense oligos for therapeutic purposes have been readily extrapolated to in vivo use. This is evidenced by the many publications showing the in vivo efficacy of antisense oligos against their intended target. Furthermore, numerous antisense oligos have been approved by regulatory agencies around the world for clinical testing. Most of these contain a phosphorothioate backbone. Pharmacologic/toxicologic studies of phosphorothioate antisense oligos have shown that they are adequately stable under in vivo conditions, and that they are readily taken up by all the tissues in the body following systemic administration (Iversen, Anticancer Drug Design 6:531, 1991; Iversen, Antisense Res. Develop. 4:43, 1994; Crooke, Ann. Rev. Pharm. Toxicol. 32: 329, 1992; Cornish et al., Pharmacol. Comm. 3: 239, 1993; Agrawal et al., Proc. Natl. Acad. Sci. USA 88: 7595, 1991; Cossum et al., J. Pharm. Exp. Therapeutics 269: 89, 1994). In addition, these compounds readily gain access to the tissue in the central nervous system in large amounts following injection into the cerebral spinal fluid (Osen-Sand et al., Nature 364: 445, 1993; Suzuki et al., Amer J. Physiol. 266: R1418, 1994; Draguno et al., Neuroreport 5: 305, 1993; Sommer et al., Neuroreport 5: 277, 1993; Heilig et al., Eur. J. Pharm. 236: 339, 1993; Chiasson et al., Eur J. Pharm. 227: 451, 1992). Phosphorothioates per se have been found to be relatively non-toxic, and the class specific adverse effects that are seen occur at higher doses and at faster infusion rates than is needed to obtain a therapeutic effect with a well chosen sequence.

Despite the numerous documented successful treatments of animal models with conventional antisense oligos, clinical successes with these molecules to date have been few. The obstacles to clinical success involve problems in the following areas: choice of animal models predictive of clinical activity, gene target choice, selection of best mechanism for inhibiting the selected gene target, selection of optimum hybridizing sequences for that purpose, proper choice of carrier to be used if any and use of interfering concomitant medications.

The present invention addresses all of these drawbacks and provides important improvements in all of these aspects, thereby providing efficacious agents for the successful treatment of a variety of different medical conditions.

### SUMMARY OF THE INVENTION

The present invention provides methods and compositions that substantially overcome a collection of impediments that together have prevented the robust use of NABTs for clinical purposes.

In one aspect, a composition, comprising in a biologically acceptable carrier, at least one nucleic acid based therapeutic (NABT) for down modulating target gene expression is provided, the NABT comprising a nucleic acid sequence which inhibits production of at least one gene product encoded by a target gene, said sequence optionally comprising one or more modifications selected from the group consisting of i) at least one modification to the phosphodiester backbone linkage; ii) at least one modification to a sugar in said nucleic acid; iii) a support; iv) at least one cellular penetrating peptide or a cellular penetrating peptide mimetic; v) an endosomal lytic moiety; vi) at least one specific binding pair member or targeting moiety; and viii) operable linkage to an expression vector, wherein said nucleic acid sequence is selected from the group of sequences in Table 8, with the proviso that when i, ii, iii, iv, v, vi, viii are absent, said nucleic acid is not SEQ ID NOS: 1, 2, 3, 4, or 2265-2293. NABTs described herein can be selected from the group consisting of an antisense NABT, a modified antisense NABT, an RNAi NABT, a modified RNAi NABT, each of the NABT optionally being encoded by an expression vector suitable for expressing said NABT in a target cell.

Table 11 provides a listing of such targets and the diseases or pathological conditions where down modulation of the targets should be effective to therapeutically reprogram cells. Table 4 provides a list of viral diseases that may be treated with the NABT described herein.

In another aspect the nucleic acid comprises at least one modified linkage or modified sugar as described further herein below. NABTs comprising piperazines, morpholinos, 2′ fluoro (e.g., fluorine in same stereo orientation as the hydroxyl in ribose), FANA and LNA modifications are particularly preferred. The NABTs encompassed by the present invention may act via a steric hindrance mechanism or they may degrade the target nucleic acid by triggering RNAse H activity. In certain embodiments, the NABT can be a gapmer which promotes RNAse H activity and exhibits increased binding affinity for the target nucleic acid.

The compositions of the invention can also comprise a support selected from the group consisting of nanoparticles, dendrimers, nanocapsules, nanolattices, microparticles, micelles, spieglemers, Hemagglutinating virus of Japan (HVJ) envelope and liposomes which facilitates uptake of the NABT into target cells.

The NABTs may optionally be linked to a cellular penetrating peptide moiety or a mimetic thereof. A variety of CPPs for this purpose are disclosed herein. Another moiety that increases the bioavailability of the NABT is an endosomal lytic component. Accordingly use of such components is also contemplated herein. To further increase specificity of targeting for the NABT, the compositions of the invention may also comprise at least one member of a specific binding pair or targeting moiety.

As mentioned above, expression vectors can be generated which comprise the NABT disclosed herein. The vector facilitates cellular uptake and expression of said NABT encoding sequences within the cell resulting in down modulation of the sequence targeted by the NABT.

In yet another embodiment, the inventive composition can be a double or single stranded siRNA molecule. Another embodiment encompasses a double stranded dicer substrate RNA comprising a passenger strand and a guide strand 25-30-nucleotides in length which is cleaved intracellularly to form substantially double stranded 21-mers with a two nucleotide (2-nt) overhang on each 3′ end. Such siRNA or dicer substrates may optionally be comprised in an expression vector.

Formulations, comprising the NABT compositions of the invention are also provided herein. Such formulations can be suitable for oral, intrabuccal, intrapulmonary, rectal, intrauterine, intratumor, intracranial, nasal, intramuscular, subcutaneous, intravascular, intrathecal, inhalable, transdermal, intradermal, intracavitary, implantable, iontophoretic, ocular, vaginal, intraarticular, otical, aerosolized, intravenous, intramuscular, systemic, parenteral, intraglandular, intraorgan, intralymphatic, implantable, slow release, and enteric coating formulations.

Also included in the present invention is a method for down modulating expression of a target gene for the treatment of an aberrant programming disease in a target cell. An exemplary method comprising administration of an effective amount of at least one composition comprising an NABT as set forth in Table 8, thereby reprogramming said target cell, said reprogramming altering the aberrant programming disease phenotype thereby providing a beneficial therapeutic or commercial effect. In certain embodiments, pairs of NABT are administered such as those pairs targeting SGP-2 or p53 as described in Tables 18-23. Such combinations can act synergistically to more effectively down modulate expression of the target sequences.

In a particularly preferred embodiment, reprogramming is therapeutically beneficial to diseased cells and normal cells are not adversely affected.

The methods for administering the NABTs of the invention can further comprise administration of an augmentation agent, selected from the group consisting of antioxidants, polyunsaturated fatty acids, chemotherapeutic agents, genome damaging agents and ionizing radiation. In particularly preferred embodiments, such agents act synergistically with the NABT described herein thereby exhibiting superior efficacy for the treatment of aberrant programming diseases. Diseases to be treated in accordance with the present invention are selected from the group consisting of Cancer, AIDS, Alzheimer's disease, Amyotrophic lateral sclerosis, Atherosclerosis, Autoimmune Diseases, Cerebellar degeneration, Cancer, Diabetes Mellitus, Glomerulonephritis, Heart Failure, Macular Degeneration, Multiple sclerosis, Myelodysplastic syndromes, Parkinson's disease, Prostatic hyperplasia, Psoriasis, Asthma, Retinal Degeneration, Retinitis pigmentosa, Rheumatoid arthritis, Rupture of atherosclerotic plaques, Systemic lupus erythematosis, Ulcerative colitis, viral infection, ischemia reperfusion injury, cardiohypertrophy, Diamond Black Fan anemia and other disorders listed in Table 11.

In yet another aspect, a method for optimizing the efficacy of NABT for treatment of aberrant programming diseases is provided. An exemplary method entails, selecting a target gene sequence which regulates cellular programming and a sequence which hybridizes therewith from Table 8, incubating the aberrantly programmed diseased cells in the presence and absence of said at least one NABT molecule, said NABT comprising one or more modifications selected from the group consisting of i) at least one modification to the phosphodiester backbone linkage; ii) at least one modification to a sugar in said nucleic acid; iii) a support; iv) at least one cellular penetrating peptide or a cellular penetrating peptide mimetic; v) an endosomal lytic moiety; vi) at least one specific binding pair member or targeting moiety; and viii) operable linkage to an expression vector. Those NABTs which exhibit improved effects on cellular reprogramming relative to cells treated NABT lacking at least one modification of these modifications is identified); thereby providing efficacious modified NABT for the treatment of aberrant programming disorders. In a further aspect, normal cells are contacted with the NABT identified, thereby identifying those NABTs which differentially affect cellular programming in aberrantly programmed cells versus normal cells. NABT to be assessed in the foregoing method can be selected from the group consisting of an antisense NABT, a modified antisense NABT, an RNAi NABT, a modified RNAi NABT, each of the NABT optionally being encoded by an expression vector suitable for expressing said NABT in a target cell.

### BRIEF DESCRIPTION OF THE DRAWINGS

FIG. 1: Graph showing Effect of NABTs targeting JunD, CREBP-1 or p53 on Acute Myelogenous Leukemic Blasts Freshly Obtained from Patients.

FIG. 2 provides schematic diagrams of many of the NABTs of the invention and the various components thereof. The most basic structure (1) is simply the sequence of the NABT per se which optionally possesses a modified backbone structure. Such molecules work via a conventional antisense mechanism, and may also depend on steric hindrance and/or RNAase H function. They can be systemically delivered and thus can target multiple affected tissue sites. In another embodiment (2), the NABT is operably linked to a cell penetrating peptide (CPP) to facilitate cellular uptake. In this construct, an endosomal lytic component may or may not be present. NABTs which function via an RNAi mechanism are shown in (3). In these constructs, the NABT is operably linked (either covalently or non-covalently) to a support molecule (e.g., a liposome or a nanoparticle), which in turn is linked to one or more CPP(s). In certain embodiments, endosomal lytic components are included in the construct to enhance intracellular delivery of the NABT. When the NABT is a conventional antisense molecule which is used for delivery to hypoxic tissues, construct (4) will be employed wherein the NABT is operably linked to a support which in turn is linked to one or more CPPs which comprise one or more endosomal lytic components. Should it be desirable to utilize NABT for delivery to hypoxic tissues which function via an RNAi mechanism, construct (5) will be employed. Such constructs comprise an RNA based NABT which is linked to a support structure which in turn is linked to one or more CPPs which comprise one or more endosomal lytic components. When specific targeting to a particular organ or tissue is desired, construct (6) can be utilized. This NABT functions via a conventional antisense mechanism and includes the NABT operably linked to a structural support which in turn is linked to at least one CPP and at least one endosomal lytic component. The construct may also comprise a receptor ligand targeting molecule to facilitate uptake of the NABT into the tissue or organ of interest. Construct (7) functions via an RNAi mechanism and is useful for facilitating delivery of the NABT to a particular organ or tissue target and comprises the NABT operably linked to a support, the support comprising one or more CPP and optionally one or more endosomal lytic components. The support may also comprise one or more receptor ligand molecules to facilitate uptake into the desired tissue. While the NABT constructs are shown in a linear fashion, the components thereof may be arranged differently provided the included components function as designed. For example, the CPP may be operably linked 5′ or 3′ to the NABT, so long as CPP and NABT activity are maintained.

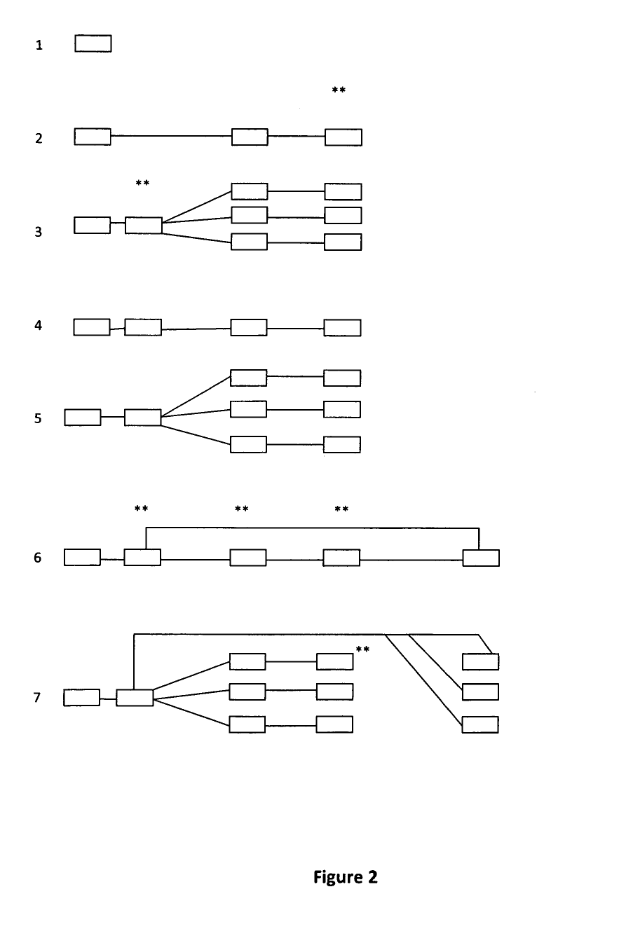

FIG. 3: A schematic diagram showing a transport moiety operably linked to the terminus of an NABT of the invention.

### DETAILED DESCRIPTION OF THE INVENTION

The present invention provides nucleic acid based therapeutics (NABTs) useful for the treatment of a wide variety of medical conditions and methods of use thereof. The NABTs of the invention may act via a conventional antisense mechanism, or RNAi mechanism and can include conventional antisense oligonucleotides (oligos), RNAi and expression vectors. The NABTs described herein are effective to modulate the expression of selected genes of interest, thereby ameliorating the pathological symptoms associated with certain medical conditions.

Methods and compositions are also provided for treating medical conditions in which the direct cause is the expression in the disordered cells (AP Cells) of one or more pathogenic cellular programs that result from the expression of abnormal combinations of transcriptional regulators (TRs). These conditions form a spectrum with those showing the most radical programming abnormalities being hereinafter referred to as Aberrant Programming (AP) Diseases. At the other end of the spectrum are Programming Disorders that have more restricted programming abnormalities. The basic molecular pathology of these medical disorders can be explained by the AP Model provided herein that in part is based on combinatorial regulation model for the control of normal cellular programming. Related embodiments provide the means for combinatorial regulation of gene expression, for reprogramming normal cells for therapeutic or other commercial purposes. The invention also relates to methods and compositions for treating AP Diseases and Programming Disorders along with a variety of other medical conditions where the target selection is based on the conventional approach of using an established cause-and-effect relationship between said molecular drug target and pathologic events that characterize the medical condition.

The following definitions and terms are provided to facilitate an understanding of the invention.

“Nucleic acid based therapeutic(s)” (NABT) are a class of therapeutic agents useful for the treatment of the medical conditions presented herein. NABTs include but are not limited to oligonucleotide and oligonucleotide-like molecules (“oligos”) that may be single or double stranded and which may be based on protein nucleic acid (PNA), RNA, DNA or other nucleotide analog chemistry defined more fully herein or a hybrid of these chemistries. NABTs include, but are not limited to, conventional antisense oligos, RNAi and expression vectors capable of causing the expression of such transcripts in cells.

“Conventional antisense oligos” are single stranded NABTs that inhibit the expression of the targeted gene by one of the following mechanisms: (1) steric hindrance—e.g., the antisense oligo interferes with some step in the sequence of events leading to gene expression resulting in protein production by directly interfering with the step. For example, the antisense oligo may bind to a region of the RNA transcript of the gene that includes a start site for translation which is most often an AUG sequence (other possibilities are GUG, UUG, CUG, AUA, ACG and CUG) and as a result of such binding the initiation of translation is inhibited; (2) induction of enzymatic digestion of the RNA transcripts of the targeted gene where the involved enzyme is not Argonaute 2. Most often the enzyme involved is RNase H. “RNase H” recognizes DNA/RNA or certain DNA analog/RNA duplexes (not all oligos that are DNA analogs will support RNase H activity) and digests the RNA adjacent to the DNA or DNA analog hybridized to it; and (3) combined steric hindrance and the capability for inducing RNA digestion in the manner just described.

NABTs that are “RNAi” make use of cellular mechanism involved in processing of endogenous RNAi. In brief, this mechanism involves the loading of an antisense oligo often referred to as a “guide strand” into a molecular complex called the RNA-induced silencing complex (“RISC”). The guide strand then directs the resultant RISC entity to its binding site on the target gene RNA transcript. Once bound, the RISC directs cleavage of the RNA target by an argonaute enzyme or in the alternative, translation may be inhibited by a steric hindrance mechanism. In a variant manifestation, the RISC may be directed to the gene itself where it can play an inhibitor function. Such NABTs may be administered in one of three forms. These are the following: (a) dicer substrates, (b) double stranded siRNA (siRNA) and (c) single stranded siRNA (ss-siRNA). With the exception of ss-siRNA, RNAi is a double stranded structure with one or more so-called passenger strand(s) hybridized to the guide strand. In most instances NABTs that are dicer substrates or that are siRNA will require a carrier to deliver them to the cytosol of the cells expressing the gene to be inhibited.

NABTs that are “expression vectors” have three basic components: (1) a double stranded gene sequence capable of driving gene expression in cells; (2) a double stranded sequence with one strand capable of giving rise to an RNA transcript that will bind to transcripts of the target gene where the sequence is oriented with respect to the sequence capable of driving expression in a way that causes this strand to be expressed in cells; and (3) a carrier capable of getting the DNA sequence just described into the nuclei of the target cells where the DNA sequence can be expressed.

For convenience, the monomers comprising the oligo sequences of individual NABTs will be termed herein “nucleotides” or “nucleosides” but it is to be understood that for NABTs, other than expression vectors, the normal sugar moiety (deoxyribose or ribose) and/or the normal base (adenine, guanine, thymine, cytosine and uracil) moieties may be substantially modified or even replaced by functionally similar analogs, for example, the normal sugar may have a fluorine inserted in the 2′ position or be entirely replaced by a different ring structure as is the case with piperazine or morpholino oligos. Further, in particular embodiments, the nucleotides or nucleosides within an oligo sequence may be abasic. In addition, the linkers between the monomers will often be varied from the normal phosphodiester structure and can include one or more of several other possibilities depending on such considerations as the need for nuclease resistance, high target sequence binding affinity, pharmacokinetics and preferential uptake by particular cell types. The alternating linker/sugar or sugar substitute structure of oligos comprising NABTs are referred to as the “backbone” while the normal bases or their substitutes occur as appendages to the backbone.

“Cell penetrating peptides” (CPPs) are peptides that promote cell penetration. CPPs may be naturally occurring protein domains or they may be designed based on the naturally occurring versions. CPPs typically share a high density of basic charges and are approximately 10-30 amino acids in length. CPPs useful in the NABTs of the invention are described further hereinbelow. “Endosomolytic and lysosomotropic agents” are agents that can be used in combination with a NABT to promote the release of said NABT from endosomes, lysosomes or phagosomes. The former are agents that are attached to NABTs or incorporated into particular NABT delivery systems while the latter agents may be so attached or incorporated or be administered as separate agents from, but in conjunction with, any such NABT used with or without a delivery system. Lysosomotropic agents have other desirable properties and can exhibit antimicrobial activity. In addition, NABTs that inhibit wild type p53 expression can interfere with endosome, lysosome and phagosome production and function thereby reducing NABT sequestration in these structures. This reduction surprisingly improves bioavailability and, therefore, enhances the inhibitory activity of NABTs that are administered during the time p53 expression is suppressed.

An endosomal lytic moiety refers to an agent which possesses at least endosomal lytic activity. In certain embodiments, an endosomal lytic moiety also exhibits lysosomolytic, phagosomolytic or lysosmotropic activity. A “specific binding pair” comprises a specific binding member and a binding partner which have a particular specificity for each other and which in normal conditions bind to each other in preference to other molecules. Such members and binding partners are also referred to as targeting molecules herein. Examples of specific binding pairs include but are not limited to ligands and receptor, antigens and antibodies, and complementary nucleic acid molecules. The skilled person is aware of many other examples. Further the term “specific binding pair” is also applicable to where either or both of the specific binding pair member and the binding partner comprise a part of a larger molecule. A “cellular program” refers to the appearance in cells, of a cell-type restricted coordinated pattern of gene expression over time. The fundamental or overarching program is a “differentiation program” that produces the basic differentiated phenotype of the cell, for example, producing a liver cell or a blood cell of a particular type, and that such differentiated phenotypes in turn determine the responses, if any, of the cell in question to exogenous or endogenous cues, for example DNA damage resulting from exposure to chemotherapy or radiation. These responses include cellular programs that control cellular viability and proliferation. Thus the differentiation program is a master program that controls various secondary programs.

A “stem cell” is a rare cell type in the body that exhibits a capacity for self-renewal. Specifically when a stem cell divides the resulting daughter cells are either committed to undergoing a particular differentiation program (along with any progeny) or they are a replica of the parent cell. In other words, the replica cells are not committed to undergo a differentiation program. When the division of a stem cell produces daughter cells that are replicas of the parent cell, the division is called “self-renewal.” Accordingly, stem cells are able to function as the cellular source material for the maintenance and/or expansion of a particular tissue or cell type.

There are many types of stem cells and often any given type exists in a hierarchy with respect to the differentiation potential of any daughter cells committed to undergoing a differentiation program. For example, a more primitive hematopoietic stem cell could have the capacity to produce committed daughter cells that in turn have the capacity to give rise to progeny that include any myelopoietic cell type while a less primitive hematopoietic stem cell might be only capable of producing committed daughter cells that can give rise to monocytes and granulocytes.

“Embryonic stem (ES) cells” are stem cells derived from embryos or fetal tissue and are known to be capable of producing daughter cells that are duplicates of the parent ES or that differentiate into cells committed to the production of cells and tissues of one of the three primary germ layers.

“Induced pluripotent (iPS) stem cells” are created (induced) from somatic cells by human manipulation. Such manipulation has typically involved the use of expression vectors to cause the expression of certain genes in the somatic cells. “Pluripotent” refers to the fact that such stem cells can produce daughter cells committed to one of several possible differentiation programs.

“Chemotherapeutic agents” are compounds that exhibit anticancer activity and/or are detrimental to a cell by causing damage to critical cellular components, particularly the genome (e.g., by causing strand breaks or other modifications to DNA). In anti-cancer applications, it may be desirable to combine administration of the NABTs described herein with administration of chemotherapeutic agents, radiation or biologics. Suitable chemotherapeutic agents for this purpose include, but are not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)).

In a particular embodiment, the chemotherapeutic agent is selected from the group consisting of: pacitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

“Biologic Agents” work by mimicking regulatory molecules including but not limited to monoclonal antibodies or antibody fragments which may be conjugated to toxins and hormone related agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

When treating prostate cancer, in addition to radiation and chemotherapeutic agents (e.g., those showing activity against prostate cancer including taxanes, anthracyclines, alkylating agents, topoismerase inhibitors and agents active on microtubules) Preferred biologic agents for use in combination with the NABTs described herein (e.g., at least one of those targeting 5 alpha-reductase, β amyloid precursor protein, cyclin A, cyclin D3, Oct-T1, p53, Pim-1, Ref-1, SAP-1, SGP2, SRF, TGF-beta), include, without limitation, the conventional androgen antagonists (such as flutamide and bicalutamide) Abarelix (an injectable gonadotropin-releasing hormone antagonist (GnRH antagonist; Plenaxis™), abiraterone acetate, an inhibitor of cytochrome p450 (17 alpha)/C17-C20 lyase; C₂₆—H₃₃—N—O₂) and Degarelix, N-acetyl-3-(naphtalen-2-yl)-D-alanyl-4-chloro-D-phenylalanyl-3-(pyridin-3-yl)-D-alanyl-L-seryl-4-((((4S)-2,6-dioxohexahydropyrimidin-4-yl)carbonyl)amino)-L-phenylalanyl-4-(carbamoylamino)-D-phenylalanyl-L-leucyl-N6-(1-methylethyl)-L-lysyl-L-prolyl-D-alaninamide, a gonadotrophin-releasing hormone (GnRH) blocker which causes significant reductions in testosterone and prostate-specific antigen (PSA) levels.

“Transcriptional regulators” (TRs) or factors are the key regulators of gene expression. TRs are well known in the art and are discussed in documents listed below: Eukaryotic Transcription Factors, D S Latchman (author), 5^{th} edition 2007, Academic Press; and Transcription Factors (Handbook of Experimental Pharmacology), M Gossen, J Kaufmann and S J Triezenberg (editors), 1^{st} edition, 2004, Springer; and Transcriptional Regulation in Eukaryotes: Concepts, Strategies, and Techniques, 2^{nd} Edition, 2009, MF Carey, C L Peterson, and S T Smale (authors), Cold Spring Harbor Press. A subset of TRs can act together to control cellular programming by operating as a combinatorial regulation system. See Table 1. In other words, cellular programs are controlled by particular combinations of TRs rather than by individual TRs. Further, more than one such combination of TRs can produce basically the same effect on cellular programming. Consequently, a particular TR capable of being involved in cellular programming may or may not be necessary for the occurrence of a particular program depending on what other TRs are being expressed as well as on certain other factors such as the availability of particular genes for being expressed. Thus, the functional consequences of the expression of any given TR are context dependent.

In addition to cellular programming, TRs control the expression of housekeeping genes and/or genes whose expression is associated with a particular cellular phenotype such as hemoglobin expression in red blood cells. Any given TR may be restricted to the regulation of one of these groups of genes to the exclusion of the others or it may be involved in the regulation of multiple types of genes as just described but not necessarily at the same time.

There are estimated to be between 20,000 to 50,000 genes in the human genome distributed over 3×10⁹ base pairs of DNA. In any given cell type approximately 10,000 genes are expressed. Greater than 90% of these are expressed by many cell types and the large majority of these are referred to as “housekeeping genes.” Typically, differentially expressed genes in any given cell type number in the hundreds. It is these genes that make the difference between liver cells and brain cells, for example. The large majority of these are directly involved in carrying out the functions that characterize the cell type. Liver cells, for example, express a wide range of enzymes that are involved in ridding the body of many types of chemicals. Thus, given the modest number of non-housekeeping genes to be regulated in any given cell type and the power of combinatorial regulation systems to control complex phenomenon with few regulatory elements, it follows the number of TRs and their direct modifiers that are needed to control cellular programming in any given cell type is small.

Thus, although Table 1 presents a fairly long list of TRs involved in cellular programming, it should be understood that only a few TRs will be expressed by any given cell type. Accordingly, sub-combinations of suitable NABTs selected based on the medical condition to be treated should exhibit efficacy for the treatment of that medical condition. Of the TRs involved in cellular programming, certain TRs are more broadly expressed by various tissue types than others. These include but are not limited to the following: p53, AP-1, c-myc, Ets-1, Ets-2, NF-kappaB, E2F-1, ID-1, Oct-1, Rb and YY-1. Examples of TRs involved in cellular programming known in the art to have very restricted expression patterns include but are not limited to androgen receptor, estrogen receptor, the numerous hox TRs, HB24, HB9, EVX-1, EVX-2, L-myc, N-myc, OTF-3 and SCL. It is thus possible to prioritize the TRs listed in Table 1 based on their use in particular cell types and their particular pattern of TR expression.

Further, TRs often occur in families so that single probes can be designed that will facilitate detection of multiple TR encoding nucleic acids in simultaneous screening assays. An example is a single homeobox probe for screening for the presence in any given cell type of any of the multiple homobox genes. Other TR families appearing in Table 1 that can be screened for as groups, include, but are not limited to the following families: ATF, C/EBP, myc, jun, fos, myb, Ets, E2F, Gata, ID, IRF, MAD, Oct and SP. More restricted probes can then be used to further discriminate particular TRs in cells shown to express at least one member of a particular TR family using a more general probe. Thus, targeted cell types can be efficiently and rapidly screened for their pattern of TR expression.

The specific TRs and direct modifiers involved in regulating cellular programming expressed by a given cell type have either been previously determined or can be readily determined by the use of a variety of well established techniques several of which are presented herein.

TRs bind to other TRs and in certain cases also bind to an enhancer or silencer. The result of such binding is that the associated TR group or groups collectively associated with all the enhancers and silencers associated with a given transcribed sequence of DNA controls the levels of transcription of the associated DNA. Such transcribed DNA may be a gene (encoding a protein) or it may encode regulatory RNA such as microRNA.

TRs may be either normal or mutated and/or be expressed at normal or abnormal levels. According to the AP Model, an essential aspect of these medical conditions is the expression in the AP Cells of qualitatively and/or quantitatively abnormal combinations of TRs, where the TRs are among those involved in the control of cellular programming (Table 1) e.g., differentiation, proliferation and apoptosis. TRs may undergo alternative splicing or post-translational modifications that fundamentally alter their function. The molecular mechanisms that produce such modifications in TRs are varied and molecules producing such modifications are referred to herein as “direct modifiers”. Direct modifiers are also suitable targets for the practice of the present invention. Table 2 provides a list of relevant TRs and Table 3 includes a listing of the direct modifiers of these TRs. Such direct modifiers include but are not limited to certain tyrosine kinases.

Targeting of TRs or their direct modifiers for purposes other than altering cellular programming can find therapeutic use in accordance with the present invention. This approach hinges on a conventional cause and effect role for the TR in the pathology of the medical condition and does not necessarily hinge on the AP Model.

“Combinatorial regulation” refers to a regulation system for complex phenomenon determined by the expression pattern of different components acting in concert rather than on the expression of any given component. Perhaps the most common examples of a combinatorial system are alphabet-based languages where the letters in the alphabet are the regulatory components. Some of the embodiments of the present invention are based on combinatorial regulation models for the control of cellular programming, as provided herein, where the key components of the regulation system are TRs.

Several investigators have proposed that combinatorial regulation plays a general role in eukaryotic gene expression. See Scherrer, and Marcand, J Cell Phys 72: 181, 1968; Sherrer, Adv Exp Med Biol 44: 169, 1924; Gierer, Cold Spring Harbor Symp Quant Biol 38: 951, 1973; Stubblefield, J Theor Biol 118: 129, 1986; Bodnar, J Theor Biol 132: 479, 1988; and Lin and Riggs, Cell 4: 107, 1975. Using biophysical arguments, these authors demonstrated the impossibility of having a separate regulator for every gene in a eukaryotic cell. Combinatorial regulation models of eukaryotic gene expression generally postulate multiple levels of regulation in addition to transcription. In principle, these models show theoretically how 100,000 genes could be selectively controlled by as few as 50 regulatory molecules, only a small subset of which would operate at the level of what is defined here as a TR(s). Bodnar, J Theor Biol 132: 479, 1988.

As in language where the alphabet can generate words with the same effect (synonyms) the TR components of the key controlling mechanism for cellular programming can be grouped in a multiplicity of ways that produce basically the same impact on cellular programming. Accordingly, different TR patterns of expression can be expected between AP Diseases and Programming Disorders compared to their normal counterparts and between different types of normal cells. This situation provides the basis for a specificity of biologic effect and, therefore, therapeutic effect for NABTs and other drugs that affect TR expression and/or function.

It should be clear that the range of reasonable therapeutic drug targets for the treatment of a particular medical disorder where the targets function as part of a combinatorial regulation system is different than the range of reasonable targets based on the conventional approach to rational drug development. The latter is based on the establishment of simple consistent “cause-and-effect relationships” across a variety of cell types between the functions of a particular target molecule and a pathologic feature(s) of a particular medical disorder. The expression of the target molecule in question does not in all instances mean the effect will be seen but it does mean that if said target molecule produces a given effect of this nature, that the effect will be consistent. For example, bcl-2 functions to inhibit programmed cell death across a variety of cell types. This has been established on a simple cause-and-effect basis. Depending on what other bcl family members are expressed, however, bcl-2 expression in a given cell may or may not successfully inhibit programmed cell death in a particular situation, such as the occurrence of DNA damage to the cell in question, but importantly bcl-2 never functions to promote programmed cell death. Thus, in this context, bcl-2 is an example of a cell program regulator that does not function as part of a combinatorial regulation system.

A major embodiment of the present invention relates to methods and compositions for treating “Aberrant Programming (AP) Diseases” and “Programming Disorders.” These medical conditions include but are not limited to those listed in Table 2. These medical conditions share a common molecular pathology described by the “AP Model” in which the “direct cause” is the expression in the disordered cells that characterize said condition (“AP Cells”), of one or more cellular programs that are abnormally expressed and/or functionally abnormal. These abnormalities require the expression of abnormal (qualitative and/or quantitative abnormalities) combinations of TRs that operate as part of a combinatorial regulation system to control cellular programming. A salient feature of combinatorial regulation systems is that they require very few components in order to control very much larger and more complex systems. In other words, AP Diseases and Programming Disorders are directly caused by the expression of qualitative and/or quantitative combinations of TRs that do not occur in normal cells.

The cellular programs involved in these medical conditions include cellular differentiation, proliferation and viability (programmed cell death, senescence, autophagy, mitotic catastrophe, programmed necrotic cell death as well as other cellular programs for disabling cells—(For simplicity these programs will all be referred to as “apoptosis” in the following text although this term is usually restricted to programmed cell death. This is appropriate in this context because all of these cell disabling mechanisms are controlled by the same basic molecular mechanism involving TRs and described by the AP Model and thus, are cellular behaviors which can be targeted with the therapeutic approach, and NABTs set forth herein.)

The term “direct cause” with respect to pathogenesis of an AP Diseases or Programming Disorders which are characterized by abnormal patterns of TR expression is to be conceptually distinguished from the presence of “AP Risk Factors” although in some instances there will be an overlap where a particular AP Risk Factor has a direct causal role by both being responsible for producing an abnormal pattern of TR expression (the direct cause) and by also being a member of that abnormal pattern. In such an instance, the AP Risk Factor is structurally normal. Patterns of TR expression and, therefore, aberrant cellular programs can evolve over time and the expression of an abnormal pattern of TRs can become independent of any AP Risk Factors that were involved in producing the original defect.

Typically an AP Disease or Programming Disorder, and many other medical conditions, will be associated with “causal factors” that in various combinations may appear to “cause” or at least promote the likelihood of the medical condition. Often such risk factors are found on the basis of a statistically significant correlation. These risk factors can be, but are not limited to, the occurrence of abnormally expressed molecules where the abnormality is qualitative, as in a mutation, and/or quantitative. Such causal factors are to be distinguished from AP Risk Factors as defined herein.

In addition to identified specific molecular changes “AP Risk Factors” as well as “causal factors” more generally may, but not necessarily include, mutagenic events, viral infections, chromosomal abnormalities, genetic inheritance, improper diet and psychological state. The field of epidemiology provides the means for identifying both AP Risk Factors and causal factors. (Modern Epidemiology, K J Rotman, S Greenland and T L Lash, (2008) 3^{rd} edition, Lippincott Williams & Wilkins, New York, N.Y.)

AP Diseases and Programming Disorders can be manifested as a metaplastic, hyperplastic or hypoplastic condition or a combination of these. Certain molecular AP Risk Factors, such as mutated and/or over-expressed proteins, can be useful targets for the treatment of AP Diseases or Programming Disorder. These are a subset of “Molecular Risk Factors” a term that is more generally applied herein. As just stated, “Molecular Risk Factors” can be identified without the insights provided by the AP Model where normal genes encoding TRs or their direct modifiers become legitimate targets for therapeutic intervention as a result of their functioning as part of a combinatorial regulation system. Accordingly, “Molecular Risk Factors” also may be useful targets for treating a variety of medical conditions that include more that just AP Diseases and Programming Disorders, but in these instances they are identified by epidemiologic-like methods and do not require the AP Model for their identification.

It follows from these observations that cells with a particular differentiated phenotype can be “differentially reprogrammed” compared to other cells with a different differentiated phenotype by altering the expression of a single TR that may be expressed by both cell types. So differential reprogramming can involve inhibiting the expression of the same target in two different cell types and getting a different effect on cellular programming when the two cell types are compared. This applies to both normal cells and to AP cells.

The capacity of a particular NABT or combination of NABTs to cause differential reprogramming is generally but not necessarily determined by the “Reprogramming Test” disclosed herein. The Reprogramming Test can initially be carried out in vitro but it may also be carried on in vivo. In the case of AP Diseases and Programming Disorders, it is applicable both to potential targets selected on the basis of the AP Model and to targets that are selected based on the establishment of cause-and-effect relationships and where said targets are known modulators of apoptosis. Such targets, with bcl-2 being an example, may be modulators of cellular programming but the nature of their effect on cellular programming is not determined by their being part of a combinatorial regulation system. Targets of this nature are suitable for the practice of the present invention as provided for herein.

“dsRNA” refers to a ribonucleic acid based NABT molecule having a duplex structure comprising two anti-parallel nucleic acid strands with sufficient complementarity between adjacent bases on opposite strands to have a Tm of greater than 37° C. under physiologic salt conditions. dsRNA that are delivered as drugs typically will have modifications to the normal RNA structure and/or be protected by a carrier to provide stability in biologic fluids and other desirable pharmacologic features as described in more detail herein. The RNA strands may have the same or a different number of nucleotides.

“Introducing into” means uptake or absorption in the cell, as is understood by those skilled in the art. Absorption or uptake of NABTs can occur through cellular processes, or via the use of auxiliary agents or devices.

As used herein and as known in the art, the term “identity” is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., SIAM J. Applied Math. (1988) 48:1073. “Substantially identical,” as used herein, means there is a very high degree of homology preferably >90% sequence identity.

As used herein, the term “treatment” refers to the application or administration of a NABT or other therapeutic agent to a patient, or application or administration of a NABT or other drug to an isolated tissue or cell line from a patient, who has a medical condition, e.g., a disease or disorder, a symptom of disease, or a predisposition toward a disease, with the purpose to cure, heal, alleviate, relieve, alter, remedy, ameliorate, improve, or affect the disease, the symptoms of disease, or the predisposition toward disease. In an alternative embodiment, tissues or cells or cell lines from a normal donor may also be “treated”.

As used herein, a “pharmaceutical composition” comprises a pharmacologically effective amount of a NABT, optionally other drug(s), and a pharmaceutically acceptable carrier. As used herein, “pharmacologically effective amount,” “therapeutically effective amount” or simply “effective amount” refers to that amount of an agent effective to produce a commercially viable pharmacological, therapeutic, preventive or other commercial result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier or diluent for administration of a therapeutic agent. Pharmaceutically acceptable carriers for therapeutic use are well known in the pharmaceutical art, and are described, for example, in Remington's Pharmaceutical Sciences, A R Gennaro (editor), 18^{th} edition, 1990, Mack Publishing, which is hereby incorporated by reference herein. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Two strategies for rationally identifying groups of drug targets were employed for the present invention. One of these is based on the AP Model and includes drug targets that comprise TRs involved in the control of cellular programming and their direct modifiers, Table 3. The other strategy is based on the establishment of direct cause-and-effect relationships and it applies to other medical disorders as well as to AP Diseases and Programming Disorders as well as to normal cell reprogramming. An important subgroup of such cause-and-effect relationships involve medical conditions where some or all of the pathologic features of the disorder result from the expression or lack of expression of an apoptosis program. Table 4 provides a list of such conditions with the AP Diseases and Programming Disorders listed at the top (4A) and other medical disorders listed at the bottom (4B). Table 5 provides a list of reasonable targets for these disorders that are not TRs and that are established on the cause-and-effect basis. This list included p53 because it can directly function in the regulation of apoptosis programs independently of its TR function. The initial selection of particular gene targets and the associated NABTs for such medical conditions are shown in Tables 2 and 4. In the case of the medical conditions shown in Table 4 the effect a successful NABT will exhibit on the AP Cells is provided in Table 6A or on damaged normal cells in Table 6B.

Individuals skilled in the art are well aware that several of the medical conditions listed in Table 2 as AP Diseases or Programming Disorders present clinically with more than one mechanistic basis, for example, type 1 and type 2 diabetes mellitus. In type 1, the underlying pathology is associated with the loss of the cells that produce insulin. In type 2, the underlying pathology results from the resistance of target cells for insulin to insulin. It follows that the application of the AP Model to AP Diseases and Programming Disorders with differences in the underlying pathology will likely respond to treatments targeting different therapeutic agents. Some conditions, such as obesity, will include subsets of patients with an underlying pathology that is obviously not related to alterations in cellular programming. In the case of obesity, the NABTs are designed to target molecules which function in cellular programming in the patient's adipocytes or are targeted to TRs exhibiting abnormal TR expression in these cells. Certain forms of obesity result from aberrant cellular programming in a patients adipocytes. Thus, NABT which target and reprogram the cells to reduce the increased deposit of fat are particularly preferred for this purpose. The specific cellular programs, TRs and their direct modifiers to be targeted are provided herein.

In some instances, the NABTs of the present invention will achieve the intended therapeutic goal more effectively when used in combination with an “augmentation agent.” Augmentation agents include anticancer treatments, agents causing oxidative stress or oxidative damage to cells (including but not limited to free-radical generators), antioxidants and agents that modulate TR expression and/or function. Guidance is provided herein on which augmentation agents are apt to be useful for particular purposes. Also discussed are situations where the agents do not function as augmentation agents, but on the contrary are contraindicated for use with particular NABTs and/or in the treatment of certain medical conditions. In addition to medications that are apt to be given to the patients of interest for NABT treatment, it is also important to consider what nutraceuticals patients are apt to be taking independent of and during administration of prescribed NABT containing regimens. The potential usefulness of an augmentation agent for use in combination with an NABT intended to alter cellular programming can be determined by means of the Reprogramming Test as applied in vitro and/or in vivo. A well established example in the art of the use of NABTs with augmentation agents is the use of conventional antisense oligos directed to targets that resist apoptosis in combination with anticancer treatments to treat cancer.

A free-radical generator could be used as an “augmentation agent” in combination with an antisense NABT designed in accordance with the present invention particularly in diseases where the objective is to kill the AP cell (for example, atherosclerosis, or cancer). Free radical generators include, but are not limited to, certain polyunsaturated fatty acids (including gamma linolenic acid, eicosapentaenoate and arachidonate), chemotherapeutic agents and ionizing irradiation as well as certain novel anticancer agents in development such as, but not limited to, inhibitors of oxygen radical scavengers as well as the reactive oxygen species (ROS) generators TDZD-8 (4-benzyl-2-methyl-1,2,4-thiadiazolidine-3,5-dione, a glycogen synthase kinase 3 inhibitor) and elesclomol.

Antioxidants have multiple potential effects that can impact the efficacy of a variety of therapeutic agents including but not limited to NABTs. These effects depend on the dose, NABT and medical condition being treated. Such effects include the induction of cell cycle arrest, induction of or inhibition of apoptosis, altering TR expression and/or function (e.g., NF-kappaB) as well as to scavaging free radicals, thereby limiting the biologic effects of the NABT.

Antioxidants include, but are not limited to, certain vitamins, minerals, trace elements and flavinoids. A complete listing of antioxidants would include those known to those skilled in the art, and may be found in standard advanced textbooks, such as, Zubay G L: “Biochemistry” (3rd edition), in 3 Volumes, Wm C Brown Communications, Inc., 1993; and in: Rice-Evans C A and Burdon R H (eds): “Free Radical Damage and Its Control”, New York: Elsevier, 1994; and in: Yagi K et al (eds): “5th International Congress on Oxygen Radicals and Antioxidants”, New York: Excerpta Medica Press, 1992 (International Congress Series, No. 998). Anti-oxidants that have been used clinically include, but are not limited to: ascorbic acid (vitamin C), allopurinol, alpha- and gamma-tocopherol (vitamin E), beta-carotene, N-acetyl cysteine, Desferol, Emoxipin, glutathione, histidine, lazaroids, Lycopene, mannitol, and 4-amino-5-imidazole-carboxamide-phosphate.

Information relating to the impact of particular oxidants and/or antioxidants on cells generally or in particular medical conditions is available in the art and can be found in the following documents: Alzheimer Disease: Neuropsychology and Pharmacology, G Emilien, C Durlach, K L Minaker, B Winblad, S Gauthier and Jean-Marie Maloteaux (Authors) Birkhäuser Basel; 1st edition, 2004; Oxidative Stress and the Molecular Biology of Antioxidant Defenses, JG Scandalios (Editor) Cold Spring Harbor Laboratory Press; 1st edition, 1996; Free Radicals and Inflammation, PG Winyard, DR Blake and CH Evans (Editors) Birkhäuser Basel; 1st edition, 2000; Oxygen/Nitrogen Radicals: Cell Injury and Disease, V Vallyathan, V Castranova and X Shi (Authors) Springer; 1 edition, 2002; Free Radicals, Oxidative Stress, and Antioxidants: Pathological and Physiological Significance, T Özben (Editor) Springer; 1st edition, 1998; Nutrients and Cell Signaling (Oxidative Stress and Disease), J Zempleni and K Dakshinamurti (Editors) CRC; 1st edition, 2005; Phytochemicals in Health and Disease (Oxidative Stress and Disease), Y Bao and R Fenwick (Editors) CRC; 1st edition, 2004; Natural Compounds in Cancer Therapy: Promising Nontoxic Antitumor Agents From Plants & Other Natural Sources, J Boik (Author) Oregon Medical Press; 1st edition, 2001; Handbook of Antioxidants (Oxidative Stress and Disease), L Packer and E Cadenas (Authors) CRC; 2nd edition, 2001; Anticancer Therapeutics, S Missailidis (Author) Wiley; 1st edition, 2009; Handbook of Nutrition and Food, CD Berdanier (Editor) 1st edition, 2001; Signal Transduction by Reactive Oxygen and Nitrogen Species: Pathways and Chemical Principles, HJ Forman, JM Fukuto and M Torres (Editors) Springer; 1st edition, 2003; Oxidative Stress and Neurodegenerative Disorders, G A Qureshi (Author), GAl Qureshi; SH Parvez (Editors) Elsevier Science; 1st edition, 2007; Oxidative Stress and Inflammatory Mechanisms in Obesity, Diabetes, and the Metabolic Syndrome, L Packer and H Sies (Editors) CRC; 1st edition, 2007; Macular Degeneration, PL Penfold and JM Provis (Editors) Springer; 1st edition, 2004; Oxidants in Biology: A Question of Balance, G Valacchi and PA Davis (Editors) Springer; 1st edition, 2008; Nutrient-Gene Interactions in Cancer, S Choi and S Friso (Editors) CRC; 1st edition, 2006; Nutrient-Gene Interactions in Health and Disease, N Moustaid-Moussa and CD Berdanier (Editors) CRC; 2nd edition, 2001; Endothelial Dysfunctions and Vascular Disease, R De Caterina and P Libby (Editors) Wiley-Blackwell; 1st edition, 2007; Nutrition and Wound Healing, JA Molnar (Editor) CRC; 1st edition, 2006; Antioxidants and Cardiovascular Disease, R Nath (Author), M Khullar (Author), PK Singal (Editor) Alpha Science International, Ltd, 2004; Cerebral Vasospasm, B Weir and RL Macdonald (Authors) Academic Press; 1st edition, 2001; Free Radical and Antioxidant Protocols, D Armstrong (Editor) Humana Press; 1st edition, 1998; Oxygen Radicals and the Disease Process, C Thomas (Author) CRC; 1st edition, 1998; Redox Biochemistry, R Banerjee, D Becker, M Dickman, V Gladyshev and S Ragsdale (Editors) Wiley; 1st edition, 2007; Free-Radical-Induced DNA Damage and Its Repair: A Chemical Perspective, C von Sonntag (Author) Springer; 1st edition, 2006.

The principal effects of free-radical generators or anti-oxidants on cells from the perspective of the AP Model is to produce an alteration in the pattern of TR being expressed, or, in the case of antioxidants, to prevent the adverse effects on cells produced by cellularly-generated free radicals subsequent to NABT binding. It follows from the AP Model that this pattern will be different following treatment with these “augmentation agents” when normal cells are compared with AP Cells. Hence, it is possible to combine this treatment with a pre-determined antisense NABT selected according to the criteria given herein (for example, in the Reprogramming Test) and expect different results for normal versus AP Cells.

The TRs that are known to be involved in cellular responses to free-radicals and apoptosis include, but are not limited to: the AP-1 group, including junD; the Egr group; Gadd group; Hox group; IRF group; the MAD, Max and Mxi groups; myc and myb groups; NF-kappaB; p53; Ref-1; Sp-1; TR-3 and TR-4; and USF. Other genes include those directly involved in the regulation of apoptosis that are not TRs. See Table 5.

“Hotspots” have been identified for more than 200 gene targets which are indexed in Table 7 and listed by sequence (provided in Table 8). Hotspots are continuous antisense sequences of varying lengths that form a template for oligos that are surprisingly well suited for use in NABTs where the NABT has at least one such strand that recognizes a gene or RNA transcript by complementary base or base analog pairing. Such NABTs tend to exhibit higher activity and fewer side effects than those chosen by the methods previously described in the art.

In the case of NABTs that are RNAi, this reduction in side effects includes a reduction in the inhibition of microRNA processing by cells and the concomitant reduction in the adverse effects of interfering with normal microRNA function. For each hotspot, one or more typically shorter sequences were selected to serve as prototype NABTs where the NABT is a conventional antisense oligo although they can be adapted for RNAi use. Size variant oligos suitable for use in conventional antisense and RNAi are also provided in Table 8. In the case of NABTs that are RNAi (dicer substrates or siRNA either single stranded or double stranded) certain modifications to the prototype sequence or size variants may be preferable in accordance with the guidance provided herein for the selection of optimal RNAi NABTs. NABTs based on the sequences provided in Table 8 can be used to study the functions of the genes they target as well as for other commercial uses and medical indications as described herein.

For the purposes of initial in vitro NABT screening and/or for commercial in vitro NABT use, carriers will typically be needed, particularly for RNAi. For conventional antisense oligos, cationic liposomal carriers have long been used for in vitro purposes and alternatively operably linked cell penetrating peptides (CPPs) may be employed. More complex carriers are more commonly used with RNAi for both in vitro and for in vivo use. For most in vivo use involving NABTs that are conventional antisense oligos or single stranded siRNA, a carrier will not be necessary. Preferred carriers suitable for use in the present invention are provided in more detail elsewhere herein.

In certain instances, NABTs which are effective to modulate target gene expression will be further assessed under a variety of different experimental conditions. Testing initially will be carried out in vitro but may be initially carried out in vivo particularly in situations where there is no suitable culture system for the AP Cells or in the case of the development of NABTs for medical conditions involving higher order brain functioning such as psychosis, depression or epilepsy. In other instances, the Reprogramming Test described herein can be applied to a significant degree in vivo. Methods for monitoring cell proliferation in vivo are well established and include methods based on immunohistochemistry and/or on metabolic labeling procedures. Further, in the last 10 years numerous techniques have been developed for the non-invasive monitoring of apoptosis in vivo. These techniques include but are not limited to those based on PET, SPECT, MRI, MRS, ultrasound and real-time imaging. These techniques are discussed in numerous documents including but not limited to the following: Kenis et al., Cell Mol Life Sci 64: 2859, 2007; Lahorte et al., Eur J Nucl Med Mol Imaging 31: 887, 2004; Corsten et al., Curr Opin Biotechnol 18: 83, 2007; Schoenberger et al., Curr Med Chem 15: 187, 2008; Flotats et al., Eur J Nucl Med Mol Imaging 30: 615, 2003; Blankenberg, Curr Pharm Des 10: 1457, 2004; and Belhocine and Blankenberg, Curr Clin Pharmacol 1: 129, 2006.

An NABT designed to inhibit the expression of a particular gene in human cells may not have an identical oligo sequence(s) to an NABT designed to inhibit the same gene in animal cells. Thus, in certain cases, the species specific homolog of an NABT may be synthesized in order to further characterization of the capacity of the NABT to reprogram cells in a therapeutically beneficial manner. Oligos for use in NABTs directed to animal versions of the gene targets listed in Table 7 can be obtained using the method described herein that was used to generate the oligo sequences for the human NABTs. In many instances, the animal oligo sequence will be derived from the human sequence by correcting any mismatches and then testing to see if the design criteria are still met. If not, an alternative animal oligo sequence can readily be generated using the design principles provided herein. Should animal cells need to be cultured to test NABTs directed to genes expressed by non-human cells, many references describing such culture systems are available to those with ordinary skill in the art and include but are not limited to the following: Animal Cell Culture Methods, L Wilson (Author) Academic Press; 1 edition, 1998 and Animal Cell Biotechnology: Methods and Protocols, R Pörtner (Editor) Humana Press; 2nd edition, 2007. The backbone chemistries and other design issues for such animal NABTs will follow the same principles provided herein for NABTs directed to human gene targets. Obviously, xenotransplantion of the appropriate human cells into an animal model can help mitigate the need for separate testing of an animal and a human version of a NABT directed to a particular gene target.

In cases where it is desirable to further assess or optimize NABT function, (e.g., cases where it is desirable to assess the effects of alteration of the carrier, backbone structure, and/or attached CPP for example) any in vivo testing initially will involve animal models, but in some instances initial efficacy testing will occur in patients following selection of an NABT capable of effectively inhibiting the desired gene target after appropriate pharmacokinetic and toxicologic testing is performed. The latter would occur in instances where suitable in vitro or animal models are not available. This could occur for reasons that include the following: (1) the AP cells from patents cannot be grown in vitro for a sufficient length of time to carry out NABT testing; (2) there is no available cell line with a phenotype that closely resembles the AP Cells in patients; (3) the available animal models do not show the key pathogenic features of the disorder in question in patients; (4) the AP Cells that may be used in otherwise apparently suitable in vitro or animal models do not have a TR expression pattern (Table 1) that is very similar to what is seen in the AP Cells from patients; or (5) the AP Cells otherwise appropriate for the in vitro or animal models fail to express a non-TR apoptosis regulator (Table 5) of interest. In vitro and in vivo models applicable to the development of the commercial uses for the NABTs provided herein are provided in Tables 9 and 10.

In another embodiment, NABTs containing nucleic acid sequences selected from Table 8 where said sequences are complementary to portions of RNA transcripts of target genes selected from Tables 3 or 5 and where the genes are expressed by the target cells are used to reprogram normal cells. Such normal cell reprogramming includes but is not limited to performing the following either in vitro or in vivo: (1) generating iPS cells from various somatic starting cell types such as, but not limited to, brain-derived neural stem cells, neural crest stem cells, keratinocytes, hair follicle stem cells, fibroblasts, hepatocytes and hematopoietic cells (Lowry and Plath Nature Biotech 26: 1246, 2008; Aasen et al., Nature Biotech 26: 1276, 2008; Silva et al. PLOS Biology 6: e253, 2008; Mali et al., Stem Cells 26: 1998, 2008; Lowry et al., Proc Natl Acad Sci USA 105: 2883, 2008; Dimos et al., Science 321: 1218, 2008). In a preferred embodiment, iPS cells to be used for tissue repair and engineering are prepared from somatic cells taken from the patient for whom said tissue repair is to be undertaken; (2) maintaining and expanding ES cells including ES cell lines; and (3) directing the differentiation of iPS or ES cells including ES cell lines into desired cell types such as but not limited to nerve, cardiac, skin or islet cells for tissue repair and engineering. Such ES and iPS cells can be used for a variety of medical purposes including but not limited to tissue repair and engineering, fighting infection or treating autoimmune diseases. It is often desirable to expand iPS or ES cell numbers and/or maintain them in a state where they can be readily reprogrammed to express a particular differentiated phenotype. NABTs of the invention can be used to advantage to prevent iPS or ES cell senescence and to promote stem cell proliferation. Target genes for such an application include but are not limited to p53, Rb, NF-kappa B, Waf-1, AP-1 and certain other gene targets associated with stem cell proliferation and differentiation as listed in Table 11 where the applications include reprogramming normal stem cells (Zeng, Stem Cell Rev 3: 270, 2007). In the case where the NABT to be used for these purposes is an expression vector, it is preferred that the vector not integrate into the host genome. Vectors of this type are well known in the art and documents describing them include but are not limited to the following: Stadtfeld et al., Science 322: 945, 2008; Ren et al., Stem Cells 24: 1338, 2006; and Paz et al., Hum Gene Ther 18: 614, 2007. In the case of conventional antisense oligonucleotides, those combined with cell penetrating peptides such as the arginine-rich peptides described herein, are preferred particularly for treating stem cells propagated in vitro and most particularly for stem cell lines that are being propagated in vitro. This approach avoids the toxic effects of cationic liposomal carriers and facilitates the use of uncharged antisense oligonucleotides such as those with a morpholino replacement of the normal sugar wherein the nucleosides are joined by phosphorodiamidate linkage(s).

Commercial applications of stem cells along with methods of culturing, tissue engineering and administration for therapeutic purposes are described in the following references: Stem Cell Therapy and Tissue Engineering for Cardiovascular Repair: From Basic Research to Clinical Applications, N Dib, DA Taylor and EB Diethrich (Editors) Springer; 1 edition 2005; Cell Therapy, Stem Cells and Brain Repair, CD Davis and PR Sanberg (Editors) Humana Press; 1 edition 2006; Hematopoietic Stem Cell Therapy, JW Lister, P Law and ED Ball (Editors) Churchill Livingstone, 2000; Stem Cell Therapy for Autoimmune Disease, AM Marmont and RK Burt (Editors) Landes Bioscience; 1 edition 2004; Stem Cell Therapy, EV Greer (Editor) Nova Biomedical Books; 1 edition, 2006; Vodyanik and Slukvin, Curr Protoc Cell Biol, Chapter 23: Unit 23.6, 2007; Desbordes et al., Cell Stem Cell 2: 602, 2008; Wang et al., Blood 105: 4598, 2005; Zhang et al., Stem Cells 24: 2669, 2006; Yao et al., Proc Natl Acad Sci USA 103: 6907, 2006; Peura et al., Theriogenology 67: 32, 2007; Skottman et al., Regenerative Med 2: 265, 2007; Trounson, Ernst Schering Res Found Workshop 54: 27, 2005; Vodyanik and Slukvin, Curr Protoc Cell Biol, Chapter 23: Unit 23.6, 2007; Vodyanik and Slukvin, Meth Mol Biol 407: 275, 2007; Principles of Tissue Engineering, Second Edition, RP Lanza, R Langer and JP Vacanti (Authors) Academic Press; 2 edition, 2000.

In other embodiments, it may be desirable to reprogram normal cells such that they exhibit improved biological functions or phenotypes. Additional examples of normal cell reprogramming include but are not limited to the following: (1) expanding the population of hematopoietic stem cells to treat medical conditions associated with blood cell deficiencies; (2) expanding cell numbers of some tissue or cell type prior to transplant or to produce increased quantities of cellularly produced molecular products for commercial use.

Therapeutically relevant cells engineered to have clinically improved phenotypes using the NABTs of the invention can be obtained from the patient to be treated and then may be employed for transplantation of the cells back into the individual (autologous transplant). In an alternative approach, cells may be obtained from another donor (allogeneic transplant) engineered using the NABT described herein and reintroduced into the individual in need of treatment. This embodiment comprises the steps of:
a) obtaining therapeutically relevant cells from the individual (or donor) and
b) exposing the therapeutically relevant cells to a reprogramming amount of an NABT capable of altering the expression and/or function of a TR and administering the treated cells to an individual.

The “Reprogramming Test” will be performed where appropriate to assess combinations and or modifications of the NABTs provided herein. Target gene expression will be assessed in the cells of interest, and the cells contacted with structural variants of the NABTs showing promise to determine their ability to ameliorate symptoms of the medical condition to be treated.

Desirable reprogramming changes in AP Cells treated with NABTs that inhibit the target genes shown in Table 3 include the following: (1) death or senescence of the AP cells; or (2) a stable change in the phenotype of the AP Cells such that some or all of the pathologic features of the AP Cells are lost. Reprogramming changes in AP Cells treated with NABTs that inhibit the targets shown in Table 5 should produce either a promotion or an inhibition of apoptosis depending on the target. The desired effect will depend on the AP Disease or Programming Disorder to be treated and the effect of the NABT on apoptosis would be the opposite of what is produced by the medical condition as reflected in Table 6A.

It follows from the AP Model that many “therapeutic solutions” exist for choosing the optimal NABT therapeutic (or combination thereof) to treat AP Diseases and Programming Disorders in accordance with the present invention. That is, several different NABTs—directed against different members of a select set of TR gene targets—may be active in treating the same disease. This situation is a direct consequence of the facts that

(a) the TRs involved in cellular programming are acting in an interdependent way as part of a combinatorial regulation system, and that

(b) different TR combinations can direct the same change in cellular programming.

The Reprogramming Test can be employed to optimize and characterize modifications to the NABTs for the treatment of an AP Disease or Programming Disorder. An exemplary test comprises the following:

(i) selecting the medical condition in question (Table 2) the subset of TRs and their direct modifiers, listed in Table 3 and/or the apoptosis modulators listed in Table 5, expressed by the AP Cells using both qualitative as well as quantitative measures, where the AP Cells come from patients with said medical condition as well as determining their expression by any appropriate cell lines or AP Cells from any appropriate animal models. Freshly obtained or recently explanted cells or tissues are most preferred for in vitro analysis;

(ii) comparing the effects of the modified NABT to unmodified NABT indexed in Table 7 (Sequences provided in Table 8 and which in the case of NABTs that are RNAi will be modified as described elsewhere herein) on expression levels of the target TRs and their direct modifiers and/or the apoptosis modulators selected in step (i) and also assessing expression levels in normal cells corresponding to the AP Cells, and/or in normal constitutively self-renewing normal tissue including but not limited to hematopoietic and gastrointestinal or, alternatively, making a similar determination for any other normal tissue that is to be therapeutically manipulated in accordance with this invention;

(iii) selecting one or more modified NABTs which show efficacious suppression of target gene expression in AP Cells from the relevant patients;

(iv) treating AP Cells and selected normal cells with NABTs prepared in step (iv) and scoring the effect on target gene expression and on cellular programming; and

(vi) selecting modified NABTs with desirable properties with respect to the therapeutic goal.

In a variation of the Reprogramming Test, the test is applied to determining which targets (found in Tables 3 and 5 and shown to be expressed by the cells of interest) and which NABTs (based on oligo sequences in Table 8) are suitable for the therapeutic reprogramming of normal cells including but not limited to normal stem cells as described elsewhere herein. In this embodiment, the AP Cells in the steps just outlined will be replaced by the normal cells of interest. Obviously, in this instance the requirement (found in the application of the Reprogramming Test to AP Diseases and Programming Disorders) that the normal cells of interest have a different TR or their direct modifier profile from the corresponding normal cells is not applicable.

Pathologic expression of an apoptosis program characterizes certain medical conditions that are not AP Diseases or Programming Disorders, (e.g., when expression of an apoptosis program is induced by an exogenous injury). Several of these are provided in Table 4B. The therapeutic goal in these conditions is to use an NABT to block apoptosis in the normal cells that may be affected via proximity to the injured tissue for example (Table 6B), without inducing concomitant undesirable effects on unaffected normal cells. NABTs suitable for treating these conditions can be assessed using the following steps:

(i) determining for the medical condition in question (Table 4B) the subset of the apoptosis modulators listed in Table 5, expressed by the affected cells using both qualitative as well as quantitative measures, where the affected cells preferably come from patients with said medical condition as well as determining their expression by similarly affected cell lines or by cells from animal models. Freshly obtained or recently explanted cells or tissues are most preferred for in vitro analysis;

(ii) determining which of apoptosis modulators detected in step (i) are also expressed by the corresponding unaffected normal tissue, or in unaffected normal constitutively self-renewing normal tissue including but not limited to hematopoietic and gastrointestinal;

(iii) selecting one or more gene targets for inhibition by NABTs and optionally, modified NABTs, on the basis of it being expressed by affected cells from the relevant patients;

(iv) preparing appropriate NABTs for the inhibition of said targets using the prototype sequences indexed in Table 7 and provided in Table 8 and which in the case of NABTs that are RNAi will be modified as described elsewhere herein;

(v) treating the affected cells and selected unaffected normal cells with NABTs prepared in step (iv) and scoring the effect on target gene expression and on cellular programming; and

(vi) selecting NABTs with desirable properties with respect to the therapeutic goal and further testing variants of these NABTs at step (v) where the variations include small changes in size and hotspot positioning as provided for by Table 8.

In yet another embodiment, the gene targets selected for inhibition are Molecular Risk Factors for particular medical conditions as shown in Table 11. The sequences for the prototype NABTs and size variants are provided in Table 8 and are indexed in Table 7.

The direct cause-and-effect associations identified by conventional approaches implicate certain Molecular Risk Factor target genes for therapeutic NABT inhibition. Some examples are the following with more examples provided in Tables 5, 6 and 11:
(1) β-amyloid precursor protein and apolipoprotein E 4 are causally implicated in the pathogenesis of Alzheimer's Disease;
(2) vascular endothelial growth factor (VEGF) is causally implicated in cancer, macular degeneration and in rheumatoid arthritis;
(3) TNF-alpha is causally involved in pathologic inflammatory conditions such as Arthritis, Crohn's Disease, psoriasis, and ankylosing spondylitis;
(4) TGF-beta is causally involved in fibrosis and Alzheimer's;
(5) PDGFR is causally involved in cancer and Alzheimer's;
(6) SGP2, or TRPM-2 is causally involved in cancer and Alzheimer's;
(7) ERK family members are causally involved in cancer and Alzheimer's;
(8) COX2 (prostaglandin endoperoxide synthase 2) is causally involved in inflammatory conditions such as arthritis as well as cancer and Alzheimer's, and;
(9) bax-alpha, bcl-2 alpha, bcl-2 beta, bcl-x, bcl-xl, fas/apo-1, ICE, ICH-1L and MCL-1 are molecules known to be causally involved in the regulation of apoptosis and, therefore, can be blocked by NABTs for the purposes of promoting or inhibiting apoptosis depending on the therapeutic needs of the situation.

In another embodiment, the present invention involves treating a medical condition with a NABT targeted to TRs or their direct modifiers that are known to regulate the expression of Molecular Risk Factor(s) for said medical condition. Note that the TR Ap-1 is a dimer made up of one jun family member (c-jun, junD, junB) and one fos family member (c-fos, fra-1, fra-2).

Certain medical conditions, Molecular Risk Factors and TRs as well as their direct modifiers are provided in Table 12 (the corresponding oligo or guide stand sequences for the NABTs listed are provided in Table 8). Some examples are the following: β-amyloid precursor protein and telomerase\human telomerase reverse transcriptase (hTERT) which are implicated in the production of certain disease processes including Alzheimer's and cancer respectively where, for example, the TRs SP1, SP3, SP4, Ap-1 (dimers consisting of jun and fos family members), AP-2, Ap-4, CREB, YY-1, Oct-1, Ets-2 and p53 are among those known to be involved in Alzheimer's and to regulate β-amyloid precursor protein expression; and MAD-1, Ets-2, c-myc, SP1, AP-1 and E2F-1 are involved in the control of telomerase\hTERT expression. Hence, blocking the expression and/or function of TR required for the expression of these medically important molecules will be therapeutically beneficial.

Genes encoded by the host cell are known to be important for the expression and functioning of infecting viruses. Indeed, blocking the action of NF-kappaB in HIV-infected cells by oligos has been shown to reduce HIV expression. Examples of virally-induced diseases that would benefit from such treatment include, but are not to be limited to, those caused by HIV, HTLV, CMV, herpes viruses, measles viruses, the hepatitis viruses, rhinoviruses, influenza viruses and hemorrhagic fever viruses. Host-encoded genes including, but not limited to TRs as well as their direct modifiers, that are known to regulate the pathogenic viruses and/or to affect pathologic effects on host cells are presented in Table 13 and include the following examples:
HIV: USF, Elf-1, Ap-1, Ap-2, Ap-4, Sp-1, Sp-3, Sp-4, p53, NF-kappaB, rel, GATA-3, UBP-1, EBP-P, ISGF3, Oct-1, Oct-2, Ets-1, NF-ATC, IRF-1, CDK-1, CDK-2, CDK-3, CDK-4, WAF-1, CDK-4;
CMV: SRF, NF-kappaB, p53, Ap-1, IE-2, C/EBP, Oct-1, Rb, CDK-1, CDK-2, CDK-3, CDK-4, WAF-1;
Herpesviruses: USF, Spi-1, Spi-B, ATF, CREB and C/EBP families, E2F-1, YY-1, Oct-1, Ap-1, Ap-2, c-myb, NF-kappaB, CDK-1, CDK-2, CDK-3, CDK-4, Cyclin B, WAF-1;
Hepatitis viruses: NF-1, Ap-1, Sp-1, RFX-1, RFX-2, RFX-3, NF-kappaB, Ap-2, C/EBP, Oct-1, Ets-2, CDK-1, CDK-2, CDK-3, CDK-4, WAF-1, Rb, E2F-1;
Influenza viruses: NF-kappaB, p53, YY-1, Ap-1, Oct-1, C/EBP, CDK-1, CDK-2, CDK-3, CDK-4, ERK, ERK-3, WAF-1; and
Papillomaviruses: CDK-1, CDK-2, CDK-3, CDK-4, WAF-1, ERK, ERK-3

Guidance relating to the administration or lack of administration of certain drugs with NABTs provided herein. For example, acetaminophen (paracetamol) and/or high dose antioxidants are precluded from use with the NABTs disclosed herein under certain circumstances. A metabolic product of acetaminophen, (NAPQI), binds to endogenous DNA when given to patients or animals and it also binds to bases in NABTs and thus affects their pharmacokinetics and therapeutic efficacy (See U.S. patent application Ser. No. 12/124,943; Rogers et al., Chem. Res. Toxicol. 10: 470, 1997). NAPQI is produced by cytochrome P450 and is highly reactive and, therefore, is short lived and does not leave the cells where it is produced. Accordingly, acetaminophen should not be given to patients receiving an NABT to inhibit gene expression in cells that express those cytochrome P450 isozymes known to produce NAPQI and other reactive metabolites of acetaminophen. Such cells include but are not limited to normal or diseased liver, kidney, lung, gastrointestinal tract, blood and endothelial cells as well as cancer cells. Cytochrome P450 isoenzymes and their pattern of tissue expression is more fully considered in the following: (1) Cytochrome P450: Structure, Mechanism and Biochemistry, PR Ortiz de Montellano, editor, 3^{rd} edition 2004, Springer, New York, N.Y.; and (2) Cytochrome P450: Role in the Metabolism and Toxicity of Drugs and other Xenobiotics, C Ioannides, editor, 1^{st} edition 2008, Royal Society of Chemistry, Cambridge UK.

Further, high dose antioxidants are known to induce cell cycle arrest, for example, by inducing p21 (12/124,943; Hsu et al., Anticancer Res. 25: 63, 2005; Weng et al., Biochem Pharmacol 69: 1815, 2005). Thus, high dose antioxidants (considered to be a daily dose of >500 on the USDA Oxygen Radical Absorbance Capacity Scale; Cao and Prior, Clin Chem 44: 1309, 1998) should not be given in combination with NABTs where the mechanism of action of the NABT requires the cells being targeted to traverse the cell cycle. This is particularly important, for example, for the treatment of cancer where NABTs used alone or in combination with genome damaging agents, such as many chemotherapeutic agents or ionizing radiation, are used to trigger the death of cancer cells as a result of DNA replication by said cancer cells. The targets for such NABTs for inhibition of expression would include but not be limited to the following genes and their RNA transcripts where each is known to promote cell cycle arrest in cells in response to chemotherapy or radiation: p53, Waf-1, Gadd 45, chk1 and chk2.

The following references provide more detail on which cancer chemotherapeutics bind to and/or otherwise damage endogenous DNA and, therefore, also damage NABTs. In a separate embodiment the use of the NABTs provided herein for the treatment of cancer in combination with such agents will administered according to dosage regimens that will allow the NABT time to fulfill its therapeutic purpose by avoiding the administration of such DNA damaging agents during this timeframe which is determined by the passage of at least one half-life of the DNA damaging agent(s). These references are incorporated herein by reference: (1) Physicians' Desk Reference (2008) 62nd edition, Thompson Heathcare Brooklyn, N.Y.; (2) Cancer: Principles & Practice of Oncology (2008) 8th edition VT DeVita et al., editors, Lippincott, Williams and Wilkins Philadelphia Pa.; (3) Cancer Medicine (2006) 7th edition DW Kufe editor, BC Decker Inc. Hamilton, Ontario Canada; (4) Cancer Chemotherapy & Biotherapy (2005) 4th edition BA Chabner and DL Longo editors, Lippincott, Williams and Wilkins Philadelphia Pa.; and (5) Goodman & Gilman's The Pharmacological Basis of Therapeutics (2005) 11th edition L Brunton, J Lazo and K Parker, McGraw-Hill New York, N.Y.

In other embodiments, drugs that affect TR expression and/or function are administered in approximate combination with (e.g., within the time frame of biologic activity) NABTs which modulate cellular programming. Such combinations can act synergistically to treat the disorder in question. Moreover, use in combination often allows use of lower doses than when treating the condition with a single agent. Of course the foregoing assumes such combinatorial approaches in no way inhibit the cellular reprogramming effect of the particular NABT(s).

Accordingly, other relevant modulators of TR expression and/or function used in conjunction with NABTs have utility for purposes that include but are not limited to the following: (1) To alter cellular programming in medical conditions where certain other drug or NABT modulators of TR expression and/or function are apt to be used in approximate combination with said NABT; and (2) where there is a rationale for using said NABT together with certain other modulators of TR expression and/or function to more effectively achieve a given therapeutic or other commercial purpose than could be achieved by the use of either agent alone. In the instance where said modulator of TR expression and/or function adversely affects said intended therapeutic purpose of a given NABT, then the use of said modulators of TR expression and/or function is contraindicated for use in combination with the NABT. In the instance where said modulator of TR expression and/or function promotes the intended therapeutic purpose of a NABT or establishes a new therapeutic or other commercial use, then the use of said modulators of TR expression and/or function in combination with NABT is indicated.

For example, NF-kappaB modulators are an important group of drugs that affect TR expression and/or function. NF-kappaB is a TR that plays a role in the regulation of cellular programming but is also active in inflammatory pulmonary, autoimmune, neurodegenerative and cardiovascular diseases as well as in cancer and osteoporosis. The following documents provide numerous examples of such NF-kappaB modulators that are either approved drugs or that are potential drugs in development along with, in many instances, their intended medical uses: Ahn et al., Current Mol Med 7: 619, 2007; Calzado et al., Current Med Chem 14: 367, 2007; O'Sullivan et al., Expert Opin Ther Targets 11: 111, 2007; Abu-Amer et al., Autoimmunity 41: 204, 2008; Uwe, Biochem Pharm 75: 1567, 2008; Guzman et al., Blood 110, 4427, 2007. A sampling of NF-kappaB drug activators includes, but is not limited to, the following: nicotine, anthracyclines (such as idarubicin), cyclohexamide, vinblastine and histone deacetylase inhibitors. A sampling of NF-kappaB drug and nutraceutical inhibitors includes but is not limited to the following: ibuprofen, salicylates, acetaminophen, flurbiprofen, sulindac, high dose antioxidants, IKK inhibitors, protease/proteasome inhibitors, certain anticancer protein kinase inhibitors including but not limited to flt-3 inhibitors, macrolide antibiotics, pentoxifylline, lisophylline, omega 3 fatty acids, rifampicin, statins, erythromycin, clarithromycin, artemisinin, (GSK)-3-beta inhibitor 4-benzyl-2-methyl-1,2,4-thiadiazolidine-3,5-dione (TDZD-8), parthenolide, parthenolide analogs including but not limited to dimethylaminoparthenolide, thalidomide and rolipram.

For some of these agents, NF-kappa B modulator activity was discovered fortuitously. An example of an approved drug that was developed for other reasons and then found to suppress NF-kappaB is choline magnesium trisalicylate. Cancer patients treated with this drug have been shown to have significantly reduced amounts of NF-kappaB in their cancer cells (Strair et al., Clin Cancer Res 14: 7564, 2008). In this and numerous other studies, NF-kappaB reduction by a variety of agents is associated with an increased sensitivity of cancer cells to conventional anticancer agents. Accordingly, such NF-kappaB inhibitors can be used beneficially in combination with those NABTs of the present invention that sensitize cancer cells to chemotherapy and/or radiation as well as to other agents capable of causing oxidative cellular damage or stress where said NABTs include but are not limited to those that inhibit p53, WAF-1, GADD-45, MCL-1, bcl-2 (alpha and beta), E2F-1, EGFR, BSAP, ID-1, junD, c-myc, Ets-1, Ets-2, KDR/FLK-1, NF-IL6, PDGFR, P1m-1, bcl-x, SGP2 (TRPM-2), TGF-beta, estrogen receptor, androgen receptor and VEGF. In addition the NF-kappaB inhibitors maybe NABTs of the present invention including but not limited to those targeting directly NF-kappaB and those indirectly targeting it for suppression including but not limited to those targeting Ref-1 or Id-1.

NABTs are commonly used as research reagents, including target validation for drug development, and diagnostics. For example, antisense NABTs are often used by those of ordinary skill in the art to elucidate the function of particular genes including but not limited to elucidating what microRNAs are regulated by what TRs. NABTs are also used, for example, to distinguish between functions of various members of a biological pathway. Antisense inhibition of gene expression has, therefore, been harnessed for research and drug development use.

Thus, another embodiment of the present invention involves diagnostic methods, NABT chemical and structural variants, and kits comprising the NABTs that are based on the sequences provided in Table 8. Expression patterns within cells or tissues treated with one or more NABT(s) can be compared to control cells or tissues not treated with NABTs and the patterns produced can be analyzed for differential levels of gene expression as they pertain, for example, to disease association, signaling pathways, cellular localizations, expression levels, cell size, cellular morphology, structures or functions of the genes examined. These analyses can be performed on stimulated or unstimulated cells and in the presence or absence of other compounds that affect expression patterns.

A novel semi-empirical method was developed by the present inventor for selecting the “hotspots” in the gene sequences used in the present invention as well as for selecting the prototype NABT antisense or guide stand sequences based on these hotspots. See Table 8 and guidance provided herein for guide and passenger strands of siRNA or dicer substrates. The most preferred size variants for NABTs are as follows: (1) conventional antisense with a RNase H mechanism of action (20 mers (range 14-34)); (2) conventional antisense with a steric hindrance mechanism with or without added RNase H mechanism of action (22 mers (range 14-34)); (3) siRNA (16 mers (range 14-25)); (4) dicer substrates (25-30 mers); and (5) expression vectors—at least the full length of the corresponding hot spot where the transcript containing said hot spot sequences and generated by the expression vector binds to untranslated exon sequences, a translational start site and/or splice junction in the target gene transcript. Thus, the prototype sequences provided for the latter types of NABTs (siRNA and dicer substrates) will preferably be size adjusted as provided for herein. The prototype sequences set forth in Table 8 were chosen as optimal for conventional antisense with backbone chemistries providing for target binding Tm values at physiologic salt near what is seen for phosphodiesters.

This semi-empirical method involves plugging in parameters chosen by the present inventor into the “Oligo” program (Version 3.4) created by Dr. Wojciech Rychlik (Rychlik and Rhoads, Nucleic Acids Res. 17: 8543, 1989; copyrighted 1989). These were initially arrived at intuitively and then tested in the lab and modifications made as necessary and the process repeated. This process was repeated until a final set of computer program parameters were identified. This method was then applied to more than 200 different gene sequences to determine the hotspots present in each target gene for which the NABTs of the invention were designed. Preliminary prototype sequences for each hotspot were then subjected to further culling on the basis of criteria chosen by the present inventor. The results are shown in Table 8. Hotspots define the antisense strand (called a guide strand in the case of RNAi) sequences which hybridize to the NABT causing an inhibition of the expression of the targeted gene.

Reports describing an early version of the AP Model involved the use of conventional antisense oligos to p53. Bayever et al. (Leuk Lymph 12: 223, 1994) have shown, for example, that such NABTs (SEQ ID NOS: 1-4) can be used to inactivate malignant stem cells from patients with acute myelogenous leukemia while not adversely affecting normal hematopoietic stem cells or more mature cells. The specific NABTs used in this study were phosphorothioates without additional modifications. SEQ ID NO: 4 is the subject of numerous other publications that show its anticancer and normal cell sparing effects.

| | | |
|---|---|---|
| SEQ ID NO. 1: | | 5′-AGTCTTGAGC ACATGGGAGG-3′ |
| SEQ ID NO. 2: | | 5′-ATCTGACTGC GGCTCCTCCA-3′ |
| SEQ ID NO. 3: | | 5′-GACAGCATCA AATCATCCAT-3′ |
| SEQ ID NO. 4: | OL(1)p53 | 5′-CCCTGCTCCC CCCTGGCTCC-3′ |

In addition to phosphorothioate these sequences (SEQ ID 1-4) have also been previously associated with dithioate, methylphosphonate or ethylphosphonate linkages (U.S. Pat. No. 5,654,415 and WO 93/03770).

These oligos (with SEQ ID NOS: 1-4 comprising the linkages just mentioned) have now been found to target four different “hot spot” regions of the p53 gene transcript which are suitable for attack by multiple different NABTs (e.g., p53 hot spots 14-17 in Table 8). The prototype and size variant sequences in Table 8 that are associated with these hot spots are surprisingly more effective in suppressing p53 expression than the original conventional antisense oligos (described in U.S. Pat. No. 5,654,415 and WO 93/03770) when the backbone chemistry is altered as described below.

For p53 hot spots 14 (SEQ ID NO: 3786) and 17 (SEQ ID NO: 3797) the most preferred prototype (SEQ ID NOS: 3787-3789 and SEQ ID NOS: 4 and 3789 respectively) and size variant oligo sequences listed in Table 8 are 2′-fluoro gapmers with phosphorothioate linkages, with FANA or LNA gapmers being preferred. More details concerning such gapmer oligos are provided elsewhere herein.

p53 hot spot 15 includes the primary translational start site for p53 while hot spot 16 includes the secondary translational start site. The present inventor has discovered that the use of certain conventional antisense oligos with a steric hindrance mechanism of action and directed to hot spot 15 or, alternatively combined use such an oligo with an oligo directed to hot spot 16 (Table 23) provides unexpectedly superior inhibitory properties when compared the original oligos having sequences provided in SEQ ID NOS: 2 and 3 with respect to the following: (1) their ability to suppress the expression of the p53 protein; and (2) demonstrating greater efficacy for use in the medical and other commercial applications listed in Table 11. The most preferred oligos for this purpose have 2′-fluoro substituted sugar analogs for all the nucleotides coupled with phosphorothioate linkages. Preferred chemistries for this purpose include the following: (1) morpholino or piperazine sugar substitution in all nucleosides; (2) LNA sugar substitution in all nucleosides with phosphorothioate linkages; and (3) FANA sugar modification in all nucleosides. More details on steric hindrance oligos suitable for use in the present invention are provided elsewhere herein.

For p53 hot spot 15 (SEQ ID NO: 3790), the associated prototype (SEQ ID NOS: 3791-3793) and corresponding size variant oligo sequences provided in Table 8 can also be used in oligos with an RNase H mechanism of action with surprisingly improved results (compared to the original oligos based on SEQ ID NO: 2). In this embodiment, 2′-fluoro gapmers with phosphorothioate linkages are most preferred. Also preferred are FANA or LNA gapmers. Table 8 lists for each hot spot (presented as an antisense sequence) at least one prototype conventional antisense or prototype RNAi oligo sequence along with a listing of size variant oligo sequences that are suitable for use in NABTs described. Each listing provides the hot spot sequence with each position (numbered right to left) according to the sense reference sequence (numbered left to right) provided along with the size variant antisense oligo sequences. In all sequences, the left most nucleoside is at the 5′ end. The size variant oligo sequences are presented as a number on a line that begins with the position number of the first nucleoside where the number representing the oligo provides the length of the sequence. Thus, the exact sequence for each size variant for each hot spot can be unequivocally read from the corresponding hot spot sequence using the position of the first base and the length of the sequence as provided in the table. The two junD antisense NABTs, H(1)junD (SEQ ID NO. 5) and H(2)junD (SEQ ID NO. 6) and one CREBP-1 antisense NABT, 13L, were tested on cancer cells and shown to have selective toxic activity on cancer cells. The cells tested were (AML blasts freshly obtained from patients and the following cancer cell lines 8226/Dox6, 8226 sensitive and Du-145. 8226 cells are from a patient with multiple myeloma. The D6 version of this line has been selected for doxorubicin resistance in vitro. The DU-145 line is from a patent with prostate cancer. The normal cells tested were bone marrow as described in Bayever et al. Leuk Lymph 12: 223, 1994. In brief, normal human bone marrow cells were incubated with from 10 nM to 10 μM of the NABTs of interest for 7 days. Viable cell counts were performed every two days following NABT treatment and the cells were then plated in mixed colony assays to determine what effects (if any) the NABTs would have on the proliferation and differentiation of various types of hematopoietic colony forming units.

| | | |
|---|---|---|
| SEQ ID NO: 5: | H(1)junD | GTCGGCGTGG TGGTGA |
| SEQ ID NO: 6: | H(2)junD | GCTCGTCGGC GTGGTGGTGA |
| SEQ ID NO: 552 | I3L | GTCCTTGTAT TGCCTGGC |

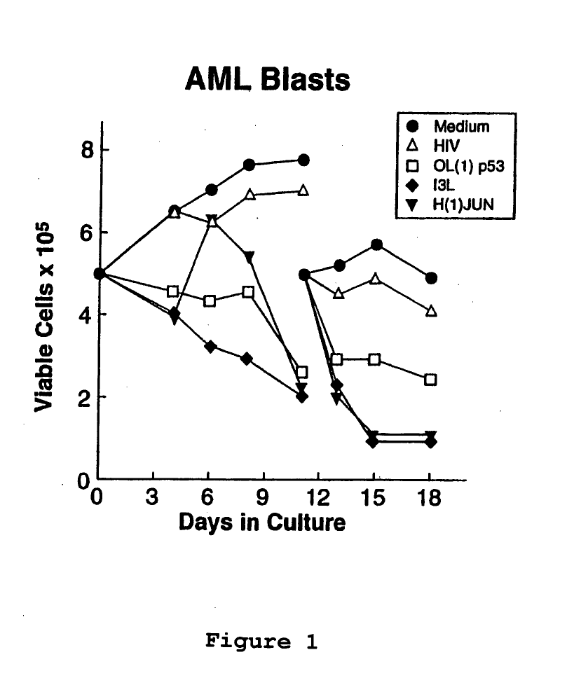

A representative example of the suspension culture data for 3 active NABTs is shown in FIG. 1 along with no NABT (medium) and a NABT control directed to an HIV sequence.

When the H(1)junD and H(2)junD NABTs were tested on malignant cell lines, they were found to have a diminished cytotoxic or anticancer growth-inhibitory effect than they had on freshly-obtained cancer cells. Surprisingly, these antisense NABTs could be used to dramatically sensitize various types of multidrug-resistant cancer cells to anti-cancer chemotherapeutic agents. Remarkably, these sensitizing effects were operative on cancer cells that have differing mechanisms for their multidrug resistance. Table 14 shows that H(1)junD or H(2)junD can be used to sensitize P-glycoprotein-expressing drug-resistant 8226/Dox6 cell line to vincristine, while H(1)junD also can sensitize DU-145 prostate cancer cells that express MRP and not P-glycoprotein (Table 14). These findings support the conclusion that suppressing the expression of junD, such as by treatment with antisense NABTs, can be used to reverse multidrug resistance resulting from multiple mechanisms. In contrast to the effects on multidrug resistant cancer cell lines, the H(1)junD NABT had minimal sensitizing potential when used to treat the drug-sensitive (parent) 8226 cancer cell line.

Antisense NABT represent a preferred embodiment of the invention. Antisense NABTs include the following: (1) conventional antisense oligos; (2) RNAi including (a) dicer substrates, (b) double stranded siRNA (siRNA) and (c) single stranded siRNA (ss-siRNA); as well as (3) expression vectors. The form of the NABT to be employed will depend on many factors, including: (1) the requirements of the relevant medical condition or commercial use; (2) the relative quality and nature of the various targeting sites for the gene of interest for NABT inhibition; (3) the cell type(s) expressing the gene to be inhibited; (4) the subcellular location(s) in which the relevant NABT concentrates; and (5) the desired duration or the NABT effect. For each parameter, there typically will be a multiplicity of effective NABT compositions that are suitable. Sequences having antisense properties for the three types of NABT listed above are provided in Table 8. When the NABT function as dicer substrates and siRNA, additional information is provided herein addressing modifications for ensuring that the sequences provided in Table 8 will be loaded into RISC as the guide (antisense) stand. Typically there are subtle differences between conventional antisense oligos and the antisense oligos that function in RNAi as guide strands, nevertheless some antisense oligos will have the capacity to function both as a conventional antisense oligo and as an RNAi guide strand.

Depending on factors considered herein, NABTs may be administered to patients and/or introduced into cells with or without a carrier. NABTs may be administered with or without being conjugated to a moiety that improves one or more of the ADME (absorption, distribution, metabolism and excretion) pharmacological characteristics of the NABT or administered in combination with an agent that improves one or more such ADME parameters. For many in vivo uses, conventional antisense NABTs or ss-siRNAs will be administered without a carrier. In contrast, for most in vivo and for in vitro uses NABTs that are double stranded siRNA or expression vectors will require a carrier. A given carrier may facilitate uptake of the NABT into many cell types or it may be designed such that uptake is cell-type specific. This flexibility allows for a substantial degree of control over which cell types will be subjected to the effects of any given NABT. This could allow, for example, for a given gene to be therapeutically inhibited in one tissue type while not being inhibited in another cell type where such an inhibition would otherwise cause an adverse effect.

The first conventional antisense oligos to be used clinically contained phosphorothioate backbones without additional modifications. Phosphothioates differ from normal DNA in that they have a sulfur replacing one of the non-bridging oxygens in the phosphodiester linkage. Such phosphorothioates will support RNase H cleavage of their target RNA but this backbone chemistry produces an antisense oligo with a lower binding affinity for its target than normal DNA. As a result, phosphorothioates tend to be less suitable for use in steric hindrance based inhibition methods than a number of other backbone chemistries. Use of phosphorothioate linkages is correlated with increased binding to plasma proteins, particularly albumin. In comparison to a number of other linkages that do not show a high level of binding to plasma proteins, phosphorothioates have prolonged plasma residence times and this in turn promotes tissue uptake.

Characteristics of phosphorothioates, related use and synthesis methods include, but are not limited to, those provided in the following U.S. Pat. Nos., 5,264,423, 5,276,019, 5,286,717, 5,852,168, 7,098,325, 6,399,831, 5,292,875, 5,003,097, 4,415,732; Zon and Geiser, Anticancer Drug Des 6: 539, 1991. The efficiency of phosphorothioate antisense NABTs can be further improved by the use of synthesis methods that produce oligos with diastereomerically enriched linkages that include, but are not limited to, those described in U.S. Pat. Nos. 5,734,041, 6,596,857, 5,945,521, 6,031,092, and 6,861,518 or where the 5′ and 3′ terminal end internucleoside linkages are chirally Sp and the internal internucleoside linkages are chirally Rp (U.S. Pat. No. 6,867,294).

The biological activities, particularly for in vivo use, of phosphorothioates as well as the other oligo backbone chemistries (such as but not limited to those with a 2′-fluoro group in at least some sugars or containing at least some FANA or LNA modified sugars and phosphorothioate linkages between at least some nucleosides as described) provided herein may also be improved in tissues and cell types with low oligo uptake by: (1) adding a 500-10,000 MW polyethyleneglycol (PEG) group to the 3′-end and a tocopheryl group to the 5′-end with the lower molecular weight PEG being preferred; or (2) adding a polymer to linked to an oligo at the 3′-end and/or at the 5′-end where the polymer is polyethyleneglycol and/or polyalkylene oxide and further where at least one such polymer has an average molecular weight of 0.05 kg/mol to about 50 kg/mol and where the polymers can be branched or linear. Alternatively, PEG can be replaced by a N-(2-hydroxypropyl)methacrylamide polymer. Characteristics, uses, methods and production of such oligos include but are not limited to those described in Bonora et al., Bioconjugate Chem 8: 793, 1997; Fiedler et al., Langenbeck's Arch Surg 383: 269, 1998; Vorobjev et al., Nucleosides & Nucleotides 18: 2745, 1999; US2005/0019761, WO 2008/077956, WO 01/32623.

Further modifications to phosphorothioates can provide additional attributes that confer advantages for certain uses. These include certain modifications of the sugars or their replacement by a piperazine ring thereby increasing the binding affinity for the target and in some instances also increasing stability in biological fluids. Modifications for this purpose include the following: (1) locked nucleic acids (LNA) with the alpha-L-LNA being preferred; (2) 2′-fluoro-D-arabinonucleic acids (FANA) with the S-2′F-ANA form being preferred as well as those with a piperazine ring replacing the nucleoside sugar moiety. Most preferred for the present invention is a backbone containing phosphorothioate linkages and ribose sugars modified by replacing the 2′ hydroxyl group with a fluorine moiety where the fluorine (2′ fluoro) is in the normal hydroxyl orientation in contrast to the fluorine orientation in FANA oligos. It is to be understood that the nucleoside or nucleotide monomers of RNA analogs, such as 2′ fluoro correspond to thymine (T) found in DNA may be replaced by the uracil (U) found in RNA. In addition, chimeric 2′-fluoro/2′-O-methoxyethoxy or 2′-O-methoxyethyl oligos are suitable for the practice of the current invention. Such antisense oligos have exceptionally high Tm values.

In addition to phosphorothioate linkages, other linkages suitable for use in the present invention include, but are not limited to, boranophosphate, phosphoramidate, phosphorodiamidate and phosphorodiamidate with side groups attached to at least some linkages where the side group supplies a positive charge. Boranophosphate linkages can be used with deoxyribose sugars or certain deoxyribose analogs to form backbones that will support RNase H activity. Phosphoramidate, phosphorodiamidate and phosphorodiamidate with side group supplying a positive charge are linkages that have the advantage of increasing the binding affinity of the oligo for its target sequence and are the most preferred linkages for use in conventional antisense morpholino or piperazine oligos that have a steric hindrance mechanism of action.

Characteristics and synthesis of 2′ fluoro oligos including gapmers are described in, but not limited to, the following: Kawasaki et al., J Med Chem 36: 831, 1993; Cummins et al., Nucleic Acids Res 23: 2019, 1995; Sabahi et al., Nucleic Acids Res 29: 2163, 2001; Monia et al., J Biol Chem 268: 14514, 1993; Blidner et al., Chem Biol Drug Des 70: 113, 2007; Egli et al., Biochem 44: 9045, 2005; Schultz and Gryaznov, Bhat et al., Nucleic Acids Res 52: 69, 2008; WO93/13121, WO97/31009 and WO2007/090073.

LNA characteristics and synthesis methods include, but are not limited to, those provided in Braasch et al., Biochem 42: 7967, 2003; Jepsen and Wengel, Curr Opinion Drug Dis & Dev 7: 188, 2004; Grunweller et al., 31: 3185, 2003; Pfundheller et al., Methods Mol Biol 288:127, 2005; Gaubert and Wengel, Nucleosides Nucleotides Nucleic Acids 22: 1155, 2003; Wengel et al., Nucleosides Nucleotides Nucleic Acids 22: 601, 2003; Kumar et al., Bioorg Med Chem Lett 18: 2219, 1998; WO0125248, WO07107162, WO04106356, WO03095467, WO03039523, WO03020739, WO0066604, WO0056748, WO9914226, U.S. Pat. No. 7,084,125, U.S. Pat. No. 7,060,809, U.S. Pat. No. 7,053,207, U.S. Pat. No. 7,034,133, US20050287566, US20040014959, U.S. Pat. No. 6,794,499, US20030224377, US20030144231, US20030134808, US20030087230, US20030082807, U.S. Pat. No. 6,670,461, US20020068708, US20040038399, US20050233455, US20050142535. LNA oligos including gapmers and other variants are commercially available from Sigma-Genosys.

FANA oligo characteristics and synthesis methods include but are not limited to those provided in Ferrari et al., Ann NY Acad Sci 1082: 91, 2006; Wilds and Damha, Nucleic Acids Res 28: 3625, 2000; Lok et al., Biochem 41: 3457, 2002; Min et al., Bioorganic & Med Chem Lett 12: 2651, 2002; Kalota et al., Nucleic Acids Res 34: 451, 2006; US20040038399, US20050233455, US20050142535, WO06096963, WO03064441, WO0220773, WO03037909.

Characteristics and synthesis of oligos with a piperazine ring substitution for the normal ribose or deoxyribose sugar include, but are not limited to, those described in U.S. Pat. No. 6,841,675 and herein. Piperazine containing oligos (piperazines or piperazine oligos) with phosphodiester, linkages can be used as such or sulfurized to generate phosphorothioate linkages using the standard methods contained in the references and patents listed above. Other suitable linkages for the NABTs containing the piperazine ring in place of the normal furanose ring include, for example, boranophosphate, amide, phosphonamide, phosphorodiamidate; phosphorodiamidate with side group supplying a positive charge, carbonylamide, carbamate, peptide and sulfonamide. Such oligos, with at least one piperazine ring replacing a furanose ring in a nucleoside or nucleotide (preferably with at least four such replacements) and linked by at least one phosphorothioate or boranophosphate and preferably with at least 10 such linkages including those arranged as conventional gapmers are useful conventional antisense NABTs for the practice of the current invention.

Conventional antisense oligos solely made up of linked LNA, FANA or 2′-fluoro modified nucleoside often exhibit a reduced amount of RNase H activity against their target, if any. One established way to gain RNase H activity in such molecules is to produce gapmers in which the central nucleosides in the NABT have deoxyribose as the preferred sugar moiety, combined with a linkage such as boranophosphate or phosphorothioate that can support RNase H when used as part of a DNA analog. LNA, FANA or 2′ fluoro gapmer NABTs are 16-22mers with phosphorothioate or boranophosphate linkages and a 4-18 nucleoside core flanked by sequences that do not readily support RNase H activity (those containing LNA, FANA or 2′ fluoro containing nucleosides) and which flanking sequences are no more than two nucleosides different in length. The 4-18 nucleoside core uses normal deoxyribose or a suitable analog as the sugar that will support RNase H cleavage of the target RNA to which the oligo is hybridized. Phosphodiester linkages also may be used for in vitro applications where nuclease activity is reduced. Most preferred are 20-mer 2′ fluoro gapmers with an 8 nucleoside core and phosphorothioate linkages throughout as illustrated below. The x's represent different bases (A, G, U/T or C) that are part of a series of linked nucleosides while the capital x's represent nucleosides with 2′ fluoro modifications to the sugar and the small x's represent nucleosides with deoxyribose sugar. The ˜ symbol represents the phosphorothioate linkage. RNA analogs (e.g., 2′ fluoro oligos are typically but not necessarily produced using uracil rather than thymidine bases.

5′-X~X~X~X~X~X~x~x~x~x~x~x~x~x~X~X~X~X~X~X-3′

Variant gapmers with sugars containing 2′-O-methyl, 2′-O-ethyl, 2′O-methoxyethoxy or 2′-O-methoxyethyl groups in the flanking sequences can also be used but are less preferred than LNA, FANA or 2′ fluoro modifications with the 2′ fluoro modification being most preferred. In addition to the documents provided above, synthetic processes for generating oligos with variable combinations of nucleoside linkages including, but not limited to phosphodiester, phosphorothioate, phosphoramidate and boranophosphate including those for promoting RNase H activity against the RNA target are also presented in WO2004/044136, WO0047593, WO0066609, WO0123613, U.S. Pat. No. 6,207,819 and U.S. Pat. No. 6,462,184.

In another approach to improve the ability of conventional antisense oligo NABTs to promote RNase H activity against their target, nucleosides with certain base modifications can be inserted at a single position near the center (within 4 nucleosides of either the 5′ or 3′ end) of FANA, LNA, 2′ fluoro or piperazine oligos, as well as at the junction between a series of RNA or RNA-analog nucleoside and a series of DNA or DNA analog nucleoside or the reverse in FANA, LNA, 2′ fluoro, 2′-O-methyl, 2′-O-ethyl 2′-O-methoxyethoxy or 2′-O-methoxyethyl gapmer antisense oligos to achieve or further promote RNase H cleavage of the target RNA. The promotion of RNase H activity by this means appears to be due to added flexibility to the strand that is needed for promoting RNase H activity without interfering with the recognition of the NABT:RNA hybrid as a suitable substrate. The specific base modifications that can be used for this purpose and inserted either at gapmer junctions or near the center of the oligo are selected from the group consisting 4′-C-hydroxymethyl-DNA, 3′-C-hydroxymethyl-ANA, or piperazino-functionalized C3′,02′-linked-ANA where ANA refers to an arabinonucleic acid. Modified nucleotides or nucleotides that can be inserted at gapmer junctions for the purpose of promoting RNase H activity are selected from the group consisting of 2′ fluoro-arabinonucleotides, abasic, tetrahydrofuran (THF). For example, those with the bases shown in Formulas I, II and III, and those with bases selected from Formulas IV-XII or with the structures shown in Formulas XIII-XVII would be suitable for use in the present invention. Formula XVIII shows the structure of THF nucleotides and Formula XIX abasic nucleotides. The specific chemical structure of these base modified nucleosides and the synthesis of oligos containing them include, but are not limited to, those described in Vester et al., Bioorganic & Med Chem Lett 18: 2296, 2008 and US2008/0207541.

Formulas I-XIX are set forth below:

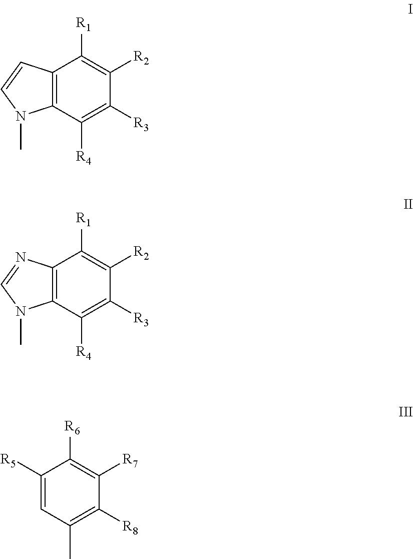

wherein each of _{R1-8} is independently selected from H, halogen and C₁₋₃ alkyl. R₈ may also be independently selected from fluorine and methyl. In certain embodiments, nucleobase is selected from Formulas IV, V, VI:

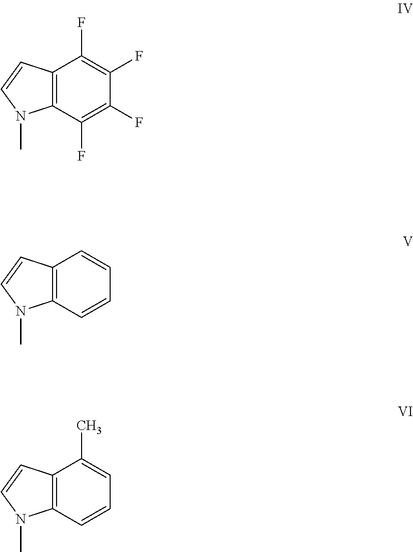

or Formulas VII, VIII, IX, X or XI

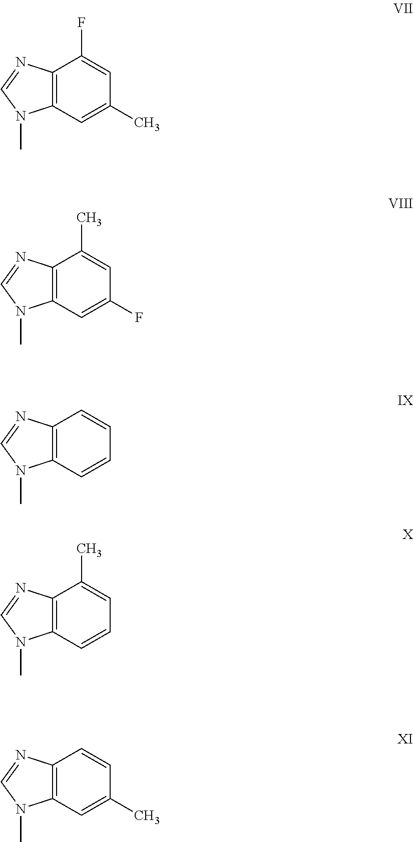

or formulas XII or XIII:

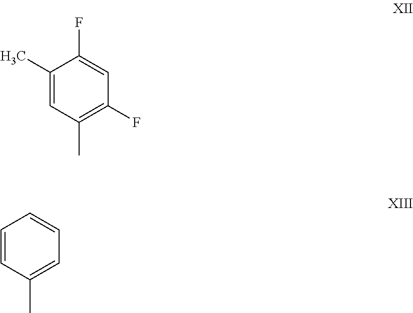

In some embodiments, the invention provides compounds of the Formula: (T₂)ⱼ-(T₃)ₖ-(T₁)ₘ-(T4)ₙ-(T₁)p-(T₅)_{q}-(T₂)ᵣ

wherein
each T₁ is a T-deoxyribonucleotide;
each T₂ is a nucleotide having a higher binding affinity for a RNA target as compared to the binding affinity of a 2′-deoxyribonucleotide for said RNA target;
each T₃, T₄ and T₅ are transition moietys;
j and r independently are 0 to 10, and together the sum of j and r is at least 2;
m and p independently are 1 to 20, and together the sum of m and p is at least 5;
k, n and q independently are 0 to 3, and together the sum of k, n and q is at least 1.
In some embodiments, T₂ comprises a nucleotide having a northern conformation.
In some such embodiments, T₂ comprises a nucleotide having a 2′-modification.
In some embodiments, j and r are each from 2 to 5, and m is 10 to 16. In some embodiments, j is 2, r is 2 and m is 14-18. In some embodiments, j is 2, r is 2 and m is 16. In some embodiments, j is 4, r is 4 and m is 10-14. In some embodiments, j is 4, r is 4 and m is 12. In some embodiments, j is 5, r is 5 and m is 8-12. In some embodiments, j is 5, r is 5 and m is 10.

In some embodiments, the invention provides methods of increasing one of the rate of cleavage or the position of cleavage of a target RNA by RNase H comprising:

selecting an oligonucleotide having an RNase H cleaving region and a non-RNase H cleaving region;
selecting a transition moiety capable of modulating transfer of the helical conformation characteristic of an oligonucleotide bound to its 3′ hydroxy to an oligonucleotide bound to its 5′ hydroxyl;
interspacing said transition moiety in said oligonucleotide positioned between said RNase H cleaving region and said non-RNase H cleaving region; and
binding said oligonucleotide to said target RNA in the presence of RNase H.

In certain embodiments, the oligonucleotide has the Formula: (T₂)ⱼ-(T₃)ₖ-(T₁)ₘ-(T₄)ₙ-(T₁)p-(T₅)_{q}-(T₂)ᵣ

In certain embodiments, the transition moiety bears a nucleobase having one of the structures IV-XIII, supra.

Structures of the modifications designed to introduce conformational flexibility (transition moieties) into the heteroduplex include: the propyl (C3), butyl (C4), pentyl (C5) hydrocarbon linkers; tetrahydrofuran (THF), abasic and ganciclovir modifications as well as 2-fluoro-6-methylbenzoimidazole, 4-methylbenzoimidazole, and 2,4-difluorotoluoyl deoxyribonucleotides. Gapmers designed to treat viral diseases responsive to gancyclovir such as those caused by CMV can find added benefit by employing the gancyclovir modification.

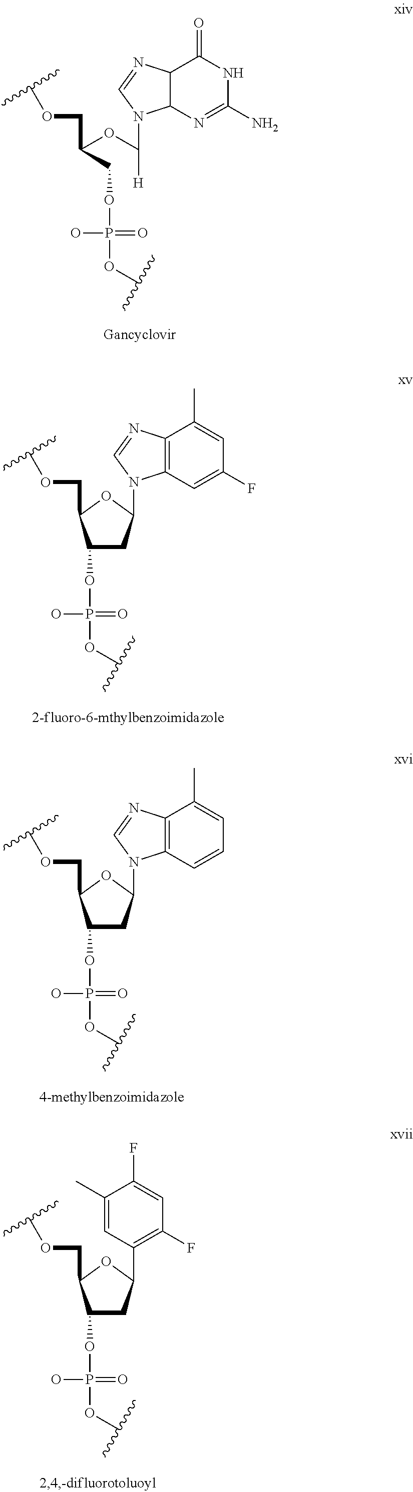

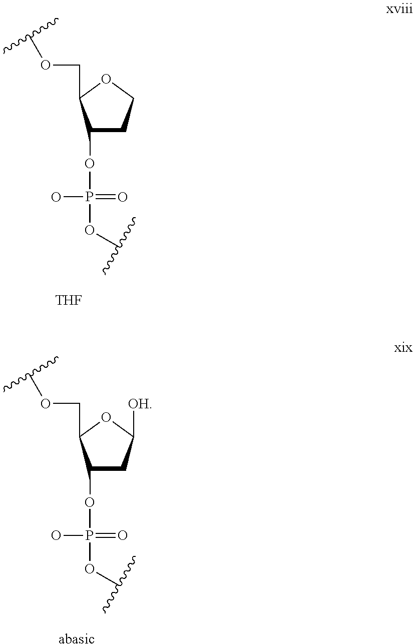

In yet another approach certain acyclic nucleoside or non-nucleotidic linkers can be inserted respectively in place of, or between, one or two nucleosides at or near the center of otherwise pure FANA, LNA, 2′ fluoro, morpholino, phosphorothioate, boranophosphate, 2′-O-methyl, 2′-O-ethyl, 2′-O-methoxyethoxy or 2′-O-methoxyethyl antisense oligos or their gapmers or into piperazine oligos to achieve or further promote the ability of the NABT to support RNase H cleavage of its target. These linkers also can be placed at the junctions between a series of RNA or RNA-analog nucleoside and a series of DNA or DNA analog nucleoside or the reverse in FANA, LNA, 2′ fluoro, 2′-O-methyl, 2′-O-ethyl 2′-O-methoxyethoxy or 2′-O-methoxyethyl gapmer antisense oligos. These linkers provide added flexibility to the strand needed for promoting RNase H activity without interfering with the recognition of the NABT:RNA hybrid as a suitable substrate. A preferred conventional antisense NABTs for this purpose has FANA modified oligonucleotides while 2′-fluoro oligos with the fluorine in the normal hydroxyl stereochemical configuration are most preferred and the linker to be used is a propyl (C3′), butyl (C4′), pentyl (C5′) or C₃-C₆ alkylene or single peptide bond preferably placed near the middle of the NABT or between one of the next three nucleosides closer to the 3′ end. The specific chemical structure of these linkers, their promotion of RNase H cleavage of the RNA targeted by antisense oligos containing them and the synthesis of such oligos include but are not limited to those described in Vorobjev et al., Antisense & Nucleic Acid Drug Dev 11: 77, 2001; Patureau et al., Bioconjugate Chem 18: 421, 2007; Mangos et al., J AM Chem Soc 125: 654, 2003; WO03037909, US2005/0233455, US2008/0207541.

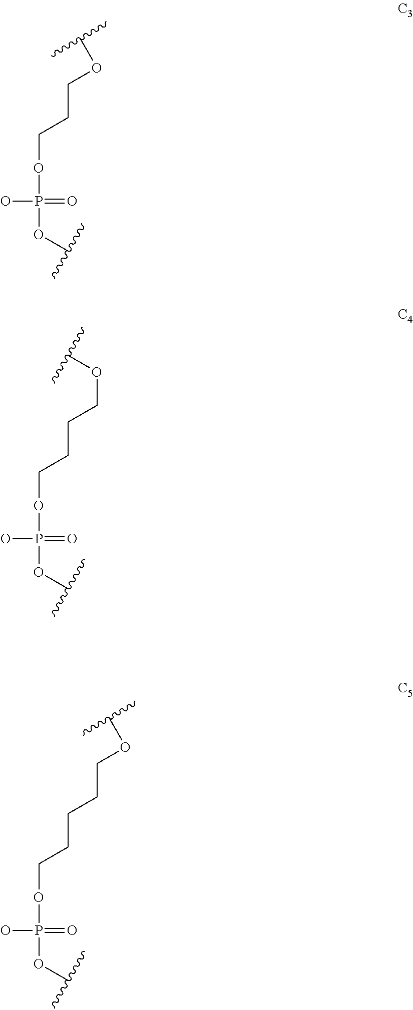

Published application US2008/0207541 includes the design considerations for using such linkers in hybrid oligos with different regions with two different conformations one of which is consistent with promoting RNase H activity (such as deoxynucleotides) against its target RNA and another region that is not (such as 2′-O-alkoxyalkyl ribonucleotides). The use of such linkers in this context preferably involves locating the linker between regions with conformational differences. In the case of piperazine oligos, these methods can be used to place an acyclic nucleotide, alkyl, oligomethylenediol or oligoethylene glycol linker in an otherwise phosphodiester or phosphorothioate linked oligo or a peptide linker in a peptide linked oligo.

Of these various methods for improving RNase H activity the most preferred for the present invention are modifications involving conventional antisense 2′ fluoro oligos including those with a gapmer design where the method involves the use of THF or abasic nucleosides or propyl or butyl linkers as described herein and the linkages between the nucleosides are phosphorothioate.

Boranophosphate linkages can be used in place of phosphorothioate linkages to stabilize conventional antisense NABTs with respect to nuclease attack while also providing for RNase H dependent cleavage of the target RNA in the context of a DNA analog (which in the case of a gapmer may be limited to the central portion of the backbone). The properties and synthesis of boranophosphates include but are not limited to those covered in the following: Li et al., Chem Rev 107: 4746, 2007; Summers and Shaw, Current Med Chem 8: 1147, 2001; Rait and Shaw, Antisense & Nucleic Acid Drug Dev 9: 53, 1999; Shimizu et al., Org Chem 71: 4262, 2006; Wada et al., Nucleic Acids Symp Series 44: 135, 2000; WO00/00499; U.S. Pat. No. 6,160,109, U.S. Pat. No. 5,130,302; U.S. Pat. No. 5,177,198; U.S. Pat. No. 5,455,233; U.S. Pat. No. 5,859,231).

A second mechanism whereby conventional antisense can inhibit the expression of a particular gene is through steric hindrance. RNA and DNA target sites suitable for conventional antisense oligo attack of this type include 1) primary and secondary translational start sites (oligos in Table 8 that contain a CAT, CAC, CAA, CAG, TAT, CGT or CAG motif where it is understood that T become U in the RNA transcript); 2) 5′-end untranslated sites involved in ribosomal assembly (sequences in Table 8 that occur upstream of the first CAT motif); and 3) sites involved in the splicing of pre-mRNA (SEQ IDS NOS: 2806-2815 in Table 8). A primary translational start site is the one most often used by a particular cell or tissue type. A secondary translational start site is one that is used less often by a particular cell or tissue type. The use of the latter may be determined by natural cellular processes or may be the result of inhibition of the use of the primary translational start site such as would occur when the said cells are treated with an NABT directed to the primary translational start site in question. Thus, the complete inhibition of the expression of a particular gene could require the use of two or more NABTs where one is directed to the primary translational start site and one or more additional NABTs are directed to secondary translational start sites.

NABT backbone configurations that demonstrate particularly high binding affinities to the target (measured by melting temperature or Tm) are preferred for implementing the steric hindrance mechanism. LNA, FANA, 2′-fluoro, morpholino and piperazine containing backbones are particularly well suited for this purpose. Most preferred are 22-mer 2′ fluoro oligos with phosphorothioate linkages throughout as illustrated below. The x's represent different bases (A, G, U/T or C) that are part of a series of linked nucleosides with 2′ fluoro modifications to the sugar. The ˜ symbol represents the phosphorothioate linkage. In RNA analogs 2′ fluoro oligos typically, but not necessarily, are produced with uracil rather than thymidine bases.

5′-X~X~X~X~X~X~X~X~X~X~X~X~X~X~X~X~X~X~X~X~X~X-3′

Phosphorothioate and boranophosphate linkages typically lead to a reduction in binding affinity with the target RNA but they may improve pharmacokinetics of an NABT by causing it to bind to plasma proteins. The potential pharmacokinetic advantages provided by these linkages, however, are not necessary in the case of backbones containing morpholino or piperazine substitutions for the sugar.

In the case of NABTs with other nucleoside chemistries and linkages than phosphorothioate, or boranophosphate, plasma protein binding, however, can be improved by covalently attaching to it, or to a carrier associated with it, a molecule that binds a plasma protein such as serum albumin. Such molecules include, but are not limited, to an arylpropionic acid, for example, ibuprofen, suprofen, ketoprofen, pranoprofen, tiaprofenic acid, naproxen, flurpibrofen and carprofen (U.S. Pat. No. 6,656,730).

Morpholino oligos are commercially available from Gene Tools LLC. Morpholino oligo characteristics and synthesis include but are not limited to those presented in the following: Summerton and Weller, Antisense Nucleic Acid Drug Dev 7: 187, 1997; Summerton, Biochim Biophys Acta 1489: 141, 1999; Iversen, Curr Opin Mol Ther 3: 235, 2001; U.S. Pat. No. 6,784,291, U.S. Pat. No. 5,185,444, U.S. Pat. No. 5,378,841, U.S. Pat. No. 5,405,938, U.S. Pat. No. 5,034,506, U.S. Pat. No. 5,142,047, U.S. Pat. No. 5,235,033. Morpholino oligos for the purposes of the present invention may have the uncharged and/or at least one cationic linkages between the nucleoside analogs made up of a morpholino ring and a normal base (guanine, uracil, thymine, cytosine or adenine) or a unnatural base as described herein. The preferred linkage for morpholino oligos is phosphorodiamidate which is an uncharged linkage. In some embodiments it may be modified as discussed below to provide a positive charge.

In one embodiment, the morpholino subunit has the following structure:

### Schematic of a Morpholino Subunit

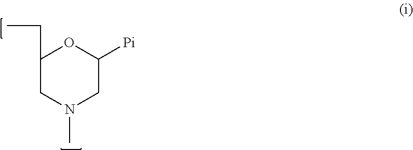

where Pi is a base-pairing moiety, and the linkages depicted above connect the nitrogen atom of (i) to the 5′ carbon of an adjacent subunit. The base-pairing moieties Pi may be the same or different, and are generally designed to provide a sequence which binds to a target nucleic acid.

The use of embodiments of linkage types (b1), (b2) and (b3) above to link morpholino subunits may be illustrated graphically as follows:

### Schematic of Linkages for Morpholio Subunit

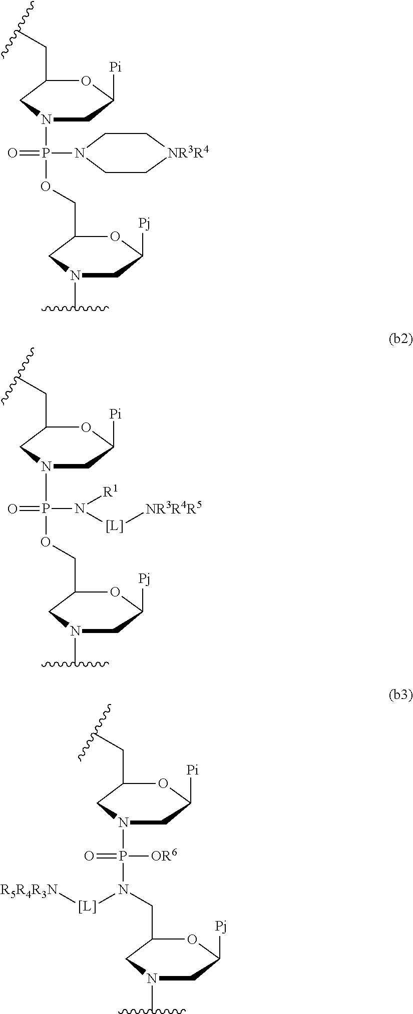

Preferably, at least 5% of the linkages in an oligo are selected from cationic linkages (b1), (b2), and (b3); in further embodiments, 10% to 35% of the linkages are selected from cationic linkages (b1), (b2), and (b3). As noted above, all of the cationic linkages in an oligo are preferably of the same type or structure.

In further embodiments, the cationic linkages are selected from linkages (b1′) and (b1″) as shown below, where (b1″) is referred to herein as a “Pip” linkage and (b1″) is referred to herein as a “GuX” linkage:

In the structures above, W is S or O, and is preferably O; each of R1 and R2 is independently selected from hydrogen and lower alkyl, and is preferably methyl; and A represents hydrogen or a non-interfering substituent on one or more carbon atoms in (b1′) and (b1″). Preferably, each A is hydrogen; that is, the nitrogen heterocycle is preferably unsubstituted. In further embodiments, at least 10% of the linkages are of type (b1′) or (b1″); for example, 20% to 80%, 20% to 50%, or 20% to 30% of the linkages may be of type (b1′) or (b1″). In other embodiments, the oligo contains no linkages of type (b1′). Alternatively, the oligo contains no linkages of type (b1) where each R is H, R³ is H or CH₃, and R⁴ is H, CH₃, or an electron pair.

In still further embodiments, the cationic linkages are of type (b2), where L is a linker up to 12 atoms in length having bonds selected from alkyl (e.g. —CH₂—CH₂—), alkoxy and alkylamino (e.g. —CH₂—NH—), with the proviso that the terminal atoms in L (e.g., those adjacent to carbonyl or nitrogen) are carbon atoms.

The morpholino subunits may also be linked by non-phosphorus-based intersubunit linkages, as described further below, where at least one linkage is modified with a pendant cationic group as described above. For example, a 5' nitrogen atom on a morpholino ring could be employed in a sulfamide linkage or a urea linkage (where phosphorus is replaced with carbon or sulfur, respectively) and modified in a manner analogous to the 5'-nitrogen atom in structure (b3) above.

The subject oligo may also be conjugated to a peptide transport moiety which is effective to enhance transport of the oligo into cells. The transport moiety is preferably attached to a terminus of the oligo.

### Schematic of Attachment of a Cell Penetrating Peptide to Morpholino Backbone

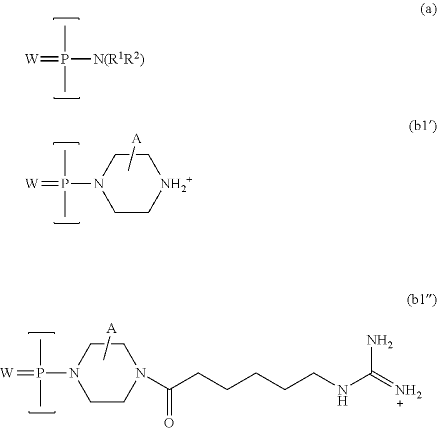

In the structures above, W is S or O, and is preferably O; each of R¹ and R² is independently selected from hydrogen and lower alkyl, and is preferably methyl; and A represents hydrogen or a non-interfering substituent on one or more carbon atoms in (b1′) and (b1″). Preferably, each A is hydrogen; that is, the nitrogen heterocycle is preferably unsubstituted. In further embodiments, at least 10% of the linkages are of type (b1′) or (b1″); for example, 20% to 80%, 20% to 50%, or 20% to 30% of the linkages may be of type (b1′) or (b1″). In other embodiments, the oligo contains no linkages of type (b1′). Alternatively, the oligo contains no linkages of type (b1) where each R is H, R³ is H or CH₃, and R⁴ is H, CH₃, or an electron pair.

In still further embodiments, the cationic linkages are of type (b2), where L is a linker up to 12 atoms in length having bonds selected from alkyl (e.g. —CH₂—CH₂—), alkoxy (—C—O—), and alkylamino (e.g. —CH₂—NH—), with the proviso that the terminal atoms in L (e.g., those adjacent to carbonyl or nitrogen) are carbon atoms.

The morpholino subunits may also be linked by non-phosphorus-based intersubunit linkages, as described further below, where at least one linkage is modified with a pendant cationic group as described above. For example, a 5' nitrogen atom on a morpholino ring could be employed in a sulfamide linkage or a urea linkage (where phosphorus is replaced with carbon or sulfur, respectively) and modified in a manner analogous to the 5'-nitrogen atom in structure (b3) above.

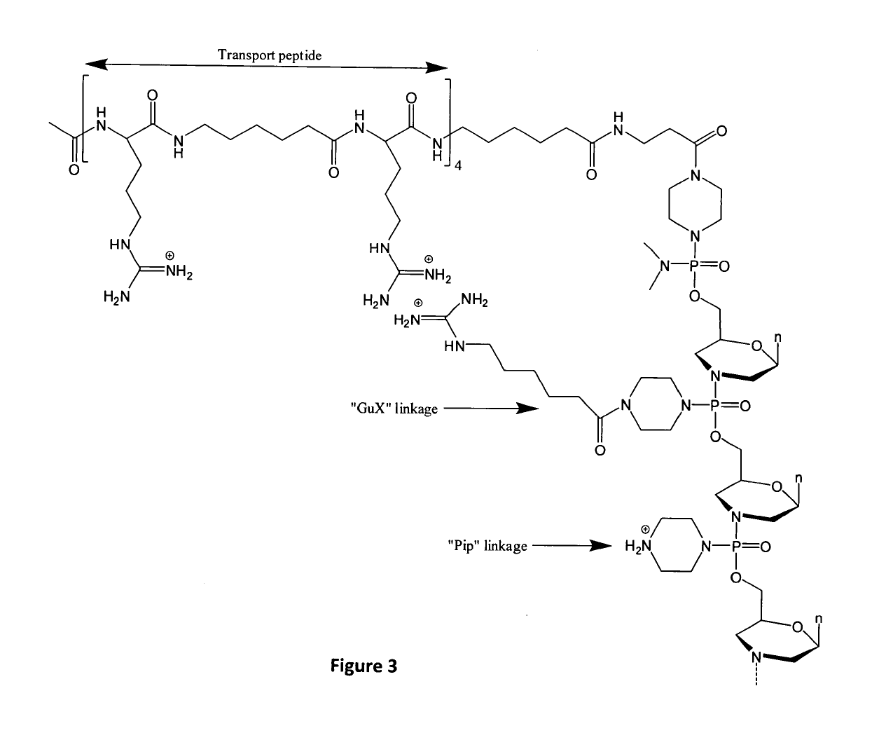

The subject oligo may also be conjugated to a peptide transport moiety which is effective to enhance transport of the oligo into cells. The transport moiety discussed further hereinbelow and is preferably attached to a terminus of the oligo, as shown, for example, in FIG. 3.

Also preferred are NABTs that comprise a piperazine ring in the place of the ring ribose or deoxyribose sugar. Such analogs are described in U.S. Pat. No. 6,841,675 to Schmidt et al. Methods for synthesizing piperazine based nucleic acid analogs are also disclosed in the '675 patent. Such substitutions improve in vivo bioavailability and exhibit lower aggregation characteristics. The amino acid-derived sidechain functionality denoted R² and R³ in the formula below is unique. This region of the molecule provides useful biological and medicinal applications beyond antisense nucleobase/nucleobase interactions and hydrogen bonding. In some embodiments of the instant invention, nucleoside analogs represented by the following formula are included:

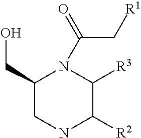

The formula shows the schematic representation of this embodiment with R¹ selected from the group consisting of adenine, thymine, uracil, guanine and cystosine. R² and R³ are side chain groups derived from amino acids and amino acid analogs, or any diastereoisomeric combinations thereof. As such, R² and R³ may be selected from the group consisting of hydrogen and/or all sidechains occurring in the 20 natural amino acids in all isomeric and diastereoisomeric forms and derivatives thereof, such as, but not limited to Serine=CH₂ OH, and Lys=(CH₂)₄ NH₂. In other embodiments, the nucleobase is a nucleobase derivative selected from the group consisting of inosine, fluorouracil, and allyluracil. The nucleobase may further be chosen from a group of nucleobase analogs including daunamycin, and other polycyclic or aromatic hydrocarbon residues known to bind to DNA/RNA.

In many of these embodiments, the piperazine nucleic acid analogs may be so configured as to be capable of forming a phosphoramidite, sulfonamide, phosphorodiamidate, phosphorodiamidate modified to have a positive charge as described for certain morpholino oligos or carbonylamide backbone linkage. They may also generally be rapidly assembled in a few synthetic steps from commercial grade materials. The length of the linkage between piperazine rings in the NABT of the instant invention may vary from one to four carbons in length, and may be branched or unbranched. The NABTs of the instant invention are also compatible with standard solid phase synthesizers, and may thus be used with synthesizers currently used in the art to allow easy assembly of molecules containing them.

The invention further comprises amide-, phosphonamide-, carbamate-, and sulphonamide-linked oligos made up of homo-oligonucleotides or comprising a chimera of either DNA or RNA and the nucleoside analogs of the instant invention. In some embodiments, the oligo is a composition containing a number, n, of nucleoside monomers represented by the formula:

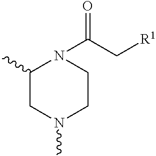

wherein R¹ is a nucleobase selected from the group consisting of adenine, thymine, uracil, guanine, and cytosine; wherein n is from about 1 to about 30; and wherein the nucleoside monomers are joined by amide-, phosphonamide-, carbamate-, or sulfonamide-linkages. In some of these embodiments, R¹ may be a nucleobase derivative selected from the group consisting of inosine, fluorouracil, and allyluracil. In others, the nucleobase derivative is chosen from a group including daunamycin and other polycyclic or aromatic hydrocarbon residues known to bind to DNA/RNA. In some of these oligonucleotide compositions n is from about 1 to about 30. The invention further includes oligos containing branching from the sidechains of the amino acids, rings of oligos and other tertiary, non-linear structures.

As previously noted, in some of these oligonucleotide compositions, phosphodiester linkages join the monomers. In some of these, the phosphodiester bonds comprise a linker of between about 1 and about 4 carbons in length. In others the monomers are joined by peptide bonds. In some of these, the peptide bonds comprise a linker of between about 1 and about 4 carbons in length. Finally, in other embodiments, sulfonamide bonds join the monomers. In some of these, the sulfonamide bonds comprise a linker of between about 1 and about 4 carbons in length. In other embodiments, carbamate linkages join the monomers. In some of these, the carbamate bonds consist of a linker of between 1 to 4 carbons in length. Included are also all possible chimeric linkages of the possible structures.

Since the steric hindrance mechanism is not dependent on RNase H activity, NABTs using this mechanism have the potential to be active in cells where RNase H levels are too low to adequately support conventional antisense oligo effects dependent on this mechanism. Stem cells an early progenitor cells have adequate levels of RNase H for this purpose while cells that have differentiated beyond the stem or progenitor cell stage typically do not. When functional, however, NABTs that support the RNase H based mechanism have the potential advantage over steric hindrance based mechanism of working catalytically since the same NABT molecule is capable of inactivating numerous target RNA molecules. As discussed elsewhere herein it is also possible to modify LNA, FANA, 2′-fluoro, morpholino and piperazine containing backbones to enable or increase their potential to catalyze the cleavage of their target RNA by RNase H by inserting certain linkers, acyclic nucleosides or by using the gapmer approach. Thus, conventional antisense oligos with both potent steric hindrance and RNase H promoting activity can be generated and used for the practice of this invention.

The availability of antisense NABTs directed to the inhibition of the same target gene by different or overlapping inhibitory mechanisms allows for greater flexibility in treatment options for certain medical disorders. In cancer, for example, RNase H dependent NABTs can be used to attack the malignant stem and progenitor cells while sparing other cells in the cancer. If the success of the treatment requires the malignant stem and progenitor cells to be in cycle there can be an advantage to not attacking the other cells in the cancer because they can promote the proliferation of the malignant stem and progenitor cells. In other instances, rapidly debulking the tumor mass in a patient may be important. Here an antisense NABT with a steric hindrance mechanism would be the agent of choice since it will be operative on a much broader range of cancer cells. If the antisense NABT is intended to protect normal tissues from the toxic effects of conventional cytotoxic cancer therapeutics, then one with a combined RNase H and steric hindrance mechanism may be preferred so that the range of normal cell types is more broadly and thoroughly protected.

RNAi is suitable for the practice of this invention. Double stranded RNA of 25-30-mer length (dicer substrate) is cleaved intracellularly by the enzyme dicer to form approximately double stranded 21-mers with a two nucleotide (2-nt) overhang on each 3′ end. Such duplexes with the ability to selectively inhibit the expression of particular genes are referred to as siRNA. siRNA can cause specific gene inhibition in cells following loading into RISC and the discarding of one of the double strands (passenger strand). The RISC based mechanism of siRNA action is broadly expressed in cells where it is the same mechanism used for microRNA processing. MicroRNA is known to play a key role in regulating gene expression in all mammalian cell types. siRNA typically inhibits gene expression by targeting RNA transcripts of the gene in question for cleavage by an argonaute enzyme or by translational inhibition without RNA cleavage. siRNA can also directly inhibit gene expression by a mechanism that is not well defined and it can occur in a single stranded form that is distinguishable from conventional antisense oligos by its requirement for an argonaute enzyme for activity.

Adaptation of RNAi to pharmaceutical use includes the administration of NABTs that generally correspond to different components of the normal RNAi mechanisms. These are dicer substrates, siRNA (double stranded) and ss-siRNA (single stranded siRNA). As discussed more fully below, typical modifications used in the pharmaceutical variants of these molecules typically include backbone modifications to increase stability, base and/or other alterations to ensure that the desired strand will be chosen as the guide strand and the use of a carrier to transport the RNAi NABT into the cytoplasm of cells.

siRNA has the potential advantage of typically having a catalytic mechanism whereby the guide strand RISC complex causes cleavage of its target RNA and then goes on to cleave additional targets. Therefore, catalytic siRNA is potentially more active in a wider range of cell types than conventional antisense oligos that have an RNase H dependent mechanism. From this point of view, siRNA has a comparable range of cell types as conventional antisense with a steric hindrance mechanism. Conventional antisense oligos with an RNase H dependent mechanism, however, in principle can target anywhere on the pre-mRNA transcript because RNase H activity is usually limited to the nucleus. In contrast, siRNA dependent catalysis by an argonaute enzyme is usually limited to the cytoplasm and as a result the target sequences are limited to mature mRNA.

Existing RNAi based drugs have disadvantages that include the following: (1) The

RISC mechanism that is required for the functioning of an RNAi drug is also required for the processing of microRNAs that are essential for normal cellular function. Thus, there is the potential for competition between such RNAi based drugs and microRNA for processing that could result in serious side effects; and (2) Conventional RNAi drug design methods result in guide strands that have relatively modest binding affinities for their target sequences. Thus, they exhibit a lower efficiency of cleavage than could be obtained using higher affinity guide strands. Thus conventional RNAi drugs require greater dosage levels, which in turn increases their likelihood for interfering with microRNA processing. In contrast to the conventional approach, the present invention provides for RNAi NABTs with high affinity guide strands.

siRNA NABTs for the purposes of this invention will have an antisense or guide strand that are based on hot spot sequences provide in Table 8. The hot spots in the table are written as DNA sequences. When the NABT is an RNAi, the thymine (T) bases should be read as uracil (U) bases. Table 8 provides a list of all of the suitable size variants for the guide strands for each hot spot. The sequence of the passenger strand(s) forming a duplex with the guide strand can be determined on the basis of conventional base pairing A:U and G:C. In the case of 15-mers or 14-mers that are not explicitly listed in the table, it is only necessary to delete one or two nucleotides from the 3′ end of any given 16-mer to arrive at the indicated size. The prototype NABTs shown in this table were designed with conventional antisense mechanisms in mind and are suitable for this purpose.

siRNAs that function as transcriptional gene silencers range in size from 18-30mers and preferably contain sequences complementary to sequences within 150 bp of the transcriptional start site of the gene to be inhibited. Hot spots in Table 8 particularly preferred for down regulating expression of the p53 gene by targeting portions of SEQ ID NOs 1 and 2806-2815 or their complementary sequence including the corresponding size variants defined by Table 8 as well as sequences that are selected from an 16-30-mer guide strand based on the following sequence (SEQ ID NO: 3630) 5′-CAAAACUUUUAGCGCCAGUCUUGAGCA CAUGGGAGGGGAAAACCCCAAUC-3′ or its complement. Inosine may be substituted for one or two of the four sequential Gs to reduce any g-quartet effects if needed. The antisense sequences listed in Table 8 or their complementary sequences are suitable for NABTs that are transcriptional gene silencers because either of the two DNA sequences that make up particular genes can be targeted. Characteristics, delivery and production of siRNA transcriptional gene silencers are described in Lippman et al., Nature 431: 364, 2004; US2007/0104688.

siRNA NABTs can be administered to cells as dicer substrates for the purposes of this invention. In this instance, the guide strands selected from Table 8 will be 25-30mers. Once inside the cell, dicer will cleave the 3′ ends of the duplexed stands in a manor that leaves a two nucleotide (2-nt) overhang on the 3′ ends resulting in a potentially functional siRNA. A potential advantage of the administration of dicer substrates over their siRNA counterparts is that the former can be several fold more active in the subnanomolar concentration range. The design considerations for siRNA derived from dicer substrates is basically the same as what is described for administered siRNA with any needed allowances for dicer processing. Characteristics, chemical modifications and production of dicer substrates including their association with peptide carriers often but not necessarily as part of nanoparticles, nanocapsules, nanolattices, microparticles, micelles or liposomes (also see section on carriers below) are described in: Amarzguioui and Rossi, Methods Mol Biol 442: 3, 2008; Collingwood et al., Oligonucleotides 18: 187, 2008; Kim et al., Nature Biotech 23: 222, 2004; US2007/0265220, WO2007/056153, WO2008/022046.

For the purposes of this invention, the preferred length for siRNA other than dicer substrates or transcriptional gene silencers is a 16-mer duplex with a range of 14-25-mers with a two nucleotide (2-nt) overhang on the 3′ ends so that each preferred strand (guide or passenger) will consist of 18 nucleotides. The overhanging 2-nt are not necessarily required although are preferred and if present they are not typically required for the guide strand binding to its RNA target and consequently Us or Ts can be used as the overhanging bases irrespective of the target RNA sequence. The 5′ end of the guide strand of functional siRNA is phosphorylated. siRNA can be administered in this form or guide strand 5′ end phosphorylation may occur in cells as a result of the action of the Clp1 kinase.

For the purposes of this invention, the siRNA NABTs based on the hot spots in Table 8 will have two primary design considerations: (1) in the case of double stranded siRNAs, methods to bias loading of the RISC complex with the desired guide strand rather than the desired passenger strand; and (2) methods to stabilize siRNA NABTs in biological fluids without significantly reducing their activity against their RNA or DNA target. The methods for achieving the first objective fall into three main groups that are not mutually exclusive: (1) Blocking the 5′ end of the intended passenger strand, for example with an alkyl group, so that it cannot be phosphorylated by an intracellular kinase (Chen et al., RNA 14: 263, 2008); and/or (2) Using a nicked passenger strand, that is, one that is in effect two (preferably) or more strands that are contiguous when duplexed with the guide strand. In other words, unlike the passenger strands of typical siRNA, there is at least one missing linkage between adjacent nucleosides. Alternatively the passenger strand may have a gap where one or two nucleotides are missing with respect to the formation of a duplex with the guide strand; and/or (3) Selecting guide stands that have a lower Tm for the first 4-nt of their 5′ end as duplexed with the four duplexed nucleotides at the 3′ end of the passenger strand (leaving aside any 2-nt overhang) compared to the 5′ end of the corresponding passenger strand duplexed with the 3′ end of the guide strand (the opposite end of the duplex and leaving aside any 2-nt overhang). Alternatively modifying one or more nucleotides found in the four nucleotides at the 5′ end of the passenger stand to increase its Tm as a duplex with the 3′ end of the guide strand relative to the opposite end of the duplex or decrease the affinity of the four nucleotides at the 3′ end of the passenger stand for the 5′ end of the guide strand relative to the opposite end of the duplex can also be done. The methods for obtaining the second objective involve the use of several of the same types of modifications discussed in the section dealing with conventional antisense oligos. Hence many of the references for defining the synthesis methods and characteristics of the resulting oligos apply to the siRNA variants discussed herein.

In addition to promoting the loading of the complementary guide strand into RISC, discontinuous passenger strands increase the extent to which the nucleotides in the guide strand can be modified with the types of changes discussed herein for conventional antisense oligos (including but not limited to LNA, FANA, 2′ fluoro and piperazine) without significant loss of activity. The preferred siRNA with a discontinuous passenger strand has a single missing linkage between two nucleosides found within the central six nucleosides of the 16-mer duplex (total of 5 possible linkages any one of which can be eliminated). Further, the binding affinities of the two contiguous passenger strands for their guide strand partner should be at a Tm of least 42° C. The use of multiple LNA, FANA, 2′ fluoro and piperazine modified nucleosides can be used to boost the Tm and to stabilize the siRNA from nuclease attack, a topic discussed in more detail below. It is preferable, however, to have a lower Tm for the 5′ end of the guide stand duplexed with the 3′ end of the adjacent passenger strand as discussed elsewhere. Of these modifications LNA produces the highest increase in Tm with at least a several degree increase extending up to 10° C. being seen for each LNA nucleoside modification. Characteristics and production of siRNA with a discontinuous passenger strand is presented in: Bramsen et al., Nucleic Acids Res 35: 5886, 2007; WO2007/107162 and WO2008/049078.

The first four duplexed bases at the 5′ end of the desired guide strand, in descending order of importance starting with the terminal base, play an important role in determining which strand in duplexed siRNA will be loaded into the RISC complex as the guide strand. The Tm for this duplex is preferably lower that the Tm for the terminal four base duplex at the other end of the hybrid. This difference can be less than one degree centigrade but with such a small difference it is relatively more important that the two most terminal bases have a lower affinity compared to their counterparts at the other end of the duplex. Tms, including those for duplexes containing various mismatches, can be estimated using nearest neighbor calculations and experimentally determined more exactly using well established methods (Allawi et al., Biochem 36: 10581, 1997; Sugimoto et al., Biochem 25: 5755, 1986; Sugimoto et al., Biochem 26: 4559, 1987; Davis et al., Biochem 46: 13425, 2007; Freier et al., Proc Natl Acad Sci 83: 9373, 1986; Kierzek et al., Biochem 25: 7840, 1986; Freier et al., Biochem 25: 3209, 1986; Peyret et al., Biochem 38: 3468, 1999; Allawi et al., 37: 2170, 1998; Riccelli et al., Biochem 38: 11197, 1999; Bourdelat-Parks and Wartell, Biochem 44: 16710, 2005).

Table 8 provides for guide strands of lengths from 14-30-mer with 16-mers being preferred the passenger strand is simply the complement of the guide strand with possible overhangs and other possible modifications as described herein. If the first four duplexed bases at the 5′ end of the desired guide strand do not naturally have the relatively reduced Tm discussed above, then one or two base modifications of certain types can be made in the terminal four duplexed bases at the 3′ end of the passenger strand to provide the desired Tm reduction. Such base modifications can involve the introduction of mismatches between normal bases or the introduction of certain so-called “universal bases” which are defined as abnormal bases that can pair with at least two normal bases to form a nucleotide duplex (Hohjoh, FEBS Lett 557: 193, 2004). For the purposes of this invention, universal bases that may be incorporated into NABTs include but are not limited to hypoxanthine (inosine in ribonucleoside form), 5-nitroindole and 3-nitropyrrole. As an alternative to a universal base, a ribose moiety with no base at all can be used (abasic nucleoside) such as but not limited to the abasic spacer 1,2-dideoxyribose. Characteristics and production of oligos containing these and other universal bases and/or abasic sites are discussed in but not limited to the following: (Bergstrom et al., Nucleic Acids Res 25: 1935, 1997; Huang and Greenberg J Org Chem 73: 2695, 2008; Sagi et al., Biochem 40: 3859, 2001; Pompizi et al., Nucleic Acids Res 28: 2702, 2000; Loakes, Nucleic Acids Res 29: 2437, 2001; Watkins and SantaLucia, Nucleic Acids Res 33: 6258, 2005; Wright et al., Biochem 46: 4625, 2007; Loakes and Brown, Nucleic Acids Res 22: 4039, 1994; Van Aerschot et al., Nucleic Acids Res 23: 4363, 1995; Loakes et al., Nucleic Acids Res 23: 2361, 1995; Amosova et al., Nucleic Acids Res 25: 1930, 1997; Seio et al., J Biomol Struct & Dynam 22: 735, 2005; US2007/0254362, US2003/0171315, US2003/0060431, U.S. Pat. No. 6,600,028, U.S. Pat. No. 6,313,286, U.S. Pat. No. 5,438,131, WO2006/093526, WO99/06422, WO98/43991.

Methods to stabilize siRNA NABTs in biological fluids are essentially the same as those used for conventional antisense oligos, however, certain adjustments are needed to maintain compatibility with the endogenous RNAi and/or siRNA mechanisms that result in RISC loading and subsequent inhibition of target gene expression. A notable exception is the phosphorothioate modification commonly used in conventional antisense oligos to prevent nuclease attack because they do not similarly protect RNA analogs. Nevertheless phosphorothioate linkages can be useful components of RNAi drugs because they promote binding to plasma proteins such as albumin and thus may improve tissue distribution and uptake.

Generally, most modifications to the passenger strand derived from the guide strand sequences provided in Table 8 will not negatively influence siRNA function typically as long as the duplex retains its A-form-like helical structure. These include the numerous possible modifications at the 2′ position of the pentose sugar that are well tolerated by the siRNA mechanisms and further discussed herein. Such modifications include but are not limited to the addition of a 2′ fluorine atom (2′-fluoro) to the furanose ring in nucleosides in one or more of the passenger or guide strands. Further using nucleosides with alternating 2′-O-methyl with 2′-fluoro modifications or alternating 2′-O-methyl with normal ribose containing nucleotides where the 2′-O-methyl preferably starts at the 5′ terminal nucleoside of the guide strand and is paired to a nucleoside in the passenger strand that does not have a 2′-O-methyl also are suitable for use in the present invention.

Additional 2′-O-methyl modifications that are suitable for use in this invention include but are not limited to the following guide stand modifications paired with a fully 2′-O-methyl modified passenger strand: (1) 2′-O-methyl modifications to the final two 3′ end duplexed nucleosides; (2) the insertion of 2′ fluoro containing nucleosides at the opposite one-third ends of the strand while avoiding the center one-third (for example, avoid the center 6 nucleosides in a 16-mer duplex with 2-nt overhang) preferably where at least two such modifications occur in the 5′ one-third of the nucleosides and in all of the 3′ one-third; (3) fully phosphorylated with or without the 2′-O-methyl or 2′-fluoro modifications just described. Characteristics of siRNA with 2′-O-methyl or 2′-O-methyl and 2′-fluoro modifications are discussed in but not limited to the following: Allerson et al., J Med Chem 48: 901, 2005; Layzer et al., RNA 10: 766, 2004; WO2004/043977 and WO2004/044133, WO2005/121370, WO2004/043978, WO2005/120230, WO2007/0004665. siRNA that is fully 2′ fluoro substituted is also suitable for the practice of this invention. Characteristics and production of such siRNA is described by Blidner et al., Chem Biol Drug Des 70: 113, 2007.

LNA modifications suitable for the practice of this invention include but are not limited to the insertion of LNA nucleosides in each of the passenger and guide strands at the opposite one-third ends of the strands that avoid the center one-third (for example, avoid the center 6 nucleosides in a 16-mer duplex with 2-nt overhang) and which also respect the rules described herein that deal with the desirability of having a lower Tm for the duplex at the 5′ end of the guide stand compared to the 5′ end of the passenger strand. Particularly in the case of siRNAs with a discontinuous passenger strand as additional LNA substitutes in these regions are to be preferred. Characteristics of siRNA with LNA modifications are discussed in but not limited to the following: Elmen et al., Nucleic Acids Res 33: 439, 2005; US 2007/0004665, US 2007/0191294, WO2005/073378, WO2007/085485.

FANA modifications suitable for the practice of this invention include but are not limited to the insertion of FANA nucleosides in one or more of the passenger strand nucleosides and at the opposite one-third ends of the guide strand avoiding the center one-third (for example, avoid the center 6 nucleosides in a 16-mer duplex with 2-nt overhang) and which also respect the rules described herein that deal with the desirability of having a lower Tm for the duplex at the 5′ end of the guide stand compared to the 5′ end of the passenger strand. Particularly in the case of siRNAs with a discontinuous passenger strand, larger numbers of FANA substitutes are to be preferred. Characteristics of siRNA with FANA modifications are discussed in but not limited to the following: Dowler et al., Nucleic Acids Res 34: 1669, 2006; WO2007/048244.

Alternatively, each of the 2′-O-methyl, LNA or FANA modifications just described can be replaced with nucleosides where a piperazine ring has replaced the furanose to produce antisense NABTs that include those based on sequences in Table 8. In addition to increasing nuclease resistance and improving specific target binding, the piperazine modification is less likely to produce oligos (including but not limited to those configured as a siRNA duplex) that stimulate immune responses such as those mediated by interferon and/or are mediated by toll-like receptors.

In the case of expression vectors, those suitable for the practice of this invention will produce within target cells antisense sequences that include one or more of the hot spots provided in Table 8 for the gene to be targeted. Preferably, such expression vectors will produce a transcript that includes, but is not limited to an entire hot spot. Such expression vectors may be designed to integrate into the genome of target cells or to function extrachromosomally. In general, integrated vectors are preferred in instances where very long-term target gene suppression is preferable. Integration, however, can infrequently produce alterations in endogenous genes that may become pathogenic. Accordingly, it is generally preferable to not use an expression vector of this type to suppress gene expression in stem cells unless the stem cells are critical to a fatal disease and there is a need for prolonged suppression for therapeutic purposes. Thus, in general it will be preferable to use a non-integrating expression vector when the commercial goal includes suppressing the expression of a particular gene in stem cells. Characteristics and production methods for expression vectors appropriate for use in the present invention include but are not limited to those described in the following: Adriaansen et al. Rheumatology 45: 656, 2006; Vinge et al., Circ Res 102: 1458, 2008; Lyon et al., Heart 94: 89, 2008; Buch et al., Gene Ther 15: 849, 2008; Zentilin and Giacca, Contrib Nephrol 159: 63, 2008; Wang and Pham, Expert Opin Drug Deliv 5: 385, 2008; Mandel et al., Mol Ther 13: 463, 2006; Kordower and Olanow, Exp Neurol 209: 34, 2008; Muller et al., Cardiovasc Res 73: 453, 2006; Warrington and Herzog, Hum Genet 119: 571, 2006; U.S. Pat. No. 7,393,526, U.S. Pat. No. 7,402,308, U.S. Pat. No. 6,309,634, U.S. Pat. No. 6,436,708, U.S. Pat. No. 6,830,920, U.S. Pat. No. 6,174,871, U.S. Pat. No. 6,989,374, U.S. Pat. No. 6,867,196, U.S. Pat. No. 7,399,750, U.S. Pat. No. 6,306,830, U.S. Pat. No. 5,770,580, U.S. Pat. No. 7,175,840, US20070104687, U.S. Pat. No. 7,312,324, U.S. Pat. No. 7,211,248, U.S. Pat. No. 7,001,760, U.S. Pat. No. 5,895,759, WO05021768, WO9506745.

In addition to viral vectors, many of the carrier mechanisms being applied to siRNA and dicer substrates that are presented herein have their origins as carriers for the transfer of genetically engineered genes into cells in vitro as well as in vivo and are useful for introducing nucleic acids encoding antisense molecules based on the sequences provided in Table 8 into cells where the gene will cause the antisense transcript to be produced.

When choosing an NABT of the invention for treatment of a pathological disorder, certain factors should be considered. These include: (1) the differentiation stage of the cells containing the gene to be inhibited by the NABT; (2) the desired duration of the NABT therapeutic effect; (3) the function of the specific target sequence in the RNA transcript of the gene to be inhibited; (4) the relative concentration of the NABT in the nuclear and cytoplasmic compartments; and (5) the nature of the desired therapeutic or other commercial use effect. Tables 15, 16 and 17 and the following discussion provide a summary of some of the considerations that can be used to guide NABT selections.

There is significant overlap between the capabilities of the different types of NABT and, therefore, more than one NABT type can work for any given purpose. The single most important aspect of any NABT is the sequence of its antisense or guide strand and all of the hot spot sequences provided by Table 8 as described herein can be used to generate antisense or guide strand sequences for NABTs with mechanisms involving RNase H, RISC or steric hindrance by expression vectors. The prototype sequences are preferred for use in conventional antisense oligos. Several of these and their hotspots show superior properties and act via a steric hindrance mechanism as described herein.

In general, the most efficient NABTs are those with RNase H activity, assuming the target cells have sufficient RNase H activity to support their antisense activity. Preferred NABTs for this purpose are shown in Table 15. The reasons for the relatively high efficiency are the following: (1) such NABTs, in the presence of RNase H have catalytic activity leading to the degradation of multiple RNA targets by a single NABT; and (2) conventional antisense oligos do not typically require a carrier for in vitro use unlike dicer substrates or siRNA and as a result uptake into cells is more efficient.

All of the hotspots and prototypes shown in Table 8 provide suitable sequences for use in conventional antisense oligos with RNase H activity. Adequate RNase H activity is reliably present in stem cells and early (that is early in expressing their differentiation program) progenitor cells while it is uncommon in other cell types. Accordingly, obtaining broader activity than stem cells and early progenitors with respect to the differentiation status of the target cells depends on the use of an NABT with a steric hindrance or RISC dependent mechanism (Tables 15-17).

Different types of NABT also can be roughly distinguished on the basis of how long they act in cells. Conventional antisense oligos tend to be shorter acting (days to 2-3 weeks) compared to dicer substrates or siRNA (about a month) that in turn are shorter acting than expression vectors (months or even years). With the exception of certain expression vectors that get duplicated during cell division, NABTs are not duplicated by cells so they are degraded and/or in the case of cells that divide, diluted out over time.

NABTs that affect cellular programming can also impact the duration of their effect on cells as a consequence of their biologic effects. NABTs that promote apoptosis, for example, will have a very short period of action because they kill the cells in which they produce their therapeutic effect. NABTs that promote cellular differentiation that have an RNase H mechanism of action can lose their action on cells by causing them to differentiate and concomitantly loose RNase H activity.

Thus, NABT type selection is dependent on the therapeutic or other commercial use to which the NABT is to be put. Cancer, for example, is maintained by stem cells and/or early progenitor cells. Further, the desired therapeutic end point is to kill these cells. It follows, therefore, that conventional antisense oligos that support RNase H activity are particularly well suited for treating cancer. If it is desirable to rapidly debulk a cancer then conventional antisense oligos that also have a steric hindrance mechanism may be preferable because they will work in a much broader range of the malignant cells in a given cancer. So it can be anticipated that in some applications that more than one NABT might be required to obtain the best outcome. In contrast to cancer, treatments to block apoptosis in certain chronic diseases, for example, such congestive heart failure or prophylactically protecting tissues from ischemia reperfusion injury typically are better served by longer acting NABTs such as dicer substrates, siRNA or expression vectors compared to conventional antisense oligos.

The two main subcellular compartments where NABTs carry out their gene inhibitory effects are the nucleus and/or the cytoplasm. Thus, in certain instances it may be desirable to compare the relative levels of any given NABT in these two compartments relative to the site of action of the NABT (Tables 15-17). Other considerations being equal it is important to choose an NABT that preferentially accumulates in the subcellular compartment appropriate to its mechanism. As provided herein there are certain carrier modifications that can direct associated NABTs to particular subcellular compartments as needed.

In addition, modified NABT backbones suitable for use in the present invention include, for example, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. NABTs having inverted polarity comprise a single 3′ to 3′ linkage at the 3′-most internucleotide linkage i.e., a single inverted nucleoside residue which may be abasic (the base is missing or has a hydroxyl group in place thereof) are suitable for use in the present invention. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

Additional modified NABT backbones suitable for use in the present invention that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In other NABTs suitable for use in the present invention both the sugar and the internucleoside linkage, i.e., the backbone, of the nucleotide units are replaced with novel groups. The base units are maintained for hybridization with an appropriate nucleic acid target compound. One such oligo compound, an NABT mimetic that has been shown to have excellent hybridization properties, is referred to as a peptide nucleic acid (PNA). In PNA compounds, the sugar-backbone of an NABT is replaced with an amide containing backbone, in particular an aminoethylglycine backbone. The bases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500. Suitable NABTs with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240.

Suitable modified NABTs may also contain one or more substituted sugar moieties. Such NABTs may comprise one of the following at the 2′ position: OH; O—, S—, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O[(CH2)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other suitable NABTs comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an NABT, or a group for improving the pharmacodynamic properties of an NABT, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other suitable modifications include 2′-methoxy (2′-O—CH₃), 2′-aminopropoxy (2′-OCH₂CH₂CH₂NH₂) and 2′-allyl (2′—CH₂—CH═CH₂), 2′—O-allyl (2′-O—CH₂—CH═CH2). Modifications to the sugar may be in the arabino (up) position or ribo (down) position and may be made at various positions on the sugar, particularly the 3′ position of the sugar on the 3′ terminal nucleotide or in 2′-5′ linked sugars and the 5′ position of 5′ terminal nucleotide sugar. Suitable NABTs may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

Suitable NABTs may also include nucleobase (often referred to in the art simply as “base”) modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C—C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-][1,4]benzoxazin-2(3H)-one), phenothiazine cytidine (1H-pyrimido[5,4-][1,4]benzothiazin-2(3H)-one), G-clamps such as a substituted phenoxazine cytidine (e.g. 9-(2-aminoethoxy)-H-pyrimido[5,4-][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these bases are particularly useful for increasing the binding affinity of the oligo compounds of the invention. These include 5-substituted pyrimidines, 6-azapyrimidines and N2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are suitable base substitutions, even more particularly when combined with 2′-O-methoxyethyl sugar modifications.

Representative United States patents that teach the preparation of certain of the above noted modified bases as well as other modified bases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941, and 5,750,692, each of which is herein incorporated by reference.

Another modification of the NABTs of the invention involves chemically linking to the NABT one or more moieties or conjugates that enhance the activity, cellular distribution or cellular uptake of the NABT. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligos, and groups that enhance the pharmacokinetic properties of oligos. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligo uptake, enhance oligo resistance to degradation, and/or strengthen sequence-specific hybridization with RNA. Groups that enhance the pharmacokinetic properties, in the context of this invention, include groups that improve oligo uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. NABTs of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. Pat. No. 6,656,730 that is incorporated herein by reference in its entirety.

Representative United States patents that teach the preparation of such NABT conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an NABT. The present invention also includes antisense compounds that are chimeric compounds. “Chimeric” antisense compounds or “chimeras,” in the context of this invention, are antisense compounds, particularly NABTs, which contain two or more chemically distinct regions, each made up of at least one monomer unit, i.e., a nucleotide in the case of an NABT compound. These NABTs typically contain at least one region wherein the NABT is modified so as to confer upon the NABT increased resistance to nuclease degradation, increased cellular uptake, and/or increased binding affinity for the target nucleic acid. An additional region of the NABT may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of NABT inhibition of gene expression. Consequently, comparable results can often be obtained with shorter NABTs when chimeric NABTs are used, compared to phosphorothioate deoxyoligos hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more NABTs, modified NABTs and/or NABT mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The NABTs used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare NABTs such as the phosphorothioates and alkylated derivatives.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The NABTs of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, prodrugs and pharmaceutically acceptable salts of the compounds of the invention, pharmaceutically acceptable salts of such prodrugs, and other bioequivalents are also encompassed by the present invention. In addition, conventional antisense NABTs may be formulated for oral delivery (Tillman et al., J Pharm Sci 97: 225, 2008; Raoof et al., J Pharm Sci 93: 1431, 2004; Raoof et al., Eur J Pharm Sci 17: 131, 2002; U.S. Pat. No. 6,747,014; US 2003/0040497; US 2003/0083286; US 2003/0124196; US 2003/0176379; US 2004/0229831; US 2005/0196443; US 2007/0004668; US 2007/0249551; WO 02/092616; WO 03/017940; WO 03/018134; WO 99/60012). Such formulations may incorporate one or more permeability enhancers such as sodium caprate that may be incorporated into an enteric-coated dosage form with the NABT.

For example, where a NABT is to be expressed, the antisense strand may be operatively linked to a suitable promoter element, for example, but not limited to, the cytomegalovirus immediate early promoter, the Rous sarcoma virus long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells; one example of such a promoter is the PEG-3 promoter, as described in International Patent Application No. PCT/US99/07199, Publication No. WO 99/49898 (published in English on Oct. 7, 1999); other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takalcuwa et al., 1997, Jpn. J. Cancer Res. 88:166-175), the human carcinoembryonic antigen gene promoter (Lan et al., 1996, Gastroenterol. 111:1241-1251), the gastrin-releasing peptide gene promoter (Inase et al., 2000, Int. J. Cancer 85:716-719). the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535), which is particularly active in pancreatic cancer cells (Perrais et al., 2001, J. Biol Chem. 276(33):30923-33).

Suitable expression vectors include virus-based vectors and non-virus based DNA or RNA delivery systems. Examples of appropriate virus-based gene transfer vectors include, but are not limited to, those derived from retroviruses, for example Moloney murine leulcemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2): 113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, “naked” nucleic acids (Wolff et al., 1990, Science 247:1465-1468), nucleic acids encapsulated in liposomes (Nicolau et al., 1987, Methods in Enzymology 1987:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

Oligos may also be produced by yeast or bacterial expression systems. For example, bacterial expression may be achieved using plasmids such as pCEP4 (Invitrogen, San Diego, Calif.), pMAMneo (Clontech, Palo Alto, Calif.; see below), pcDNA3.1 (Invitrogen, San Diego, Calif.), etc.

Examples of methods of gene expression analysis useful in conjunction with the present invention are well known in the art (Measuring Gene Expression (2006) M Avison, Taylor & Francis; Advanced Analysis of Gene Expression Microarray Data (2006) A Zhang, World Scientific Publishing Company) and include DNA arrays or microarrays (Brazma and Vilo, FEBS Lett 480: 17, 2000; Celis, et al., FEBS Lett 480: 2, 2000), SAGE (serial analysis of gene expression) (Madden, et al., Drug Discov. Today, 5: 415, 2000), READS (restriction enzyme amplification of digested cDNAs) (Prashar and Weissman, Methods Enzymol. 303: 258, 1999), TOGA (total gene expression analysis) (Sutcliffe, et al., Proc. Natl. Acad. Sci. U.S. A. 97: 1976, 2000), protein arrays and proteomics (Celis, et al., FEBS Lett 480: 2, 2000; Jungblut, et al., Electrophoresis 20: 2100, 1999), expressed sequence tag (EST) sequencing (Celis, et al., FEBS Lett. 480: 2, 2000; Larsson, et al., J. Biotechnol. 80: 143, 2000), subtractive RNA fingerprinting (SuRF) (Fuchs, et al., Anal. Biochem 286: 91, 2000; Larson, et al., Cytometry 41: 203, 2000), subtractive cloning, differential display (DD) (Jurecic and Belmont, Curr Opin Microbiol 3: 316, 2000), comparative genomic hybridization (Carulli, et al., J Cell Biochem Suppl. 31: 286, 1998), FISH (fluorescent in situ hybridization) techniques (Going and Gusterson, Eur J Cancer 35: 1895, 1999) and mass spectrometry methods (reviewed in (To, Comb Chem High Throughput Screen 3: 235, 2000).

When systemically administered without the use of a carrier, antisense NABTs including conventional antisense oligos, dicer substrates and siRNA, but not expression vectors, have a similar distribution pattern to major organs in the body with liver and kidney taking up the most of these materials and the CNS the least. At subtoxic doses, conventional antisense oligos can be detected in all major tissues including the brain following systemic administration. Further, animal models involving a wide range of targets and tissue types have shown that conventional antisense oligos with variable mechanisms of action (for example RNase H dependence and/or one of various types of steric hindrance) and a variety of backbone chemistries have demonstrable antisense effects against their intended target in vivo when delivered without a carrier. In contrast to conventional antisense oligos, dicer substrates, siRNA and expression vectors typically require the use of a carrier to get them into cells in vivo in the amounts needed for their intended antisense effects. Exceptions for dicer substrates and siRNA may include liver and kidney as well as local administration to sequestered sites such as the eye where the NABT can be retained for a prolonged period.

Cationic liposomal carriers are often employed in vitro to transfer NABTs including conventional antisense oligos into cell lines to reduce sequestration of naked antisense NABTs in endosomes and certain other intracellular vesicles, thereby increasing the availability of the NABT to bind to the desired target within the cell. Endosomal sequestration of NABTs, however, does occur albeit to a lower degree in vivo.

There are a number of strategies for increasing the efficiency of conventional antisense oligos in vivo that allow for dose reductions and/or for a given dose to be effective for a longer period of time. Such oligos, for example, are more efficiently delivered to intracellular compartments and appear to exhibit higher activity when they are concatemerized into complexes such as those described by Simonova et al., in Biochim Biophys Acta 1758, 413, (2006); and Gusachenko et al., in Human Gene Ther 19: 532, (2008). This concatemerization can be achieved, in part, by the use of a carrier oligo that binds to the conventional antisense oligo by complementary base pairing. In one embodiment, the ends of the duplex have short over hangs and the carrier oligo optionally includes one or more lipophilic group(s) and/or other groups capable of improving membrane penetration. This enhanced penetration also can be achieved by covalently attaching the lipophilic group(s) (e.g., cholesterol) to the oligo. Alternatively, the lipophilic group can be attached to a “double stranded stopper oligo” with over hangs, one overhang of which binds to the antisense/carrier oligo complex by complementary base pairing while the other strand has the lipophilic group covalently attached to it. In a variant embodiment, the binding affinity of the carrier oligo for the antisense oligo is reduced by means of incorporating mismatches, abasic nucleosides or universal bases (as described elsewhere herein) as necessary to reduce the Tm of the duplex to less than 55° C. when measured under conditions of physiological salt concentrations and pH. These and alternatives to this approach that do not involve the covalent attachment of molecule(s) capable of promoting membrane penetration to the carrier oligo are applicable also to the delivery of dicer substrates or siRNA and are described in the documents provided.

Packaging RNA (pRNA) can be incorporated into a plurality of chimeric complexes each carrying at least one NABT and used to deliver said NABT to cellular compartments such as the cytoplasm or nucleus where said NABT can perform its intended antisense function. Characteristics, production, methods and uses of pRNA complexes that are suitable for use with the present invention are presented in but not limited to the following: Guo, Methods Mol Biol 300: 285, 2005, Guo, J Nanosci Nanotechnol 5: 1964, 2005; and WO 2007/016507.

There are also delivery mechanisms applicable to NABTs with or without carriers that can be applied to particular parts of the body such as the CNS. These include the use of convection-enhanced delivery methods such as but not limited to intracerebral clysis (convection-enhanced microinfusion into the brain—Jeffrey et al., Neurosurgery 46: 683, 2000) to help deliver the cell-permeable carrier/NABT complex to the target cells in the CNS as described in WO 2008/033285.

Drug delivery mechanisms based on the exploitation of so-called leverage-mediated uptake mechanisms are also suitable for the practice of this invention (Schmidt and Theopold, Bioessays 26: 1344, 2004). These mechanisms involve targeting by means of soluble adhesion molecules (SAMs) such as tetrameric lectins, cross-linked membrane-anchored molecules (MARMs) around lipoproteins or bulky hinge molecules leveraging MARMs to cause a local inversion of the cell membrane curvature and formation of an internal endosome, lysosome or phagosome. More specifically leverage-mediated uptake involves lateral clustering of MARMs by SAMs thus generating the configurational energy that can drive the reaction towards internalization of the NABT carrying complex by the cell. These compositions, methods, uses and means of production are provided in WO 2005/074966.

The various carriers contemplated for use in accordance with the present invention are divided into various categories below, but it is to be understood by the one skilled in the art that some components of these carriers can be mixed and matched. For example, various linkers can be used to attach various peptides of the type described herein to any given NABT and various peptides can be incorporated into particular nanoparticle-based carriers depending on the commercial or clinical purpose to be served.

Carriers and/or endosomolytic agents can be used to advantage for delivering adequate amounts of conventional antisense oligos and other types of NABTs in vitro or in vivo to certain intracellular compartments such as the nucleus or the cytoplasm and/or in delivering adequate amounts of such agents in vivo to certain tissues such as the following: (1) delivery to the brain, an organ that typically takes up relatively small amounts of NABTs following systemic administration; (2) preferentially concentrating NABTs in particular target organs, such as heart; and (3) increasing the levels of active NABTs in tissues more resistant to NABT uptake due to certain conditions, such as poor vascularization in tumors and disrupted blood supply in ischemia reperfusion injuries; and (4) reducing the dose needed for NABT action, while reducing potential side effect risk(s) in non-target tissues.

For the purposes of this invention, the preferred carriers, particularly for in vivo use, make use of peptides that promote cell penetration. These cell penetrating peptides (CPPs) typically share a high density of basic charges and are approximately 10-30 amino acids in length. Such peptides may be part of a complex carrier composition, including but not limited to nanoparticles. Alternatively, such CPP peptides may be conjugated to the NABT directly or by means of a linker. Further, CPPs can be fused to, or otherwise associated with peptides that provide other features to NABT carriers such as increasing homing to particular organs, or to particular subcellular compartments. For example, certain peptides described herein may enhance nuclear localization or provide an endosomolytic function (i.e., they function to enhance the escape of NABTs or other drugs from endosomes, lysosomes or phagosomes). CPPs and peptides with other useful carrier functions may be derived from naturally occurring protein domains or synthetic versions may be designed which retain the activity of the naturally occurring versions. Those of human origin include peptide-mimetics such as polyethylenimines. The naturally occurring peptides discussed below have sequence variants, such as those observed in different strains or species or as a result of polymorphisms within species. Thus, the representative peptide sequences provided cannot be considered to be exact and variations in peptide sequences exist between some of the documents referenced. These variants are fully functional and may be used interchangeably.

Given the relatively small size of most cell penetrating peptides compared to the large size of siRNA, dicer substrates or expression vectors, it is often preferable to employ such peptides in larger carrier structures such as nanoparticles rather than use direct conjugation of the peptide to these NABT types. This approach typically improves the charge ratio and cellular uptake for NABT/carrier complexes. However, an example of a CPP that has been directly and covalently attached to siRNA and shown to promote its uptake by cells is TAT (Chiu et al., Chem Biol 11: 1165, 2004; Davidson et al., J Neurosci 24: 10040, 2004). Delivery of antisense NABTs contained within expression vectors generally will require a viral vector or one of the siRNA or dicer substrate delivery mechanisms as provided for herein.

Targeting molecules may be operably linked to CPPs thus providing improved NABT uptake in particular cell types. One example of targeting molecules useful for this purpose are those directed to G-protein coupled receptors. Other examples of targeting molecules are ligands to IL-13, GM-CSF, VEGF and CD-20. Other examples of complex structures involved in targeting include nucleic acid aptamers or spiegelmers directed to particular cell surface structures. Characteristics, production uses and methods related to these targeting molecules and complex structures are provided in the following documents: (Nolte et al., Nat Biotech 14: 1116, 1996; McGown et al., Anal Chem 67: 663A, 1995; Pestourie et al., Biochimie 87: 921, 2005; Brody and Gold, J Biotechnol 74: 5, 2000; Mayer and Jenne, BioDrugs 18: 351, 2004; Wolfl and Diekmann, J Biotechnol 74: 3, 2000; Ferreira et al., Tumour Biol 27: 289, 2006; Stoltenburg et al., Anal Bioanal Chem 383: 83, 2005; Rimmele, Chembiochem 4: 963, 2003; Ulrich Handb Exp Pharmacol 173: 305, 2006; Drabovich et al., Anal Chem 78: 3171, 2006; Eulberg and Klussmann, Chembiochem 4: 979, 2003; Vater and Klussmann, Curr Opin Drug Discov Devel 6: 253, 2003; Binkley et al., Nucleic Acids Res 23: 3198, 1995; U.S. Pat. No. 7,329,638, US 2005/0042753, US 2003/0148449, US 2002/0076755, US 2006/0166274, US 2007/0179090, WO 01/81408, WO 2006/052723, WO 2007/137117, WO 03/094973, WO 2007/048019, WO 2007/016507, WO 2008/039173).

Methods and agents that can be used to bypass endosomal, lysosomal or phagosomal sequestration or used to promote the escape of NABTs from endosomes, lysosomes or phagosomes are optionally administered with the NABT based therapeutics described herein. Such methods include, but are not limited to three approaches that are not mutually exclusive. First, endosomolytic or lysosomotropic agents may be attached to NABTs or included in NABT carrier compositions. Second, lysosomotropic agents may be administered as separate agents at about the time the NABT or carrier/NABT complex is administered in vivo or in vitro. Such lysosomotropic agents include, but are not limited to, the following agents: chloroquine, omeprazole and bafilomycin A. Third, agents that inhibit vacuolar proton ATPase activity (promotes acidification of endosomes, lysosomes or phagosomes) or acidic organelle function may be utilized to sensitize cells to NABT action. Such agents and methods for their administration are provided in U.S. Pat. No. 6,982,252 and WO 03/047350. Such compounds include but are not limited to the following: (1) a bafilomycin such as bafilomycin A1; (2) a macrolide antibiotic such as concanamycin; (3) a benzolacton enamide such as salicilyhalamide A, oximidine or lobatamide; (4) inhibitors of rapamycin, bFGF, TNF-alpha, and/or PMA activated pathways; (5) inhibitors of the class III phosphatidylinositol 3′-kinase signal transduction pathway; and/or (6) antisense NABTs directed to the gene or RNA encoding vacuolar proton ATPase protein.

Certain lysosomotropic agents such as chloroquine and omeprazole have been used medically, but not as agents for the promotion of NABT activity. These agents exhibit lysosomotropic activity at established doses and treatment regimens both in vivo and in vitro, and thus such studies provide a dosing guide for their use in combination with NABTs to promote NABT activity (Goodman & Gilman's The Pharmacologic Basis of Therapeutics 11^{th} edition Brunton et al., editors, 2006, McGraw-Hill, New York). Other lysosomotropic agents are suitable for in vitro use and dosing studies can be performed according to well established methods known in the art to optimize efficacy when used in combination with NABT therapeutics in vivo. Methods have also been devised that allow chloroquine to be incorporated into carriers or directly conjugated to NABTs for boosting the intended antisense activity of NABTs on cells. These include but are not limited to, those found in US 2008/0051323 and WO2007/040469.

The molecules listed below are useful as carriers and/or as components of complex carriers for transporting the NABTs of the present invention into cells and into subcellular compartments (in accordance with the guidance provided herein) where they can express their antisense function. Unless otherwise noted these molecules: (1) are CPPs; and/or (2) are useful for achieving NABT function in a wide variety of cell types. Certain of the molecules have been shown to work well in particular cell types or tissues and/or to selectively work with particular cell types or tissues. Such tissues and cell types for which certain of the following molecules have proved to be particularly useful as targeting ligands, carriers or as members of complex carriers include but are not limited to brain, CNS, liver, heart, endothelium, pancreatic islet cells, retina, etc. The biochemical features of the following disclosed peptides and other molecules listed (e.g., increased target cell membrane penetration activity, promotion of endosomolytic activity, activation by to exposure to low pH environments and coding sequence information) are provided in detail below.

(1) TAT and TAT variants—See the following references: (Astriab-Fisher et al., Pharmaceutical Res 19: 744, 2002; Zhao and Weissleder, Med Res Rev 24: 1, 2004; Jensen et al., J Controlled Release 87: 89, 2003; Hudecz et al., Med Res Rev 25: 679, 2005; Meade et al., Adv Drug Delivery Rev 59: 134, 2007; Meade and Dowdy Adv Drug Delivery Rev 60: 530, 2008; Jones et al., Br J Pharmacol 145: 1093, 2005; Gupta et al., Oncology Res 16: 351, 2007; Kim et al., Biochimie 87: 481, 2005; Klein et al., Cell Transplantation 14: 241, 2005; U.S. Pat. No. 6,316,003, U.S. Pat. No. 7,329,638, US 2005/0042753, US 2007/0105775, US 2006/0159619, WO 99/55899, WO 2007/095152, WO 2008/008476, WO 2006/029078, WO 2006/0222657, WO 2008/022046, WO 2006/053683, WO 2004/048545, WO 2008/093982, WO 94/04686)—Tat includes the HIV TAT protein transduction domain and sequences that have been used for this purpose, such as: KRRQRRR (SEQ ID NO: 3631), GYGRKKRRQRRR (SEQ ID NO:3632), YGRKKRRQRRR (SEQ ID NO: 3633), CYGRKKRRQRRR (SEQ ID NO:3634), RKKRRQRRRPPQC (SEQ ID NO: 3635), CYQRKKRRQRRR (SEQ ID NO: 3636) and RKKRRQRRR (SEQ ID NO: 3637). In addition, various amino acid substitutions in TAT have been shown to promote the CPP activity of TAT as disclosed in the referenced documents. TAT can be used as a fusion peptide with enhanced CPP activity where the fusion partner is selected from peptides derived from the following group: (a) HEF from influenza C virus; (b) HA2 and its analogs, see below; (c) transmembrane glycoproteins from filovirus, rabies virus, vesicular stomatitis virus or Semliki Forest virus; (d) fusion polypeptide of sendai virus, human respiratory syncytial virus, measles virus, Newcastle disease virus, visna virus, murine leukemia virus, human T-cell leukemia virus, simian immunodeficiency virus; or (e) M2 protein of influenza A virus.

TAT and TAT variants have been used successfully to facilitate delivery of therapeutic agents to a wide variety of tissue and cell types that include but are not limited to the following: (a) the CNS and increase penetration of the blood brain barrier. See Kilic et al., Stroke 34: 1304, 2003; Kilic et al., Ann Neurol 52: 617, 2002; Kilic et al., Front Biosci 11: 1716, 2006; Schwarze et al., Science 285, 1569, 1999; Banks et al., Exp Neurol 193: 218, 2005; and WO 00/62067; (b) TAT peptides have also been shown to effectively penetrate heart tissue. See Gustafsson et al., Circulation 106: 735, 2002; (c) TAT or TAT/PDT are described in Embury et al., Diabetes 50: 1706, 2001; and Klein et al., Cell Transplantation 14: 241, 2005. These investigators disclose that such peptides are useful for delivery of desired agents to pancreatic islet cells; (d) Schorderet et al., Clin Exp Ophthalmology 33: 628, 2005 describe the use of D-TAT which is the retro-inverso form of TAT for delivery of agents to the retina and thus this peptide is also useful in the methods disclosed herein.

(2) MPG peptide—See the following references. (Morris et al., Nucleic Acids Res 25: 2730, 1997; Simeoni et al., Nucleic Acids Res 31: 2117, 2003; Hudecz et al., Med Res Rev 25: 679, 2005; Deshayes et al., Adv Drug Delivery Rev 60: 537, 2008; WO 2006/053683, WO 2004/048545)—Delivery systems using this CPP make combined use of a sequence that is derived from the fusion sequence of the HIV protein gp41, the sequence including for example, GALFLGF(or W)LGAAGSTMGA (SEQ ID NO:3638) or the longer peptide sequence GALFLGF(or W)LGAAGSTMGAWSQPKKKRKV (SEQ ID NO:3639) when the goal is to achieve higher levels nuclear transport of the NABT. Nuclear concentration is most suitable for conventional antisense oligos that have an RNase H mechanism of action or those that interfere with splicing by means of a steric hindrance mechanism as well as for siRNA that functions as a transcriptional inhibitor and for expression vectors. An alternative form of the longer MPG peptide where the second lysine is replaced by a serine (GALFLGF(or W)LGAAGSTMGAWSQPKSKRKV; (SEQ ID NO: 3640) causes the transported NABT to preferentially localize in the cytoplasm. This is most suitable for conventional antisense oligos that interfere with translation by a steric hindrance mechanism or for siRNA that function via interfering with translation, as well as for most dicer substrates or siRNA. In the MPG delivery system, these peptides are incorporated into nanoparticles that combine with NABTs by charge/charge interaction.
(3) Penetratin and EB1—See the following references. (Astriab-Fisher et al., Pharmaceutical Res 19: 744, 2002; Hudecz et al., Med Res Rev 25: 679, 2005; Lindgren et al., Bioconjugate Chem 11: 619, 2000; Meade et al., Adv Drug Delivery Rev 59: 134, 2007; Meade and Dowdy Adv Drug Delivery Rev 60: 530, 2008; Jones et al., Br J Pharmacol 145: 1093, 2005; Lundberg et al., FASEB J 21: 2664, 2007; U.S. Pat. No. 7,329,638, US 2005/0042753, US 2007/0105775, WO 2007/095152, WO 2008/008476, WO 2006/029078, WO 2006/0222657, WO2008/022046, WO 2006/053683, WO 2004/048545, WO 2008/093982)—Penetratin sequences include but are not limited to the following: RQIKIWFQNRRMKWKK (SEQ ID NO: 3641) and RQIKIWFQNRRMKWKKGGC (SEQ ID NO:3642). EB1 which has been modified from penetratin in part by inserting histidine residues in strategic spots in the peptide in order to add increased endosomolytic activity to the parent CPP. EB1 sequences include but are not limited to the following: LIRLWSHLIHIWFQNRRLKWKKK (SEQ ID NO:3643) Penetratin or EB1 can be used as a fusion peptide with enhanced CPP activity where the fusion partner is selected from peptides derived from the following group: (a) hemagglutinin esterase fusion protein (HEF) from influenza C virus; (b) HA2 and its analogs, see below and as an example of such a fusion peptide the following sequence: GLFGAIAGFIENGWEGMIDGRQIKIWFQNRRMKWKK (SEQ ID NO: 3644); (c) transmembrane glycoproteins from filovirus, rabies virus, see below, vesicular stomatitis virus or Semliki Forest virus; (d) fusion polypeptide of sendai virus, FFGAVIGTIALGVATA SEQ ID NO: 3645) human respiratory syncytial virus, FLGFLLGVGSAIASGV (SEQ ID NO: 3646), HIV gp41, GVFVLGFLGFLATAGS (SEQ ID NO: 3647), ebola GP2, GAAIGLAWIPYFGPAA, (SEQ ID NO: 3648) See WO 2008/022046), measles virus, Newcastle disease virus, visna virus, murine leukemia virus, human T-cell leukemia virus, simian immunodeficiency virus; or (e) M2 protein of influenza A virus.
(4) VP22—See the following references. (Suzuki et al., J Mol Cell Cardiology 36: 603, 2004; Hudecz et al., Med Res Rev 25: 679, 2005; Meade et al., Adv Drug Delivery Rev 59: 134, 2007; Meade and Dowdy Adv Drug Delivery Rev 60: 530, 2008; Jones et al., Br J Pharmacol 145: 1093, 2005; Xiong et al., BMC Neuroscience 8: 50, 2007; Lemken et al., Mol Ther 15: 310, 2007; Bamdad and Bell, Iran Biomed J 11: 53, 2007; Greco et al., Gene Ther 12: 974, 2005; Aints et al., J Gene Med 1: 275, 1999; U.S. Pat. No. 7,329,638, US 2005/0042753, US 2007/0105775, WO 2007/095152, WO 2008/008476, WO 2006/029078, WO 2006/0222657, WO2008/022046, WO 2006/053683, WO 2004/048545)—VR22 sequences include for example: DAATATRGRSAASRPTERPRAPARSASRPRRPVD (or E) (SEQ ID NO: 3649). In addition to being a potent CPP suitable for use with a wide variety of tissue and cell types, VP22 has the added ability to shuttle the NABT to secondary cells after having delivered it to an initial set of cells. VP22 can be used as a fusion peptide with enhanced CPP activity where the fusion partner is selected from peptides derived from the following group: (a) HEF from influenza C virus; (b) HA2 and its analogs; (c) transmembrane glycoproteins from filovirus; rabies virus, vesicular stomatitis virus or Semliki Forest virus; (d) fusion polypeptide of sendai virus, human respiratory syncytial virus, measles virus, Newcastle disease virus, visna virus, murine leukemia virus, human T-cell leukemia virus, simian immunodeficiency virus; or (e) M2 protein of influenza A virus.

VP22 has been shown to facilitate penetration of the blood brain barrier. See Kretz et al., Mol Ther 7: 659, (2003). VP22 can also be employed to deliver NABTs to heart tissue. See Suzuki et al., J Mol Cell Cardiology 36: 603, 2004. Xiong et al., Hum Gene Ther 18: 490, 2007 report that VP22 peptides also have utility for targeting skeletal muscle. Kretz et al., Mol Ther 7: 659, 2003 have described the use of VP22 peptides for facilitating delivery to the retina.

(5) Model amphipathic peptide (MAP)—See the following references. (Hudecz et al., Med Res Rev 25: 679, 2005; Meade et al., Adv Drug Delivery Rev 59: 134, 2007; Meade and Dowdy Adv Drug Delivery Rev 60: 530, 2008; Jones et al., Br J Pharmacol 145: 1093, 2005; Drin et al., AAPS PharmSci 4: 1, 2002, WO2008/022046, WO 2004/048545, WO 2008/093982)—MAP has broad application as a CPP and its peptide sequences include, but are not limited to, KLAKLLALKALKAALKLA (SEQ ID NO: 3650) and KLALKLALKALKAALKLA (SEQ ID NO: 3651).
(6) Pep-1—See the following references. (Morris et al., Nature Biotech 19: 1173, 2001; Kim et al., J Biochem Mol Biol 39: 642, 2006; Choi et al., Mol Cells 20: 401, 2005; An et al., Mol Cells 25: 55, 2008; Munoz-Morris et al., Biochem Biophys Res Commun 355: 877, 2007; Choi et al., Free Radic Biol Med 41: 1058, 2006; Cho et al., Neurochem Int 52: 659, 2008; An et al., FEBS J 275: 1296, 2008; Lee et al., BMB Rep 41: 408, 2008; Yune et al., Free Radic Biol Med published online ahead of print Jul. 27, 2008; Eum et al., Free Radic Biol Med 37: 1656, 2004; Weller et al., Biochem 44: 15799, 2005; Choi et al., FEBS Lett 580: 6755, 2006; Gros et al., Biochim Biophys Acta 1753: 384, 2006; US 2003/0119725, U.S. Pat. No. 6,841,535, US 2007/0105775, WO 2008/093982)—Pep-1 sequences include, but are not limited to, KETWWETWWTEWSQPKKKRKV (SEQ ID NO: 3652). Pep-1 is a CPP that can be operably linked to nanoparticles capable of delivery of NABTs to the cytoplasm of cells.

In addition to numerous other tissues and cell types, Pep-1 can be successfully used as a CPP for the delivery of NABTs and other large charged molecules to intracellular compartments of brain and spinal cord and cells. Such uses include the NABT treatment of various neurological disorders including but not limited to the following: ischemia-reperfusion injury (including stroke), spinal cord injury amyotrophic lateral sclerosis and Parkinson's Disease.

(7) Pep-1 Related Peptides—See the following US patent applications and issued patent. (US 2003/0119725, U.S. Pat. No. 6,841,535, US 2007/0105775)—Pep-1 belongs to a series of related CPPs that are effective carriers or carrier components for the delivery of potent NABTs into intracellular compartments. Pep-2 has the sequence KETWFETWFTEWSQPKKKRKV (SEQ ID NO: 3653). Two amino acid sequence patterns have been observed in closely related peptides with CPP activity. In these peptides, the term Xaa refers to a position in the sequence where either any amino acid or no amino acid is acceptable. The sequence pattern that includes Pep-1 is the following: KXaaXaaWWETWWXaaXaaXaaSQPKKXaaRKXaa (SEQ ID NO: 3654). Additional peptides in this family include the following sequences: KETWWETWWTEWSQPKKRKV (SEQ ID NO: 3655), KETWWETWWTEASQPKKRKV (SEQ ID NO: 3656), KETWWETWWETWSQPKKKRKV (SEQ ID NO: 3657), KETWWETWTWSQPKKKRKV (SEQ ID NO: 3658) and KWWETWWETWSQPKKKRKV (SEQ ID NO: 3659). The closely related pattern is as follows: KETWWETWWXaaXaaWSQPKKKRKV (SEQ ID NO: 3660).
(8) Fusion sequence-based protein (FBP)—See the following references. (Hudecz et al., Med Res Rev 25: 679, 2005; Drin et al., AAPS PharmSci 4: 1, 2002; WO 2004/048545)—FBP peptide sequences include but are not limited to GALFLGWLGAAGSTM (SEQ ID NO: 3661) and GALFLGWLGAAGSTMGAWSQPKKKRKV (SEQ ID NO: 3662) where the second sequence ends with a nuclear localization sequence from SV40 T antigen.
(9) bPrPp—See Hudecz et al., Med Res Rev 25: 679, 2005; Magzoub et al., Biochim Biophys Acta 1716: 126, 2005; Magzoub et al., Biochem 44: 14890, 2005; Magzoub et al., Biochem Biophys Res Commun 348: 379, 2006; and Biverstahl et al., Biochem 43: 14940, 2004). bPrPp is a CPP based on peptides that are found in bovine prions and includes the following sequence: MVKSKIGSWILVLFVAMWSDVGLCKKRPKP (SEQ ID NO: 3663). This peptide has endosomolytic as well as CPP activity.
(10) PG-1 (peptide protegrin)—See Drin et al., AAPS PharmSci 4: 1, 2002 Adenot et al., Chemotherapy 53: 73, 2007; U.S. Pat. No. 7,399,727). —PG-1 is a CPP originally isolated from porcine leukocytes. Use of PG-1 peptides to deliver the NABTs of the invention enhances intracellular delivery thereof. Such PG-1 containing molecules are sometimes referred to as SynB vectors. These vectors typically employ protegrin based peptides of varying lengths, for example, SynB1 (RGGRLSYSRRRFSTSTGR; (SEQ ID NO: 3664) and SynB3 (RRLSYSRRRF; (SEQ ID NO:3665).

In addition to numerous other tissue and cell types, PG-1 and SynB vectors comprising CPPs based on Syn B family peptides can be used to increase transport of NABTs across the blood brain barrier.

(11) Transportan and analogues such as TP-7, TP-9 and TP-10—See the following references. (Soomets et al., Biochim Biophys Acta 1467: 165, 2000; Hudecz et al., Med Res Rev 25: 679, 2005; Fisher et al., Gene Ther 11: 1264, 2004; Rioux, Curr Opin Investig Drugs 2: 364, 2001; E1-Andaloussi et al., J Control Release 110: 189, 2005; Lindgren et al., Bioconjugate Chem 11: 619, 2000; Pooga et al., FASEB J 12: 67, 1998, WO2008/022046, WO 2006/053683, WO 2004/048545, WO 2008/093982)—Transportin is approximately 27 amino acids in length and contains approximately 12 functional amino acids from the neuropeptide galanin and approximately 14 amino acids from the mast cell degranulating peptide mastoparan, a CPP in its own right. Typically these peptides are connected by a lysine. Transportan sequences include but are not limited to the following: GWTLNSAGYLLGKINLKALAALAKKIL (SEQ ID NO: 3666). The TP-10 sequence is the shortest of the transportan group, TP-7, TP-9 and TP-10 and is as follows: AGYLLGKINLKALAALAKKIL (SEQ ID NO: 3667).
(12) Protamine and Protamine-fragment/SV40 peptides—See Benimetskaya et al., Bioconjugate Chem 13: 177, 2002; U.S. Pat. No. 5,792,645, U.S. Pat. No. 7,329,638, and US 2005/0042753. Protamine-fragment/SV40 peptides are bifunctional CPPs composed of a C-terminal protamine-fragment that contains a DNA binding domain and an N-terminal nuclear localization signal derived from SV40 large T-antigen. One variant is called s-protamine-NLS and has sequences that include but are not limited to, R6WGR6-PKKKRKV (SEQ ID NO: 3668) while another, l-protamine-NLS, has sequences that include R4SR6FGR-6VWR4-PKKKRKV(SEQ ID NO: 3669). In addition to being combined with peptides from SV40, protamine itself has the capacity to promote uptake of NABTs into intracellular compartments.
(13) Polyethylenimine (PEI)—See the following references. (Intra and Salem, J Controlled Release 130: 129, 2008; Ogris et al., J Biol Chem 276: 47550, 2001; Breunig et al., J Gene Med 7: 1287, 2005; Loftus et al., Neurosci 139: 1061, 2006; Wang et al., Mol Therapy 3: 658, 2001; Boeckle et al., J Control Release 112: 240, 2006; U.S. Pat. No. 5,792,645, US 2003/0027784, US 2004/0185564, US 2008/0207553, WO 9602655, WO 00/59548, WO 2006/041617, WO 2004/029213, WO 03/099225, WO 2007/0135372, WO 94/01448)—PEI comes in linear and branched forms as well as in a low molecular weight form (<50,000 Daltons). It is a CPP-mimetic that has a particular advantage over other CPPs in that it is not subject to proteolysis. In addition to iv and im routes of administration, NABTs associated with a PEI containing carrier can be administered by aerosol delivery via the respiratory tract. Conjugation of PEI to certain melittin analogs provides added endosomolytic activity and, therefore, enhanced NABT delivery to intracellular sites where NABTs can carry out their intended function. PEI, as for most if not all CPPs, can be incorporated into nanoparticles to further promote the efficiency of NABT delivery to intracellular compartments. The specific methods for such CPP incorporation depend on the type of nanoparticle and are discussed in the reference documents provided herein for each type of nanoparticle. PEI can also be used to facilitate delivery of a NABT to the brain following intranasal administration. Also see Bhattacharya et al., Pharmaceut Res 25: 605, 2007; Zhang et al., J Gene Med 4: 183, 2002; Boado et al., Biotechnol Bioeng 96: 381, 2007; Coloma et al., Pharm Res 17: 266, 2000; US 2008/0051564, WO 94/13325, WO 99/00150, WO 2004/050016).
(14) Insulin and insulin-like growth factor receptor ligands—See Basu and Wickstrom, Bioconjugate Chem 8: 481, 1997; Zhang et al., J Gene Med 4: 183, 2002; Boado et al., Biotechnol Bioeng 96: 381, 2007; Coloma et al., Pharm Res 17: 266, 2000; Soos et al., Biochem J 235: 199, 1986; US 2008/0051564, WO 99/00150, WO 2004/050016 and U.S. Pat. No. 7,388,079)—Human Insulin receptor (HIR) monoclonal antibodies (MAbs) are directed to the human insulin receptor. Other suitable ligands include but are not limited to insulin, IGF-1 and IGF-2 or functional fragments thereof. Examples of IGF-1 binding peptides that can be used for this purpose include but are not limited to JB3 (D-C-S-K-A-P-K-L-P-A-A-Y-C (SEQ ID NO: 3670) where D denotes the D stereoisomer of C and where all the other stereoisomers are L) and JB9 (G-G-G-G-G-C-S-K-C; SEQ ID NO: 3671). Amide bond linked antisense oligos can be inserted between the first and second Gs of JB9. When incorporated into a carrier, these ligands can be used to deliver NABTs into cells that express this receptor. Such cells include but are not limited to liver, adipose tissue, skeletal muscle, cardiac muscle, brain, kidney and pancreas.

Insulin and insulin-like growth factor receptor ligands as described U.S. Pat. No. 4,801,575, WO 99/00150, WO 2004/050016, WO 2008/022349, WO 2005/035550, WO 2007/044323) are useful in methods targeting the CNS for delivery of NABTs specific for desired CNS targets. HIR monoclonal antibodies (HIR MAbs) are able to both cross the blood brain barrier as well as brain cell membranes. When conjugated to an NABT or incorporated into a carrier, such molecules facilitate transport of NABTs across the blood brain barrier. Other suitable ligands include IGF-1 and IGF-2 molecules and functional fragments thereof.

(15) Poly-Lysine—See Zhu et al., Biotechnol Appl Biochem 39: 179, 2004; Parker et al., J Gene Med 7: 1545, 2005; Stewart et al., Mol Pharm 50: 1487, 1996; U.S. Pat. No. 5,547,932, U.S. Pat. No. 5,792,645, WO 2006/053683, WO 2004/029213, and WO 93/04701. Poly-lysine consisting of approximately 3-20 amino acids can be used (D and L lysine stereoisomers both work) as carriers or as part of more complex carriers to transport NABTs into intracellular compartments where they can express their intended therapeutic effects. The CPP activity of poly-lysine can also be enhanced by glycosylation.
(16) Histidine-Lysine Peptides—See the following references. (Leng et al., Drug News Perspect 20: 77, 2007; U.S. Pat. No. 7,070,807, U.S. Pat. No. 7,163,695, US 2008/0171025, WO 01/47496, WO 2004/048421, WO 2006/060182)—Histidine-Lysine Peptides useful for the practice of the present invention come in both linear and branched forms. They may also be conjugated to polyethylene glycol and vascular specific ligands where they are particularly useful for delivering NABTs to the intracellular compartments of cells in solid tumors.
(17) Poly-Arginine—See Meade et al., Adv Drug Delivery Rev 59: 134, 2007; Meade and Dowdy Adv Drug Delivery Rev 60: 530, 2008; Jones et al., Br J Pharmacol 145: 1093, 2005; WO 2007/095152, WO 2008/008476, WO 2006/029078, WO 2006/0222657, WO 2006/053683, and WO 2004/029213. Poly-Arginine consisting of approximately 3-20 amino acids can be used (D and L lysine stereoisomers both work) as a fusion peptide with enhanced CPP activity where the fusion partner is selected from peptides derived from the following group: (a) HEF from influenza C virus; (b) HA2 and its analogs; (c) transmembrane glycoproteins from filovirus, rabies virus, vesicular stomatitis virus or Semliki Forest virus; (d) fusion polypeptide of sendai virus, human respiratory syncytial virus, measles virus, Newcastle disease virus, visna virus, murine leukemia virus, human T-cell leukemia virus, simian immunodeficiency virus; or (e) M2 protein of influenza A virus.
(18) NL4-10K—This molecule is described in Zeng et al., J Gene Med 6: 1247, 2004 and US 2005/0,048,606. —The NL4-10K peptide is based on nerve growth factor and has the sequence CTTTHTFVKALTMDGKQAAWRFIRIDTACKKKKKKKKKK (SEQ ID NO: 3672) and is typically used in a hairpin configuration. It facilitates uptake of NABTs into cells and tissues that express the nerve growth factor receptor TrkA. Alternative peptides based on nerve growth factor suitable for this purpose include, the following: TTATDIKGKEVMV (SEQ ID NO: 3673), EVNINNSVF(SEQ ID NO: 3674), RGIDSKHWNSY (SEQ ID NO: 3675) and TTTHTFVKALTMDGKQAAWRFIRIDTA (SEQ ID NO: 3676). Cells expressing TrkA include but are not limited to hepatocellular carcinoma, prostate cancer, neuroblastoma, melanoma, pancreatic cancer as well as non-malignant lung, pancreas, smooth muscle and prostate. NL4-10K peptides are suitable for getting NABTs across the blood brain barrier and into brain cells. US 2005/0048606 also provides CPPs suitable for promoting NABT uptake into cells that express the TrkB and TrkC receptors.
(19) S4₁₃-PV—See Mario et al., Biochem J 390: 603, 2005 and Mano et al., Biochimica Biophysica Acta 1758: 336, 2006. —S4₁₃-PV is a CPP that has a pronounced capacity to transport substances such as NABTs into cells without passing through endosomes. An exemplary sequence is ALWKTLLKKVLKAPKKKRKVC (SEQ ID NO: 3677).
(20) Sweet Arrow Peptide (SAP)—Foerg et al., Biochem 44: 72, 2005 described the SAP. —An exemplary SAP sequence is VRLPPPVRLPPPVRLPPP (SEQ ID NO: 3678).
(21) Human Calcitonin Derived Peptide hCT(9-32)—See Foerg et al., Biochem 44: 72, 2005. —hCT(9-32) has the following sequence LGTYTQDFNKFHTFPQTAIGVGAP, (SEQ ID NO: 3679).
(22) ARF based CPPs—See WO 2008/063113. —ARF based CPPs are 15-26 amino acids long comprising at least amino acids 1-14 of a mature mammalian ARF protein or a scrambled or partially inverted sequence thereof, optionally linked to one or more members of the group consisting of a cell-homing peptide, a receptor ligand, a linker and a peptide spacer comprising a selective protease cleavage site coupled to an inactivating peptide. A scrambled or partially inverted sequence of ARF defines a sequence wherein the same amino acids in the ARF sequence are included but one or several amino acids are in different positions so that part of the sequence is inverted or the whole sequence is scrambled. ARF sequences suitable for this use include but are not limited to human p14ARF and murine p19ARF. Suitable peptides for this use include but are not limited to M918 (MVTVLFRRLRIRRACGPPRVRV; (SEQ ID NO: 3680), M917 (MVRRFLVTLRIRRACGPPRVRV; (SEQ ID NO: 3681) and M872 (FVTRGCPRRLVARLIRVMVPRR; (SEQ ID NO: 3682).
(23) Kaposi FGF signal sequences—See Hudecz et al., Med Res Rev 25: 679, 2005; WO 2008/022046, and WO 2008/093982. —Kaposi FGF signal sequences include but are not limited to: AAVALLPAVLLALLAP (SEQ ID NO: 3683) and AAVLLPVLLPVLLAAP (SEQ ID NO: 3684).
(24) Human beta3 integrin signal sequence—See WO 2008/022046. —Human beta3 integrin signal sequences include: VTVLALGALAGVGVG, (SEQ ID NO: 3685).
(25) gp41 fusion sequence—See WO 2008/022046, and WO 2006/053683.)—gp41 fusion sequences include: GALFLGWLGAAGSTMGA (SEQ ID NO: 3686) which can be used as a CPP or combined with other CPPs to increase their endosomolytic function.
(26) Caiman crocodylus Ig(v) light chain—See the following references (Drin et al., AAPS PharmSci 4: 1, 2002; WO 2008/022046, WO 2006/053683, and WO 2004/048545. —Caiman crocodylus Ig(v) light chain sequences include: MGLGLHLLVLAAALQ (SEQ ID NO: 3687) and MGLGLHLLVLAAALQGAWSQPKKKRKV (SEQ ID NO: 3688) where the second sequence ends with a nuclear localization sequence from SV40 T antigen.
(27) hCT-derived peptide—See WO 2008/022046. —hCT-derived peptide sequences include: LGTYTQDFNKFHTFPQTAIGVGAP (SEQ ID NO: 3689).
(28) Loligomer—See WO 2008/022046. —An exemplary loligomer has the following sequence: TPPKKKRKVEDPKKKK (SEQ ID NO: 3690).
(29) Anthrax toxin derivatives—See the following references. (Arora and Leppla, J Biol Chem 268: 3334, 1993; Arora and Leppla, Infect Immun 62: 4955, 1994; Bradley et al., Nature 414: 225, 2001; Kushner et al., Proc Natl Acad Sci USA 100: 6652, 2003; Ballard et al., Proc Natl Acad Sci USA 93: 12531, 1996; Zhang et al., Proc Natl Acad Sci USA 101: 16756, 2004; Blanke et al., Proc Natl Acad Sci USA 93: 8437, 1996; Melnyk and Collier, Proc Natl Acad Sci USA 103: 9802, 2006; Krantz et al., Science 309: 777, 2005; Liu et al., Cell Microbiol 9: 977, 2007; U.S. Pat. No. 5,677,274, US 2003/0202989, US 2005/0220807, WO 97/23236, WO 03/087129, WO 2006/091233, and WO 94/18332)—Receptors for anthrax toxin are broadly found on the surfaces of various cell types. Anthrax toxin protective antigen (PA) is the portion of the anthrax toxin that is normally responsible for delivering the toxin to the cytoplasm of cells. PA functions both as a CPP and as an endosomolytic agent, is nontoxic, and can be used to promote the delivery of NABTs to the cytoplasm of cells. While PA is suitable, engineered peptides based on those regions of the PA domains directly involved in CPP and endosomolysis, along with certain other anthrax toxin sequences which augment these functions are most preferred. Anthrax lethal factor and fragments thereof also can be used to deliver NABTs into the cytoplasm of cells. Suitable engineered peptides based on anthrax sequences include, but are not limited to, ligation of a portion of the lethal factor sequence that contains the PA binding site with a sequence called the entry motif as provided by WO 2006/091233. Such engineered peptides can optionally be attached to a nuclear localization sequence. NABTs linked to polycationic tracts, e.g., polylysine, polyarginine and/or polyhistidine can further potentiate delivery of NABTs into the cytoplasm of cells.
(30) Ligands for transferrin receptor—See the following references. (U.S. Pat. No. 4,801,575, U.S. Pat. No. 5,547,932, U.S. Pat. No. 5,792,645, WO 2004/020404, WO 2004/020405, WO 2004/020454, WO 2004/020588, WO 2005/121179, WO 2006/049983, WO 2006/096515, WO 2008/033395, WO 2008/072075, WO 2008/022349, WO 2005/035550, WO 2007/044323 and WO 91/04753)—Ligands for transferrin receptor can be used to transport NABTs into cells which express this receptor. Such ligands include but are not limited to transferrin based peptides but can include other molecules such as peptides based on melanocortin, an integrin or glucagon-like peptide 1.

Ligands for the transferrin receptor can therefore be operably linked to the NABTs of the invention to facilitate transport of the therapeutic across the blood brain barrier in disorders where delivery to the CNS is desirable.

(31) Ligands for transmembrane domain protein 30A—See WO 2007/036021—Ligands for transmembrane domain protein 30A can be used to transport NABTs into cells that express this protein such as brain endothelium and can also be used to advantage to transport NABT across the blood brain barrier. Such ligands include antibodies and antibody fragments that bind the TMEM30A antigen as well as any one of several peptide ligands set forth in WO 2007/036021.
(32) Ligands for asialoglycoprotein receptor—See the following references. (Li et al., Sci China C Life Sci 42: 435, 1999; Huang et al., Int J Pharm 360: 197, 2008; Wang et al., J Drug Target 16: 233, 2008; Khorev et al., Bioorg Med Chem 16: 5216, 2008; WO 93/04701)—Ligands for asialoglycoprotein receptor can be used to transport NABTs into cells that express them, such as liver cells.
(33) Actively Transported Nutrients—See U.S. Pat. No. 6,528,631. —Actively transported nutrients can be directly conjugated to NABTs or associated with more complex carrier structures for the purpose of transporting said NABT into intracellular compartments. Exemplary nutrients for this purpose include, but are not limited to, folic acid, vitamin B6, vitamin B12, and cholesterol.
(34) UTARVE—See the following references. (Smith et al., International J Oncology 17: 841, 2000; WO 99/07723, WO 00/46384)—UTARVE refers to a vector for the delivery of NABTs into the cytoplasm of cells where the vector comprises a CPP or a ligand for a cell surface receptor that is internalized with the receptor and an influenza virus hemagglutinin peptide with endosomolytic activity. The CPP or cell surface receptor ligand can include any of those described herein. In addition, the ligand can be adenovirus penton peptide, epidermal growth factor receptor or the GM1 ganglioside receptor for cholera toxin B subunit. In addition, the vector may also include a polylysylleucyl peptide to provide additional NABT attachment sites and/or a nuclear localization signal. Adenovirus penton base proteins contain a receptor binding site motif (RGD) for attachment to integrins. Integrins are ubiquitous cell receptors. As used herein adenovirus penton base protein refers to the entire adenovirus penton base protein or to fragments thereof that include at least amino acids 1-354 that contain the receptor binding motif. The particular adenovirus from which the adenovirus penton base protein is derived is not critical and examples of such adenoviruses include but are not limited to Ad2, Ad3 and Ad5. These sequences are well known in the art. The influenza hemaglutinin peptide with endosomolytic activity is described elsewhere herein. The polylysylleucyl peptide has the sequence (KL)m where the lysine residues interact with the NABT while the leucine residues decrease the potential steric hindrance resulting from adjacent lysine residues. The value of m is not critical but generally represents from 1 to 300 alternating residues and preferably from 3 to 100. Should nuclear localization be desirable, a nuclear localization sequence, such as those discussed above, or otherwise well known in the art, may be employed.
(35) Antimicrobial peptides and their analogs—See the following references. (Sandgren et al., J Biol Chem 279: 17951, 2004; US 2004/0132970; US 2002/0082195, US 2004/0072990, US 2006/0069022, US 2007/0037744, US 2007/0065908, US 2007/0149448, US 2006/0128614, WO 2005/040201, WO 2006/011792, WO 2006/067402, WO 2006/076742, WO 2007/076162, WO 2007/148078, WO 2008/022444, WO 2006/050611, WO 2008/0125359)—Numerous antimicrobial peptides are naturally occurring and are involved in innate immunity. These peptides are typically cationic and function as CPPs and therefore can be harnessed to assist in the delivery of NABTs. The receptors for antimicrobial peptides are the cell surface proteoglycans, a major source of cell surface polyanions. While they are cytotoxic to microbes, antimicrobial peptides typically are much less toxic to mammalian cells. One such peptide is LL-37 which has the following sequence: LLGDFFRKSKEKIGKEFKRIVQRIKDFLRNLVPRTES (SEQ ID NO: 3691). Other examples involve peptides based on the dermaseptin family of antimicrobial peptides found on the skin of frogs of the Phylloinedusinae genus. Such peptides include, for example: ALWKTLLKKVLKA (SEQ ID NO: 3692), ALWKTLLKKVLKAPKKKRKV, (SEQ ID NO: 3693), PKKKRKVALWKTLLKKVLKA, (SEQ ID NO: 3694) and RQARRNRRRALWKTLLKKVLKA, (SEQ ID NO: 3695). Other suitable antimicrobial peptides or their analogs with CPP activity include but are not limited to novispirins, MUC7-12, CRAMP, PR-39, cryptdin-4, HBD-2, dermcidin, cecropin P1, maganin-2, granulysin and FALL-39. Such antimicrobial peptides are being developed as antimicrobial agents but also can be employed to enhance NABT delivery into cells. Analogs of antimicrobial peptides include but are not limited to those with D amino acid substitutions for their L stereoisomer counterparts for the purpose of reducing protease attack.
(36) Screened products of peptide and MAb fragment display libraries—See the following references. (Thomas et al., Pharmaceutical Res 24: 1564, 2007; WO 01/15511, WO 03/068942, WO 2007/143711, WO 97/17613, WO 97/17614)—A series of CPPs and MAb fragments with the capacity to transport NABTs into a broad range of cell types in a manner that promotes their biological activity have been identified through a series of screening steps starting with peptide or MAb fragment libraries. Indeed, a series of antibody single chain variable fragments (scFvs) with the capacity to bind to endothelial cells have been developed. Such scFvs can be used to advantage to facilitate transport NABTs into the endothelium. It is clear from such work that a wide range of effective CPP for the purposes of the present invention are readily available. A series of scFvs with the capacity to bind to endothelial cells and to cause the transport NABTs across the blood brain barrier have been developed and are described in the references provided.
(37) Designer CPPs—See the following references. (Rhee and Davis J Biol Chem 281: 1233, 2006; Kim et al., Exp Cell Res 312: 1277, 2006; Kaihatsu et al., Biochem 43: 14340, 2004; Hudecz et al., Med Res Rev 25: 679, 2005; Adenot et al., Chemotherapy 53: 73, 2007; U.S. Pat. No. 5,547,932, U.S. Pat. No. 7,329,638, U.S. Pat. No. 7,101,844, U.S. Pat. No. 6,200,801, U.S. Pat. No. 5,972,901, US 2005/0154188, US 2006/0228407, US 2004/0152653, US 2005/0042753, US 2003/0119725, US 2005/0239687, US 2005/0106598, US 2007/0129305, U.S. Pat. No. 6,841,535, US 2008/0182973, US 2009/0029387, WO 2007/069090, WO 00/34308, WO 00/62067, WO 2007/095152, WO 2007/056153, WO 2008/022046, US 2008/0234183, WO 2005/007854, WO 2007/053512, WO 2008/093982, WO 03/106491, WO 2004/016274, WO 03/097671, WO 01/08708, WO 97/46100, WO 06126865)—A large number of CPPs have been rationally designed based on the following: (i) a substantial number of potent CPPs have been identified beginning with those of natural origin; and (ii) effective CPPs typically can function as a prototype for other CPPs that share a set of similar properties related to amino acid composition, sequence patters and size. Such CPPs have subsequently been screened for activity and particularly active CPPs identified and tested in various carrier arrangements of the types provided herein. In addition, Hallbrink et al., have studied a broad range of CPPs and have developed comprehensive rules that describe CPP structure and function. They then applied these rules to generate a large number of Designer CPPs as described in US 2008/0234183 which claims priority to WO 03/106491. Design features that can be individually or in some instances in combination with one or more other such features can be used to generate designer CPPs are provided below:
(a) The design parameters disclosed in US 2008/0234183 include a bulk property value Z_{Σ}, a term called Bulkₕₐ that reflects the number of non-hydrogen atoms (e.g. C, N, S and O) in the side chains of the amino acids and a term hdb standing for the number of accepting hydrogen bonds for the side chains of the amino acids. Some examples of these Designer CPPs include the peptide sequenced IVIAKLKA (SEQ ID NO: 3696) and IVIAKLKANLMCKTCRLAK (SEQ ID NO: 3697);
(b) Those that include the peptide sequence KVKKQ (SEQ ID NO:3698);
(c) Those that include the D-amino acid peptide sequence D(AAKK)₄ (SEQ ID NO: 3699);
(d) Those that include the sequence PFVYLI (SEQ ID NO: 3700) including but not limited to the sequence CSIPPEVKFNKPFVYLI (SEQ ID NO: 3701) that has been termed the C105Y peptide;
(e) polycations consisting of various combinations of amines, substituted amines, guanidinium, substituted guanidinium, histidyl or substituted histidyl and organized into one of 60 different patters where a specific patterns repeats one to about 20 times (WO 2005/007854). These polycations can be directly attached to an NABT, attached to an NABT through a linker or indirectly associated through pRNA, nanoparticles, nanoparticles based on dendrimers, nanolattices, nanovesicles or micelles;
(f) An arginine-rich peptide of 8-16 subunits selected from X subunits, Y subunits and optional Z subunits including at least six X subunits, at least two Y subunits and at most three Z subunits where >50% of said subunits are X subunits and where (i) each X subunit independently represents arginine or an arginine analog said analog being a cationic alpha-amino acid comprising a side chain of the structure R¹N═C(NH₂)R² where R¹ is H or R; R² is R NH₂, NHR or NR₂ where R is lower alkyl or lower alkenyl and may further include oxygen or nitrogen; R¹ and R² may together from a ring; and the side chain is linked to said amino acid via R¹ or R²; (ii) each Y subunit independently represents a neutral amino acid —C(O)—(CHR)n-NH— where either n is 2 to 7 and each R is independently H or methyl or n is 1 and R is a neutral side chain selected from substituted or unsubstituted alkyl, alkenyl, alkynyl, aryl and aralkyl wherein said neutral side chain selected from substituted alkyl, alkenyl and alkynyl, includes at most one heteroatom for every four carbon atoms; and (iii) each Z subunit independently represents an amino acid selected from alanine, asparagine, cysteine, glutamine, glycine, histidine, lysine, methionine, serine and threonine.
(g) Sequences with the one of the following patterns were the term Xaa denotes either any amino acid or a position where an amino acid is not necessary with the noted preferred exceptions: XaaXaaXaaKKRRXaaXaaXaaXaaXaaXaaTWXaaETWWXaaXaaXaa (SEQ ID NO: 3702) (preferably at least one of the positions eight through thirteen is P, Q or G), YGFKKRRXaaXaaQXaaXaaXaaTWXaaETWWTE (SEQ ID NO: 3703) (preferably Xaa of position 16 is not omitted and preferably is an aromatic hydrophobic amino acid and is most preferably W) and YGFKKXRRPWTWWETWWTEX (SEQ ID NO: 3704) (preferably Xaa in position six is a hydrophobic amino acid, more preferably an aromatic hydrophobic amino acid and that the Xaa in position twenty is preferably omitted.
(h) A CPP comprising an amino acid sequence according to the general formula (X₁X₂B₁B₂X₃B₃X₄)n (SEQ ID NO: 3800) wherein X₁-X₄ are independently any hydrophobic amino acid; where in B₁, B₂ and B₃ are independently any basic amino acid; and wherein n is between 1 and 10.
(i) A CPP comprising an amino acid sequence according to either the general formula Q₁-X¹-(X²)₂-(X³)₂-X²-X⁴-X³-X⁴-X²-X⁴-X³-(X²)₂-Q₂ (SEQ ID NO: 3705) or Q₁-(X²)₂-X³-X⁴-X²-X⁴-X³-X⁴-X²-(X³)₂-(X²)₂-X¹-Q₂ (SEQ ID NO: 3706) where in one of Q₁ and Q₂ is H and the other of Q₁ and Q₂ is a covalent attachment to a linking moiety further attached to an NABT or to a carrier complex associated with an NABT; each X¹ is, independently, a naturally occurring or non-naturally occurring amino acid; each X², is independently, a D or L amino acid selected from lysine, histidine, homolysine, diaminobutyric acid, arginine, ornithine or homoarginine; each X³ is, independently, a D or L amino acid selected from alanine, valine, leucine, isoleucine, phenylalanine, tyrosine, tryptophan, cysteine, or methionine; and each X⁴ is, independently, a D or L amino acid selected from lysine, histidine, homolysine, diaminobutyric acid, arginine, ornithine, homoarginine, alanine, valine, leucine, isoleucine, phenylalanine, tyrosine, tryptophan, cysteine, methionine, glycine, serine, threonine, aspartate, glutamate, asparagine or glutamine.
(j) Those based on Syn B family peptides and generated using a computational model of cellular uptake followed by demonstrated ability to transport large charge molecules into intracellular compartments.
(k) CPPs have been designed that preferentially deliver NABTs to the cytoplasm of cells rather than to the nucleus. The CPP sequences useful for this purpose include but are not limited to the following sequence A-X₁-X₂-B-X₃-X₄-X₅-X₆-X₇-X₈ (SEQ ID NO: 3801) wherein A is an amino acid exhibiting relatively high freedom at the Φ and ω rotations of a peptide unit such as G or A, B is a basic amino acid and at least 3 residues of X₁-X₂-B-X₃-X₄-X₅-X₆-X₇-X₈ are R or K. CPP sequences useful for this purpose also include but are not limited to the following related sequences: YGRRARRRARR (SEQ ID NO: 3707), YGRRARRRARR (SEQ ID NO: 3708) and YGRRRRRRRRR (SEQ ID NO: 3709).

For example, designer ligands and CPPs have been described in the following references. See Costantino et al., J Controlled Release 108: 84, (2005), WO 2006/061101; WO 2007/143711 and WO 2005/035550. Exemplary ligands include those with one of the following sequences: HAIYPRH (SEQ ID NO: 3710) or THRPPMWSPVWP (SEQ ID NO: 3711). A designer CPP with the sequence H₂N-G-F-D-T-G-F-L-S-CONH₂ (SEQ ID NO: 3712), where D denotes the D stereoisomer of T and where all the other stereoisomers are L, that can be incorporated into nanoparticles suitable for transporting NABTs across the blood brain barrier. A designer CPP with the sequence H₂N-GF (specifically Phe-D) TGFLS-CONH₂ (SEQ ID NO: 3713) is well suited to carry NABTs into the cytoplasm of endothelial cells.

(38) Designer polycations that are not peptides—See U.S. Pat. No. 6,583,301; WO 99/02191. Designer polycations that are not peptides have been produced and shown to transport large charged molecules into intracellular compartments. These include but are not limited to structures that contain bipolar lipids with cationic heads, a hydrophobic backbone and a hydrophilic tail with a detailed structure as described in U.S. Pat. No. 6,583,301.
(39) Rabies virus glycoprotein (RVG) peptide—(U.S. Pat. No. 7,329,638, US 2005/0042753, WO 2008/054544)—The RVG peptide has sequences that include but are not limited to YTIWMPENPRPGTPCDIFTNSRGKRASNG (SEQ ID NO: 3714). When this peptide or a derivative or variant of it is used in a carrier for an NABT, it facilitates transport of the carrier/NABT complex across the blood brain barrier and into brain cells. In some embodiments the RVG peptide functions as a targeting agent and is conjugated to a carrier particle and an agent termed an effector agent (as defined by WO 2008/054544) that is associated with the carrier particle. In one embodiment said effector agent is a NABT. RVG may be used as the sole targeting agent or be used in combination with other targeting agents that include but are not limited to insulin, transferrin, insulin like growth factor, leptin, low density lipoprotein and fragments or peptidomimetics thereof. In some embodiments, the carrier particle is a lysosomal or polymeric nanoparticle, for example a liposome, polyarginine, protamine or a cyclodextrin-based nanoparticle. In alternative embodiments, the carrier particle is a CPP such as 11dR, 9dR, 7dR, 5dR or TAT or fragments thereof. 11dR, 9dR, 7dR and 5dR are polymeric arginine residues of varying length in these cases 11, 9, 7 and 5 arginines respectively.
(40) Ligands for leptin receptor—(WO 2008/022349, WO 2005/035550, WO 2007/044323)—Ligands for leptin receptor can be used to transport NABTs across the blood brain barrier.
(41) Ligands for lipoprotein receptor—(U.S. Pat. No. 5,547,932, WO 2008/022349, WO 2007/044323)—Ligands for lipoprotein receptor can be used to transport NABTs across the blood brain barrier.
(42) Hemagglutinating virus of Japan (HVJ) envelope. See the following references. Zhang et al., Biochem Biophys Res Commun 373: 345, 2008; Yamada et al., Am J Physiol 271: R1212, 1996; Bai et al., Ann Thorac Surg 66: 814, 1998; Ogata et al., Curr Eye Res 18: 261, 1999; Matsuo et al., J Drug Target 8: 207, 2000; Tomita et al., J Gene Med 4: 527, 2002; Okano et al., Gene Ther 10: 1381, 2003; Parveen et al., Virology 314: 74, 2003; Ferrari et al., Gene Ther 11: 1659, 2004; Sasaki et al., Gene Ther 12: 203, 2005; Griesenbach et al., Biomaterials 29: 1533, 2008; Kaneda et al., Mol Ther 6: 219, 2002; Kaneda et al., Expert Opin Drug Deliv 5: 221, 2008; Mima et al., J Gene Med 7: 888, 2005; Shimbo et al., Biochem Biophys Res Commun 364: 423, 2007; Kaneda et al., Adv Genet. 53: 307, 2005; Shimamura et al., Biochem Biophys Res Commun 300: 464, 2003; Morishita et al., Biochem. Biophys Res Commun 334: 1121, 2005; Kotani et al., Curr Gene Ther 4: 183, 2004; Hagihara et al., Gene Ther 7: 759, 2000; Ohmori et al., Eur J Cardio-thoracic Surg 27: 768, 2005; Tsujie et al., Kidney Inter 59: 1390, 2001; Yonemitsu et al., Gene Ther 4: 631, 1997; U.S. Pat. No. 6,913,923, US 2003/0013195, US 2004/0219674, US 2005/0239188, US 2006/0002894, WO 95/30330. Tissues where improved NABT uptake can be achieved by HVJ containing delivery systems include but are not limited to CNS, cardiovascular, uterus, liver, spleen, periodontal, skin, lung, retina, kidney, lymphoid tissues, embryonic stem cells and various solid tumors. In addition, carriers based on the HVJ envelope can be used to transfer NABTs across the blood brain barrier. Delivery has been via numerous routes including but not limited to topical, iv, intranasal, direct tissue injections including injection into amniotic fluid. This delivery system is particularly versatile and optionally includes nanoparticles and liposomes.
(43) Heart homing peptides are described in WO 00/75174 and include: GGGVFWQ (SEQ ID NO: 3715), HGRVRPH (SEQ ID NO: 3716), VVLVTSS (SEQ ID NO: 3717), CLHRGNSC (SEQ ID NO: 3718) and CRSWNKADNRSC (SEQ ID NO: 3719). These peptides can be directly conjugated to NABTs or be incorporated into more complex carriers. Further, they can be conjugated to or indirectly associated with other CPPs provided herein. The CRSWNKADNRSC (SEQ ID NO: 3719) peptide is particularly well suited to targeting regions of ischemia-reperfusion injury in the heart such as occurs in the treatment of heart attacks when the blood supply is medically restored.
(44) Peptides that target the LOX-1 receptor as described in White et al., Hypertension 37: 449, 2001) are particularly suitable for targeting NABTs to the endothelium. These peptides were initially selected from peptide libraries and then further screened for CPP activity. Examples include but are not limited to the following peptides: LSIPPKA (SEQ ID NO: 3720), FQTPPQL (SEQ ID NO: 3721) and LTPATAI (SEQ ID NO: 3722). LOX-1 is up-regulated on dysfunctional endothelial cells such as those involved in hypertension, diabetes, inflammation, restenosis, septic shock, ischemia-reperfusion injury and atherosclerosis and thus such peptides are particularly well suited for concentrating NABTs into this subset of cells to treat these and related medical conditions;
(45) Peptide for ocular delivery (POD) is described in Johnson et al., Mol Ther 16: 107, 2008)—POD has the following sequence GGG(ARKKAAKA)₄ (SEQ ID NO: 3723) and is suitable for transporting NABTs into the retina.
(46) LFA-1 targeting moieties are described in U.S. Pat. No. 7,329,638, US patent application 2005/0042753, International application WO 2007/127219. Preferred targeting moieties are selected from the group consisting of an antibody or a functional fragment thereof having immuno specificity for LFA-2 or protamine or a functional fragment thereof such as a peptide with the sequence RSQSRSRYYRQRQRSRRRRRRS (SEQ ID NO: 3724). Cells susceptible to LAF-1 targeting of NABTs include leukocytes and nerve cells as well as a variety of cancer cell types including but not limited to breast, colon and pancreas.
(47) PH-50—is described in WO 03/082213 and can be cross-linked and milled to generate nanoparticles to deliver NABTs to cells such as phagocytes involved in inflammation such as but not limited to those involved in ischemia reperfusion injury, arthritis and in atherosclerotic plaques.
(48) HA2 peptides are described in Dopheide et al., J Gen Virol 50: 329, 1980; Wang and El-Deiry, Trends Biotech 22: 431, 2004, Pichon et al., Antisense Nucleic Acid Drug Dev 7: 335, 1997; Daniels et al., Cell 40: 431, 1985; Navarro-Quiroga et al., Brain Res Mol Brain Res 105: 86, 2002; Cho et al., Biotechnol Appl Biochem 32: 21, 2000; Bailey et al., Biochim Biophys Acta 1324: 232, 1997; Steinhauer et al., J Virol 69: 6643, 1995; Sugita et al., Biochem Biophys Res Comm 363: 107, 2007; U.S. Pat. No. 5,547,932, WO 00/46384, WO 99/07723, and WO2008/022046. HA2 peptides can be employed in the compositions and methods of the invention to enhance endosomolysis to facilitate increased levels of NABT delivery. Influenza virus hemagglutinin (HA) is a trimer of identical subunits each of which contains two polypeptide chains HA1 and HA2. Functional HA2 sequences include but are not limited to: GLFGAIAGFIENGWEG (SEQ ID NO: 3725), GLFGAIAGFIGN(or G)GWGGMI(or V)D (SEQ ID NO: 3726) or GDIMGEWGNEIFGAIAGFLG (SEQ ID NO: 3727). In some instances, HA2 has been fused to the TAT CPP as described briefly above, to produce the dTAT-HA2 peptide. Such sequences include: RRRQRRKKRGGDIMGEWGNEIFGAIAGFLG (SEQ ID NO: 3728). dTAT-HA2 can more effectively deliver a bioactive NABT than TAT in instances where endosomal/lysosomal sequestration of the NABT reduces activity significantly.
(49) Poly-histidine and histidine requiring peptides See the following references. (Leng et al., Drug News Perspect 20: 77, 2007; McKenzie et al., Bioconjug Chem 11: 901, 2000; Reed et al., Nucleic Acids Res 33: e86, 2005; Lee et al., J Control Release 90: 363, 2003; Lo and Wang, Biomaterials 29: 2408, 2008, and WO 2006/053683)—Poly-histidine is hydrophobic at physiological pH but ionized at endosomal pH resulting in destabilization of the endosomal membrane. Polyhistidine can be operably linked to various CPPs to promote endosomolysis following cellular uptake. In some manifestations histidine is conjugated to poly(2-hydroxyethyl aspartamide) to produce an endosomolytic molecule capable of promoting the release of NABTs from endosomes, lysosomes or phagosomes. In another manifestation, approximately 10 histidines (preferred range 3 to 20 His) are conjugated to the C-terminus of TAT. In yet another embodiment, the aforementioned molecule comprises two cysteine residues which are incorporated into the molecule with a preferred distribution being cysteine-5 histidines-TAT-5 histidines-cysteine. Other histidine requiring peptides suitable for this purpose include but are not limited to the following: CHKKKKKKHC (SEQ ID NO: 3729), CHHHHHHKKKHHHHHHC (SEQ ID NO: 3730) and HHHHHWYG (SEQ ID NO: 3731).
(50) Sendi F1—(WO 2008/022046)—has the following sequence: FFGAVIGTIALGVATA (SEQ ID NO: 3732) which can be incorporated into fusion CPPs to increase their endosomolytic activity.
(51) Respiratory Syncytial Virus F1—(WO 2008/022046)—has the following sequence: FLGFLLGVGSAIASGV (SEQ ID NO: 3733) and can be incorporated into fusion CPPs to increase their endosomolytic activity.
(52) HIV gp41—(WO 2008/022046, WO 2006/053683)—has the following sequence: GVFVLGFLGFLATAGS (SEQ ID NO: 3734) can be incorporated into fusion CPPs to increase their endosomolytic activity.
(53) Ebola GP2—(WO 2008/022046)—has the following sequence: GAAIGLAWIPYFGPAA (SEQ ID NO: 3735) and can be incorporated into fusion CPPs to increase their endosomolytic activity.
(54) pH Triggered Agents See the following references (Ogris et al., J Biol Chem 276: 47550, 2001; Meyer et al., J Gene Med 9: 797, 2007; Chen et al., Bioconjug Chem 17: 1057, 2006; Boeckle et al., J Control Release 112: 240, 2006; Schreier, Pharm Acta Helv 68: 145, 1994; Martin and Rice, AAPS J 9: E18, 2007; Plank et al., Adv Drug Delivery Rev 34: 21, 1998; Wagner, Adv Drug Deliv Rev 38: 279, 1999; Eliyahu et al., Biomaterials 27: 1646, 2006; Eliyahu et al., Gene Therapy 12: 494, 2005; Provoda et al., J Biol Chem 278: 35102, 2003; Choi and Lee, J Controlled Release 131: 70, 2008; Parente et al., Biochem 29: 8720, 1990; Wyman et al., Biochem 36: 3008, 1997; Rittner et al., Mol Therapy 5: 104, 2002; US 2007/0036865, US 2004/0198687, US 2005/0244504, US 2003/0199090, US 2008/0187998, US 2006/0084617, U.S. Pat. No. 7,374,778, WO 2004/090107, WO 96/00792, WO 03/093449, WO 2006/053683, WO 94/01448)—pH Triggering Agents are agents that respond to the acidic pH found in endosomes/lysosomes or phagosomes in a manner that causes them to become endosomolytic. Such agents include certain viral proteins listed elsewhere herein but also include other peptides and small molecules that can be incorporated into a larger carrier molecule in multiple copies to concentrate their effect on endosomes/lysosomes (endosomolytic polymer). Endosomolytic polymers can be conjugated directly to NABTs by stable or by means of pH labile bonds or incorporated into nanoparticles carriers. Maleamates suitable for use as pH triggering agents include, but are not limited to, carboxydimethylmaleic anhydride, carboxydimethylmaleic anhydride-thioester and carboxydimethylmaleic anhydride-polyethylene glycol. In a preferred embodiment, a multiplicity of such maleamates (e.g., disubstituted maleic anhydride derivatives) are reversibly linked to polyamine as an endosomolytic polymer. Alternative pH triggering agents include but are not limited to the following:
(a) poly(beta-amino ester) as well as salts, derivatives, co-polymers and blends thereof;
(b) oligo sulfonamides including those with sulfamethizole, sulfadimethoxine, sulfadiazine or sulfamerazine moieties. Such oligo sulfonamides can be used without a separate endosomolytic polymer;
(c) Spermine where said spermine may include a cholesterol and/or fatty acid that may be bonded directly to a secondary amine in the spermine and said spermine may be further linked to a carbohydrate such as dextran or arabinogalactan;
(d) Peptides based on certain bacterial pore forming proteins such as listeriolysin O where the damage caused to cellular membranes around neutral pH is not unacceptably toxic. Listeriolysin O also can be beneficially combined with low molecular weight PEI to promote delivery of NABTs.
(e) Peptides and conjugates based on melittin (also called mellitin) of GIGAVLKVLTTGLPALISWIKRKRQQ (SEQ ID NO: 3736). Certain melittin analogues are better suited to this purpose than native melittin. Melittin-PEI conjugates are particularly preferred and are well suited as pH triggering agents. Exemplary conjugates include those where the N-terminus of melittin is conjugated to PEI. Further, modification of the C-terminally linked melittin peptide by replacement of the two neutral Q residues with E residues can increase the membrane lytic activity of melittin-PEI conjugates at endosomal pH. A preferred peptide structure with CPP and endosomolytic activity is a dimethylmaleic acid-melittin-polylysine conjugate. Melittin has also been developed into a gene delivery peptide capable of condensing and cross-linking DNA. This involves addition of lysine residues to increase the positive charge and terminal cysteine residues to promote polymerization.
(f) Alternative endosomolytic polymers include but are not limited to polyesters, polyanhydrides, polyethers, polyamides, polyacrylates, polymethacrylates, polycarbamates, polycarbonates, polyureas, poly(beta-amino esters) polythioesters and poly(alkyl)acrylic acids.
(g) The endosomolytic/pH triggering agents include but are not limited to peptides that contain imidazole groups or peptides having a repeating glutamate, alanine, leucine, alanine structure such as the EALA peptide (SEQ ID NO: 3737) (also known as GALA; SEQ ID NO: 3738) with a sequence that includes but is not limited to WEAALAEALAEALAEHLAEALAEALEALAA (SEQ ID NO: 3739) as well as the following: KALA (SEQ ID NO: 3740) with a sequence that includes but is not limited to WEAKLAKALAKALAKHLAKALAKALKACEA (SEQ ID NO: 3741), EGLA (SEQ ID NO: 3742), JTS-1 with a sequence that includes but is not limited to GLFEALLELLESLWELLLEA (SEQ ID NO: 3743), gramicidin S, ppTG1 with a sequence that includes but is not limited to GLFKALLKLLKSLWKLLLKA (SEQ ID NO: 3744) and ppTG20 with a sequence that includes but is not limited to GLFRALLRLLRSLWRLLLRA (SEQ ID NO: 3745).
(h) Any polymer which is not hydrophobic at physiologic pH but which becomes hydrophobic at pH (5.0-6.5) can be useful to promote endosomolysis and increase delivery of the NABT described herein. Further examples include: (a) Polymers that contain multiple carboxylic acid groups; and (b) Random, block and graft copolymers that include acrylate groups and alkyl substituted acrylate groups where preferably the alkyl group is a 1-6 carbon straight, branched or cyclic alkane. Preferred monomers for use in polymeric materials include poly(ethylacrylic acid), poly(propylacrylic acid) and poly(butylacrylic acid). Copolymers of these monomers by themselves or including acrylic acid can be used. Alternatively, or in addition, the carrier composition can include ligands such as poly-lysine or chitosan that can be associated with the NABT.

The ability of the molecules described above to move NABTs across cell membranes may be further enhanced by combining them with certain lipophilic domains and then combining the product with a NABT as described, for example, in Koppelhus et al., Bioconjugate Chem 19: 1526, 2008 and WO 2008/043366. Such lipophilic domains that may be conjugated to the CPP or to the NABT include but are not limited to the following: (1) an alkyl, alkenyl or alkynyl chain comprising 5-20 carbon atoms with a linear arrangement or including at least one cycloalkyl or heterocycle; or (2) a fatty acid containing 4 to 20 carbon atoms.

In certain embodiments of the invention, CPP, linkers, nanoparticles, nanoparticles based on dendrimers, nanolattices, nanovesicles, nanoribbons, liposomes or micelles used to associate such peptides to NABTs may be employed in the therapeutically beneficial compositions described herein. Such liposome applications include the use of heat delivery systems to promote targeting of heat labile liposomes carrying NABTs to particular tissues. Such compositions are described in Najlah and D'Emanuele, Curr Opin Pharmacol 6: 522, 2006; Munoz-Morris et al., Biochem Biophys Res Commun 355: 877, 2007; Lim et al., Angew Chem Int Ed 46: 3475, 2007; Zhu et al., Biotechnol Appl Biochem 39: 179, 2004; Huang et al., Bioconjug Chem 18: 403, 2007; Kolhatkar et al., Bioconjug Chem 18: 2054, 2007; Najlah et al., Bioconjug Chem 18: 937, 2007; Desgates et al., Adv Drug Delivery Rev 60: 537, 2008; Meade et al., Adv Drug Delivery Rev 59: 134, 2007; Albarran et al., Protein Engineering, Design & Selection 18: 147, 2005; Hashida et al., Br J Cancer 90: 1252, 2004; Ho et al., Cancer Res 61: 474, 2001; U.S. Pat. No. 7,329,638, US 2005/0042753, US 2006/0159619, US 2007/0077230, WO 2008/106503, WO 2008/073856, WO 2008/070141, WO 2008/045486, WO 2008/042686, WO 2008/003329, WO 2008/026224, WO 2008/037463, WO 2008/039188, WO2007/056153, WO2008/022046, WO 2007/131286, WO 2007/048019, WO 2004/048545, WO 2008/033253, WO 2005/035550, WO 0610247, and WO 2007/133182.

In certain embodiments, CPP are not employed to enhance uptake of the NABT of the invention. Compositions suitable for this embodiment are provided in the following references: Najlah and D'Emanuele, Curr Opin Pharmacol 6: 522, 2006; Huang et al., Bioconjug Chem 18: 403, 2007; Kolhatkar et al., Bioconjug Chem 18: 2054, 2007; Najlah et al., Bioconjug Chem 18: 937, 2007; US 2005/0175682, US 2007/0042031, U.S. Pat. No. 6,410,328, US 2005/0064595, US 2006/0083780, US 2006/0240093, US 2006/0051405, US 2007/0042031, US 2006/0240554, US 2008/0020058, US 2008/0188675, US 2006/0159619, WO 2008/096321, WO 2008/091465, WO 2008/073856, WO 2008/070141, WO 2008/045486, WO 2008/042686, WO 2008/003329, WO 2008/026224, WO 2008/037463, WO 2007/131286, WO 2007/048019, WO 2004/048545 WO 2007/0135372, WO 2008/033253, WO 2007/086881, WO 2007/086883, and WO 2007/133182.

In certain embodiments, it is preferable to deliver NABTs topically (e.g., to skin (e.g., for the treatment of psoriasis), mucus membranes, rectum, lungs and bladder). The following references describe compositions and methods that facilitate topical NABT delivery. See US 2005/0096287, US 2005/0238606, US 2008/0114281, U.S. Pat. No. 7,374,778, US 2007/0105775, WO 99/60167, WO 2005/069736, and WO 2004/076674. Exemplary methods and compositions include: (1) instruments that deliver a charge by means of electrodes to the skin with the result that the stratum corneum in an area beneath the electrodes is ablated thereby generating at least one micro-channel, the NABTs being administered optionally being associated with any of the NABT carriers described herein; (2) the use of ultrasound to both cross the skin and to assist in getting carrier/NABT complexes into cells; and (3) use of a carrier including but not limited to emulsions, colloids, surfactants, microscopic vesicles, a fatty acid, liposomes and transfersomes. The methods and compositions just provided in (2) and (3) and where the NABT has phosphodiester and/or phosphorothioate linkages may be further abetted by the use of reversible Charge Neutralization Groups of the type described in WO 2008/008476.

Polyampholyte complexes can be used to promote NABT uptake following topical application or following intravascular, intramuscular, intraperitoneal administration or by direct injections into particular tissues. In a preferred embodiment the polyampholyte complexes contain pH-labile bonds such as those described in US 2004/0162235, and WO 2004/076674.

Additional agents, CPPs and endosomolytic agents may be directly linked to NABTs or to carriers non-covalently associated with NABTs to improve the intracellular bioavailability of the NABT. Such agents include but are not limited to the compositions, methods and uses described in the following: Kubo et al., Org Biomol Chem 3: 3257, 2005; U.S. Pat. No. 5,574,142, U.S. Pat. No. 6,172,208, U.S. Pat. No. 6,900,297, US 2008/0152661, US 2003/0148928, WO 01/15737, WO 2008/022309, WO 2006/031461, WO 02/094185, WO 03/069306, WO 93/07883, WO 94/13325, WO 92/22332, WO 94/01448.

In order to cross intact mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores.

Liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over some other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized into an endosome. Due to the acidic pH within the endosome, the liposomes are ruptured, releasing their contents into the cell cytoplasm (Wang et al., Biochem Biophys Res Commun, 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., J Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome® I (glyceryl dilaurate/cholesterol/polyoxyethylene-10-stearyl ether) and Novasome® II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Scid., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. N.Y. Acad. Sci., 1987, 507, 64) reported the ability of monosialoganglioside GM1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., who disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, that contains a PEG moiety. Ilium et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating NABTs in liposomes. WO 97/04787 to Love et al. discloses liposomes comprising antisense NABTs targeted to the raf gene.

Transfersomes are yet another type of liposomes, and are highly deformable lipid aggregates which are attractive candidates for drug delivery vehicles. Transfersomes may be described as lipid droplets that are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes, it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The pharmacology of conventional antisense oligos with a variety of backbone chemistries and without the use of carriers has been extensively studied in many species, including humans. The backbones include the following: phosphorothioate, phosphorothioate gapmers with 2′-0-methyl ends, morpholino, LNA and FANA. The pharmacokinetics of these compounds is similar and these agents behave in a similar manner to many other drugs that are used systemically. As a result, the basic pharmacologic principals that have been established over the years apply here as well. For example, see the standard textbooks: “Principles of Drug Action: the Basis of Pharmacology”, WB Pratt and P Taylor, (editors), 3^{rd} edition, 1990, Churchill Livingston, 1990; Principles of Pharmacology: The Pathophysiologic Basis of Drug Therapy, D E Golan, AH Tashjian, EJ Armstrong and AW Armstrong (editors) 2^{nd} edition, 2007, Lippincott Williams & Wilkins. References that summarize much of pharmacology of all types of NABTs includes but are not limited to the following: Encyclopedia of Pharmaceutical Technology,-6 Volume Set, J Swarbrick (Editor) 3rd edition, 2006, Informa HealthCare; Pharmaceutical Perspectives of Nucleic Acid-Based Therapy, R I Mahato and SW Kim (Editors) 1 edition, 2002, CRC press; Antisense Drug Technology: Principles, Strategies, and Applications, ST Crooke (Editor) 2nd edition, 2007, Pharmaceutical Aspects of Oligonucleotides, P Couvreur and C Malvy (Editors) 1st edition, 1999, CRC press; Therapeutic Oligonucleotides (RSC Biomolecular Sciences) (RSC Biomolecular Sciences) (Hardcover) by Jens Kurreck (Editor) Royal Society of Chemistry; 1 edition, 2008, CRC press; Clinical Trials of Genetic Therapy with Antisense DNA and DNA Vectors, E Wickstrom (Editor) 1st edition, 1998, CRC press.

For the purposes of this invention, conventional antisense oligos can be administered intravenously (i.v.), intraperitoneally (i.p.), subcutaneously (s.c.), topically, or intramuscularly (i.m.). Antisense NABTs can be delivered intrathecally or used in combination with agents that interrupt or permeate the blood-brain barrier in order to treat conditions involving the central nervous system.

In certain embodiments, (e.g., for the treatment of lung disorders, such as pulmonary fibrosis or asthma or to allow for self administration) it may desirable to deliver the NABT described herein in aerolsolized form. A pharmaceutical composition comprising at least one NABT can be administered as an aerosol formulation which contains the inhibitor in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages in drops or sprays. Nasal solutions are generally prepared to be similar to nasal secretions and are generally isotonic and slightly buffered to maintain a pH of about 5.5 to about 6.5, although pH values outside of this range can additionally be used. Antimicrobial agents or preservatives can also be included in the formulation.

An aerosol formulation used for inhalations and inhalants is designed so that the NABT is carried into the respiratory tree of the patient administered by the nasal or oral respiratory route. See (WO 01/82868; WO 01/82873; WO 01/82980; WO 02/05730; WO 02/05785. Inhalation solutions can be administered, for example, by a nebulizer. Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the NABT. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The NABT can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as a NABT in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the NABT and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the NABT. The surfactant can be nonionic, anionic or cationic. One example of an emulsion can include, for example, ethanol, surfactant, water and propellant. Another example of an emulsion can include, for example, vegetable oil, glyceryl monostearate and propane.

As for many drugs, dose schedules for treating patients with NABTs can be readily extrapolated from animal studies. The extracellular concentrations that must be generally achieved with highly active conventional antisense oligos is in the 10-100 nanomolar (nM) range. Higher levels, up to 1.5 micromolar, may be more appropriate for some applications as this can result in an increase in the speed and amount of e oligo into the tissue thereby increasing tissue residence times. These levels can readily be achieved in the plasma. In the case of conventional antisense oligos, 1-10 mg/kg/day is a range that will cover most systemic applications with an infusion rate in the range of 0.1-1.5 mg/kg/hr. Intravenous administrations can be continuous or be over a period of minutes depending on the particular oligo. The primary determinants of the duration of treatment are the following: (1) the half-life of the target; (2) the richness of the blood supply to the target organ(s); and (3) the nature of the medical objective.

For ex vivo applications, the concentration of the conventional antisense oligos to be used is readily calculated based on the volume of physiologic balanced-salt solution or other medium in which the tissue to be treated is being bathed. In the large majority of applications, the oligos can be assumed to be stable for the duration of the treatment. With fresh tissue, 10-1000 nM represents the concentration extremes needed for a conventional antisense oligo with a reasonably good to excellent activity. Two hundred nanomolar (200 nM) is a generally serviceable level for most applications. Incubation of the tissue with the NABT at 5% rather than atmospheric (ambient) oxygen levels may improve the results significantly.

The following examples are provided to illustrate certain embodiments of the invention. They are not intended to limit the invention in any way.

### Example 1

### NABTs with Cardiovascular Applications and Methods of Use Thereof for the Treatment of Cardiovascular Disease

### A. Treatment of Cardiac Hypertrophy, MI, and Heart Failure.

Cardiovascular disease in the United States is associated with increasing morbity and mortality and thus new therapeutic agents for the treatment of this disorder are highly desirable. Such diseases include atherosclerosis, atherosclerotic plaque rupture, aneurisms (and ruptures thereof), coronary artery disease, cardiac hypertrophy, restenosis, vascular calcification, vascular proliferative disease, myocardial infarction and related pathologies which include, apoptosis of cardiac muscle, heart wall rupture, and ischemia reperfusion injury.

While several different therapeutic approaches are currently available to manage cardiovascular disease, e.g., heart failure, the incidence, prevalence, and economic costs of the disease are steadily increasing. The overall prevalence of congestive heart failure (CHF) is 1 to 2% in middle-aged and older adults, reaches 2 to 3% in patients older than age 65 years, and is 5 to 10% in patients beyond the age of 75 years (Yamani et al. (1993) Mayo Clin. Proc. 68:1214-1218).

Survival of patients suffering from heart failure depends on the duration and severity of the disease, on gender, as well as on previously utilized therapeutic strategies. In the Framingham study, the overall 5-year survival rates were 25% in men and 38% in women (Ho et al., (1993) Circulation 88:107-115). In clinical trials with selected patients under state-of-the-art medical therapy, 1 year mortality ranged between 35% in patients with severe congestive heart failure (NYHA IV) in the Consensus trial (The Consensus Trial Study Group (1987) N Engl. J. Med. 316:1429-1435) to 9 and 12% in patients with moderate CHF (NYHA II-III) in the second Vasodilator Heart Failure Trial (Cohn et al. (1991) N. Engl. J. Med. 325:303-310) and the Studies of Left Ventricular Dysfunction (SOLVD) trial. Mechanisms of death included sudden death in about 40%, and other factors in 20% of the patients.

The NABTs of the invention can be employed to diminish or alleviate the pathological symptoms associated with cardiac cell death due to apoptosis of heart cells. Initially the NABTs of interest will be incubated with a cardiac cell and the ability of the NABT to modulate targeted gene function (e.g., reduction in production of target gene product, apoptosis, improved cardiac cell signaling, Ca++ transport, or morphology etc) will be assessed. For example, the H9C2 cardiac muscle cell line can be obtained from American Type Culture Collection (Manassas, Va., USA) at passage 14 and cultured in DMEM complete culture medium (DMEM/F12 supplemented with 10% fetal calf serum (FCS), 2 mM α-glutamine, 0.5 mg/l Fungizone and 50 mg/l gentamicin). This cell line is suitable for characterizing the inhibitory functions of the NABTs of the invention and for characterization of modified versions thereof. HL-1 cells, described by Clayton et al. (1998) PNAS 95:2979-2984, can be repeatedly passaged and yet maintain a cardiac-specific phenotype. These cells can also be used to further characterize the effects of the NABTs described herein.

It may be desirable to further test the NABTs of the invention in animal models of heart failure. The tables below from Hasenfuss (1998) (Cardiovascular Research 39:60-76) provide a variety of animal models that are suitable for use in this embodiment of the invention. Each of the animal models described is useful for testing a biochemical parameter modulated by the NABTs provided herein. The skilled person can readily select the appropriate animal model and assess the effects of the NABT for its ability to ameliorate the symptoms associated with heart disease.

Heart failure is a serious condition that results from various cardiovascular diseases. p53 plays a significant role in the development of heart failure. Cardiac angiogenesis directly related to the maintenance of cardiac function as well as the development of cardiac hypertrophy induced by pressure-overload, and upregulated p53 induced the transition from cardiac hypertrophy to heart failure through the suppression of hypoxia inducible factor-1(HIF-1), which regulates angiogenesis in the hypertrophied heart. In addition, p53 is known to promote apoptosis, and apoptosis is thought to be involved in heart failure. Thus, p53 is a key molecule which triggers the development of heart failure via multiple mechanisms.

It appears that expression of the apoptosis regulator p53 is governed, in part, by a molecule that in mice is termed murine double minute 2 (MDM2), or, in man, human double minute 2 (HDM2), an E3 enzyme that targets p53 for ubiquitination and proteasomal processing, and by the deubiquitinating enzyme, herpesvirus-associated ubiquitin-specific protease (HAUSP), which rescues p53 by removing ubiquitin chains from it. Birks et al. (Cardiovasc Res. 2008 Aug. 1; 79(3): 472-80) examined whether elevated expression of p53 was associated with dysregulation of ubiquitin-proteasome system (UPS) components and activation of downstream effectors of apoptosis in human dilated cardiomyopathy (DCM). In these studies, left ventricular myocardial samples were obtained from patients with DCM (n=12) or from non-failing (donor) hearts (n=17). Western blotting and immunohistochemistry revealed that DCM tissues contained elevated levels of p53 and its regulators HDM2, MDM2 or the homologs thereof found in other species, and HAUSP (all P<0.01) compared with non-failing hearts. DCM tissues also contained elevated levels of polyubiquitinated proteins and possessed enhanced 20S-proteasome chymotrypsin-like activities (P<0.04) as measured in vitro using a fluorogenic substrate. DCM tissues contained activated caspases −9 and −3 (P<0.001) and reduced expression of the caspase substrate PARP-1 (P<0.05). Western blotting and immunohistochemistry revealed that DCM tissues contained elevated expression levels of caspase-3-activated DNAse (CAD; P<0.001), which is a key effector of DNA fragmentation in apoptosis and also contained elevated expression of a potent inhibitor of CAD (ICAD-S; P<0.01). These investigators concluded that expression of p53 in human DCM is associated with dysregulation of UPS components, which are known to regulate p53 stability. Elevated p53 expression and caspase activation in DCM was not associated with activation of both CAD and its inhibitor, ICAD-S. These findings are consistent with the concept that apoptosis may be interrupted and therefore potentially reversible in human HF.

In view of the foregoing, it is clear that the NABTs directed to p53 provided in Table 8 and 23 should exhibit efficacy for the treatment of heart failure. Accordingly, in one embodiment of the invention, the effects of p53 directed NABTs and their effects on cardiac cell apoptosis can be determined.

Additional NABTs for this purpose include, but are not limited to those targeting BCL-X, (Bcl-2-like 1; BCL2L1; BCL2L: Bcl-xS), FAS/APO1, Pro-apoptotic form of gene product, DB-1, (ZNF161; VEZF1), ICE (CASP1; Caspase-1), NF-kappaB, (Includes 51 KD, 65 KD and A subunits as well as intron 15), p53, PKC alpha, SRF and VEGF. In certain applications it may be desirable to conjugate the NABT to the CPP heart homing peptides described above. Preferred and most preferred NABT chemistries are described elsewhere herein.

Recently, Feng et al. reported that during myocardial ischemia, cardiomyocytes can undergo apoptosis or compensatory hypertrophy (Coron Artery Dis. 2008 November; 19(7):527-34). Fas expression is upregulated in the myocardial ischemia and is coupled to both apoptosis and hypertrophy of cardiomyocytes. Some reports suggested that Fas might induce myocardial hypertrophy. Apoptosis of ischemic cardiomyocytes and Fas expression in the nonischemic cardiomyocytes occurs during the early stage of ischemic heart failure. Hypertrophic cardiomyocytes easily undergo apoptosis in response to ischemia, after which apoptotic cardiomyocytes are replaced by fibrous tissue. In the late stage of ischemic heart failure, hypertrophy, apoptosis, and fibrosis are thought to accelerate each other and might thus form a vicious circle that eventually results in heart failure. Based on these observations, it is clear that NABTs targeting Fas provide useful therapeutic agents for ameliorating the pathological effects associated with myocardial ischemia and hypertrophy. Accordingly, fas directed NABTs will be applied to cardiac cells and their effects on apoptosis assessed. Fas directed NABTs will also be administered to animal models of heart failure to further characterize these effects. As discussed above in relation to p53 targeted NABTs, certain modifications of the NABT will also be assessed. These include conjugation to heart homing peptides, alterations to the phosphodiester backbone to improve bioavailability and stability, inclusion of CPPs, as well as encapusulation in liposomes or nanoparticles as appropriate.

Caspase-1/interleukin-converting enzyme (ICE) is a cysteine protease traditionally considered to have importance as an inflammatory mediator. Syed et al. examined the consequences of increased myocardial expression of procaspase-1 on the normal and ischemically injured heart (Circ Res. 2005 May 27; 96(10): 1103-9). In unstressed mouse hearts with a 30-fold increase in procaspase-1 content, unprocessed procaspase-1 was well tolerated, without detectable pathology. Cardiomyocyte processing and activation of caspase-1 and caspase-3 occurred after administration of endotoxin or with transient myocardial ischemia. In post-ischemic hearts, procaspase-1 overexpression was associated with strikingly increased cardiac myocyte apoptosis in the peri- and noninfarct regions and with 50% larger myocardial infarctions. Tissue culture studies revealed that procaspase-1 processing/activation is stimulated by hypoxia, and that caspase-1 acts in synergy with hypoxia to stimulate caspase-3 mediated apoptosis without activating upstream caspases. These data demonstrate that the proapoptotic effects of caspase-1 can significantly impact the myocardial response to ischemia and suggest that conditions in which procaspase-1 in the heart is increased may predispose to apoptotic myocardial injury under conditions of physiological stress. In view of these findings, NABTs directed to caspase 1 (ICE in Table 8) provide efficacious agents for the treatment of myocardial ischemia. Cardiac cells will be contacted with NABTs directed to ICE and the effects on cardiac cell apoptosis will be assessed. As mentioned previously, additional cardiac specific biochemical parameters such as Ca++ signaling, contractility, beta-adrenergic signaling, and cellular morphology can also be assessed. As above, several modifications can be engineered into the NABTs directed to ICE to increase cardiac cell homing, in vivo bioavailability and stability. These modified NABTs can then be further characterized in animal models of heart failure and hypertrophy.

Cardiac hypertrophy and dilation are also mediated by neuroendocrine factors and/or mitogens as well as through internal stretch- and stress-sensitive signaling pathways, which in turn transduce alterations in cardiac gene expression through specific signaling pathways. The transcription factor family known as myocyte enhancer factor 2 (MEF2 or MADS) has been implicated as a signal-responsive mediator of the cardiac transcriptional program. For example, known hypertrophic signaling pathways that utilize calcineurin, calmodulin-dependent protein kinase, and MAPKs can each affect MEF2 activity. Xu et al. demonstrate that MEF2 transcription factors induced dilated cardiomyopathy and lengthening of myocytes (J. Biol. Chem. (2006) Apr. 7; 281(14):9152-62). Specifically, multiple transgenic mouse lines with cardiac-specific overexpression of MEF2A or MEF2C presented with cardiomyopathy at base line or were predisposed to more fulminant disease following pressure overload stimulation. The cardiomyopathic response associated with MEF2A and MEF2C was not further altered by activated calcineurin, suggesting that MEF2 (MADS/MEF-2 in Table 8) functions independently of calcineurin in this response. In cultured cardiomyocytes, MEF2A, MEF2C, and MEF2-VP16 (a constitutively active mutant of MEF-2) overexpression induced sarcomeric disorganization and focal elongation. Mechanistically, MEF2A and MEF2C each programmed similar profiles of altered gene expression in the heart that included extracellular matrix remodeling, ion handling, and metabolic genes. Indeed, adenoviral transfection of cultured cardiomyocytes with MEF2A or of myocytes from the hearts of MEF2A transgenic adult mice showed reduced transient outward K(+) currents, consistent with the alterations in gene expression observed in transgenic mice and partially suggesting a proximal mechanism underlying MEF2-dependent cardiomyopathy. Based on the foregoing, NABTs directed to MEF-2 should have efficacy for the treatment of cardiomyopathy. Cardiomyocytes will be cultured in the presence of MEF-2 NABTs and the effects cardiac cell morphology and function will be determined to optimize dosage. As above, modifications to the NABTs directed to MEF-2 can also be assessed in the appropriate animal model provided below. As mentioned above, the animal models of cardiovascular disease listed in the following tables provide ideal in vivo models for optimizing the therapeutic efficacy and dosage of NABTs administration for the treatment of cardiovascular disease.

Animal models of heart failure

| Species and | | |
|---|---|---|
| technique | Selected references | Comments |
| Rat | | |
| Coronary | Pfeffer et al. (1979); Kajstura et al. (1994); | Clinical characteristics similar |
| ligation | Zarain-Herzberg et al. (1996); Liu et al. (1997) | to human CHF; survival studies |
| Aortic banding | Feldman et al. (1993); Weinberg et al. (1994); | Studies of transition from |
| | Shunkert et al. (1994) | hypertrophy to failure; survival |
| | | studies |
| Salt-sensitive | Dahl et al. (1962); Inoko et al. (1994) | Studies of transition from |
| hypertension | | hypertrophy to failure |
| Spontaneous | Okamoto et al. (1963); Bing et al. (1991); Boluyt | Extracellular matrix changes; |
| hypertension | et al. (1994); Li et al. (1997) | apoptosis; studies of transition |
| | | from hypertrophy to failure |
| SH-HF/Mcc-facp | Chua et al. (1996); Holycross et al. (1997); | Altered NOS expression; |
| | Narayan et al. (1995); Gomez et al. (1997); | altered calcium triggered |
| | Khaour et al. (1997) | calcium release |
| Aorto-caval | Jannini et al. (1996); Liu et al. (1991) | Left ventricular hypertrophy; |
| fistula | | moderate LV dysfunction |
| Toxic | Fein et al. (1994); Teerlink et al. (1994); Capasso | Decreased myocardial |
| cardiomyopathy | et al. (1992); Wei et al. (1997) | performance; myocyte loss with |
| | | chronic ethanol application. |
| | | Cardiomyopathy following |
| | | catecholamine infusion or |
| | | associated with Diabetes |
| | | mellitus |
| Dog | | |
| Pacing | Whipple G. H, et al. (1961); Armstrong P. W, et al. | Studies of remodeling and |
| tachycardia | (1986); Wilson J. R, et al. (1987); Ohno M, et al. | neurohumoral activation; |
| | (1994); Kiuchi K, et al. (1994); Armstrong P W, et | studies on molecular |
| | al. (1996); Eaton G. M, et al. (1995); Travill C. M, | mechanism of subcellular |
| | et al. (1992); Redfield M. M, et al. (1993); Luchner | dysfunction; no hypertrophy |
| | A, et al. (1996); Wang J, et al. (1994); Wolff M. R, | |
| | et al. (1995); O'Leary E. L, et al. (1992); Spinale | |
| | F. G, et al. (1995); Liu Y, et al. (1995); Ishikawa Y, | |
| | et al. (1994); Pak P. H, et al. (1997); Nuss H. B, et | |
| | al. (1996). | |
| Coronary artery | Sabbah H. N, et al. (1991); Gengo P. J, et al. (1992); | Studies on progression of heart |
| ligation | Gupta R. C, et al. (1997); Sabbah H. N, et al. (1994); | failure; high mortality and |
| | McDonald K. M, et al. (1992). | arrhythmias |
| Direct-current | McDonald K. M, et al. (1992). | Studies of neurohumoral |
| shock | | mechanisms |
| Volume overload- | McCullagh W. H, et al. (1972); Kleaveland J. P, et | Studies of neurohumoral |
| aorto-caval | al. (1988); Dell'Italia L. J. (1995); Nagatsu M, et al. | mechanisms and therapeutic |
| fistula-mitral | (1994); Tsutsui H, et al. (1994). | interventions |
| regurgitation | | |
| Vena caval | Wei C. M, et al. (1997). | Low cardiac output failure |
| constriction | | |
| Toxic | Magovern J. A, et al. (1992). | Left ventricular dysfunction |
| cardiomyopathy | | |
| Genetic | Cory C. R, et al. (1994). | Spontaneous cardiomyopathy in |
| | | Doberman Pinscher dogs |
| Pig | | |
| Pacing | Spinale F. G, et al. (1992); Spinale F. G, et al. | Comparable with dog model for |
| tachycardia | (1990); Spinale F. G, et al. (1991); Spinale F. G, et | most aspects |
| | al. (1994). | |
| Coronary artery | Zhang J, et al. (1996). | Congestive heart failure; altered |
| ligation | | myocardial energetic |
| Rabbit | | |
| Volume and | Magid N.M, et al. (1994); Gilson N, et al. (1990); | Myocardial alterations similar |
| pressure | Ezzaher A, et al. (1991); Ezzaher A, et al. (1992); | to failing human myocardium |
| overload | Pogwizd S. M, et al. (1997). | |
| Pacing | Freeman G. L, et al. (1992); Masaki H, et al. | Myocardial alteration similar to |
| tachycardia | (1993); Masaki H, et al. (1994); Ryu K. H, et al. | failing human myocardium |
| | (1997); Eble D. M, et al. (1997), Colston J. T, et al. | |
| | (1994). | |
| Toxic | Dodd D. A, et al. (1993). | Studies of functional |
| cardiomyopathy | | consequences of altered |
| | | ryanodine receptors |
| Guinea pig | | |
| Aortic banding | Kiss E, et al. (1995); Malhotra A, et al. (1992); Siri | Myocardial function and |
| | F. M, et al. (1989). | alteration of calcium handling |
| | | similar to human heart failure |
| Syrian hamster | | |
| Genetic | Bajusz E. (1969); Forman R, et al. (1972). Jasmin | Hypertrophy and failure; |
| | G, et al. (1982); Rouleau J. L, et al. (1982); | alterations critically dependent |
| | Whitmer J. T, et al. (1988); Finkel M. S, et al. | on strain and age |
| | (1987); Wagner J. A, et al. (1986); Kuo T. H, et al. | |
| | (1992); Hatem S. N, Set al. (1994); Malhotra A, et | |
| | al. (1985); Okazaki Y, et al. (1996); Nigro V, et al. | |
| | (1997). | |
| Cat | | |
| Pulmonary artery | Tagawa H, et al. (1996); Kent R. L, et al. (1993). | Transition from compensated |
| constriction | | right ventricular hypertrophy to |
| | | failure |
| Turkey | | |
| Toxic | Genao A, et al. (1996). | Alteration of calcium handling |
| cardiomyopathy | | and myocardial energetic |
| Bovine | | |
| Genetic | Eschenhagen T, et al. (1995). | Similar to human heart failure |
| | | regarding changes in (β- |
| | | adrenergic system |
| Sheep | | |
| Pacing | Rademaker M. T, et al. (1997); Rademaker M. T, | Similar to dog and swine model |
| tachycardia | et al. (1996). | of pacing tachycardia |
| Aortic | Aoyagi T, et al. (1993). | Transition from compensated |
| constriction | | hypertrophy to left ventricular |
| | | dysfunction |

Pfeffer M. A, Pfeffer J. M, Fishbein M. C, et al. Myocardial infarct size and ventricular function in rats. Circ Res 44:503-512.
Kajstura J, Zhang X, Reiss K, et al. Myocyte cellular hyperplasia and myocyte cellular hypertrophy contribute to chronic ventricular remodeling in coronary artery narrowing-induced cardiomyopathy in rats. Circ Res (1994) 74:383-400.
Zarain-Herzberg A, Afzal N, Elimban V, Dhalla N. S. Decreased expression of cardiac sarcoplasmic reticulum Ca2+-pump ATPase in congestive heart failure due to myocardial infarction. Mol Cell Biochem (1996) 163, 164:285-290.
Liu Y. H, Yang X. P, Nass O, et al. Chronic heart failure induced by coronary artery ligation in Lewis inbred rats. Am J Physiol (1997) 272:H722-727.
Feldman A. M, Weinberg E. O, Ray P. E, Lorell B. H. Selective changes in cardiac gene expression during compensated hypertrophy and the transition to cardiac decompensation in rats with chronic aortic banding. Circ Res (1993) 73:184-192.
Weinberg E. O, Schoen F. J, George D, et al. Angiotensin-converting enzyme inhibition prolongs survival and modifies the transition to heart failure in rats with pressure overload hypertrophy due to ascending aortic stenosis. Circulation (1994) 90:1410-1422.
Schunkert H, Lorell B. H. Role of angiotensin II in the transition of left ventricular hypertrophy to cardiac failure. Heart Failure (1994) 10:142-149.
Okamoto K, Aoki K. Development of a strain of spontaneously hypertensive rats. Jpn Circ J (1963) 27:282-293.
Bing O. H, Brooks W. W, Conrad C. H, et al. Intracellular calcium transients in myocardium from spontaneously hypertensive rats during the transition to heart failure. Circ Res (1991) 68:1390-1400.
Boluyt M. O, O'Neill L, Meredith A. L, et al. Alterations in cardiac gene expression during the transition from stable hypertrophy to heart failure. Marked upregulation of genes encoding extracellular matrix components. Circ Res (1994) 75:23-32.
Li Z, Bing O. H, Long X, Robinson K. G, Lakatta E. G. Increased cardiomyocyte apoptosis during the transition to heart failure in the spontaneously hypertensive rat. Am J Physiol (1997) 272:H2313-H2319.
Chua S. C Jr., Chung W. K, Wu-Peng X. S. Phenotypes of mouse diabetes and rat fatty due to mutations in the OB (leptin) receptor. Science (1996) 271:994-996.
Holycross B. J, Summers B. M, Dunn R. B, McCune S. A. Plasma-renin activity in heart failure-prone SHHF/Mcc-facp rats. Am J Physiol (1997) 273:H228-H233.
Narayan P, McCune S. A, Robitaille P. M, Hohl C. M, Altschuld R. A. Mechanical alternans and the force-frequency relationship in failing rat hearts. J Mol Cell Cardiol (1995) 27:523-530.
Gomez A. M, Valdivia H. H, Cheng H, et al. Defective excitation-contraction coupling in experimental cardiac hypertrophy and heart failure. Science (1997) 276:800-806.
Khadour F. H, Kao R. H, Park S, et al. Age-dependent augmentation of cardiac endothelial NOS in a genetic rat model of heart failure. Am J Physiol (1997) 273:H1223-H1230.
Lompre A. M, Mercadier J. J, Wisnewsky C, et al. Species and age-dependent changes in the relative amounts of cardiac myosin isozymes in mammals. Dev Biol (1981) 84:286-290.
Jannini J. P, Spinale F. G. The identification of contributory mechanisms for the development and progression of congestive heart failure in animal models. J Heart Lung Transplant (1996) 15:1138-1150.
Liu Z, Hilbelink D. R, Crockett W. B, Gerdes A. M. Regional changes in hemodynamics and cardiac myocyte size in rats with aortocaval fistulas. Developing and established hypertrophy. Circ Res (1991) 69:52-58.
Fein F. S, Sonnenblick E. H. Diabetic cardiomyopathy. Cardiovasc Drugs Ther (1994) 8:65-73.
Teerlink J. R, Pfeffer J. M, Pfeffer M. A. Progressive ventricular remodeling in response to diffuse isoproterenol-induced myocardial necrosis in rats. Circ Res (1994) 75:105-113.
Capasso J. M, Li P, Guideri G, et al. Myocardial mechanical, biochemical and structural alterations induced by chronic ethanol ingestion in rats. Circ Res (1992) 71:346-356.
Wei C. M, Clavell A. L, Burnett J. C. Atrial and pulmonary endothelin mRNA is increased in a canine model of chronic low cardiac output. Am J Physiol (1997) 273:R838-844.
Whipple G. H, Sheffield L. T, Woodman E. G, Thoephilis C, Friedman S. Reversible congestive heart failure due to rapid stimulation of the normal heart. Proc New Eng Cardiovasc Soc (1961) 20:39-40.
Armstrong P. W, Stopps T. P, Ford S. E, de Bold A. J. Rapid ventricular pacing in the dog: pathophysiologic studies of heart failure. Circulation (1986) 74:1075-1084.
Wilson J. R, Douglas P, Hickey W. F, et al. Experimental congestive heart failure produced by rapid ventricular pacing in the dog: cardiac effects. Circulation (1987) 75:857-867.
Ohno M, Cheng C. P, Little W. C. Mechanism of altered patterns of left ventricular filling during the development of congestive heart failure. Circulation (1994) 89:2241-2250.
Kiuchi K, Shannon R. P, Sato N, et al. Factors involved in delaying the rise in peripheral resistance in developing heart failure. Am J Physiol (1994) 267:H211-H216.
Armstrong P W, Gordon W M. The development of and recovery from pacing-induced heart failure. In: Spinale F G, editor. Pathophysiology of tachycardia-induced heart failure. Armonk, N.Y.: Futura Publishing Company, 1996; 45-59.
Eaton G. M, Cody R. J, Nunziata E, Binkley P. F. Early left ventricular dysfunction elicits activation of sympathetic drive and attenuation of parasympathetic tone in the paced canine model of congestive heart failure. Circulation (1995) 92:555-561.
Travill C. M, Williams T. D, Pate P, et al. Haemodynamic and neurohumoral response in heart failure produced by rapid ventricular pacing. Cardiovasc Res (1992) 26:783-790.
Redfield M. M, Aarhus L. L, Wright R. S, Burnett J. C Jr. Cardiorenal and neurohumoral function in a canine model of early left ventricular dysfunction. Circulation (1993) 87:2016-2022.
Luchner A, Stevens T. L, Borgeson D. D, et al. Angiotensin II in the evolution of experimental heart failure. Hypertension (1996) 28:472-477.
Wang J, Seyedi N, Xu X. B, Wolin M. S, Hintze T. H. Defective endothelium-mediated control of coronary circulation in conscious dogs after heart failure. Am J Physiol (1994) 266:H670-H680.
Wolff M. R, Whitesell L. F, Moss R. L. Calcium sensitivity of isometric tension is increased in canine experimental heart failure. Circ Res (1995) 76:781-789.
O'Leary E. L, Colston J. T, Freeman G. L. Maintained length-dependent activation of skinned myocardial fibers in tachycardia heart failure. Circulation (1992) 86(suppl I):284.
Spinale F. G, Holzgrefe H. H, Mukherjee R, et al. Angiotensin-converting enzyme inhibition and the progression of congestive cardiomyopathy. Effects on left ventricular and myocyte structure and function. Circulation 92:562-578.
Liu Y, Cigola E, Cheng W, et al. Myocyte nuclear mitotic division and programmed myocyte cell death characterize the cardiac myopathy induced by rapid ventricular pacing in dogs. Lab Invest (1995) 73:771-787.
Ishikawa Y, Sorota S, Kiuchi K, et al. Downregulation of adenylylcyclase types V and VI mRNA levels in pacing-induced heart failure in dogs. J Clin Invest (1994) 93:2224-2229.
Pak P. H, Nuss H. B, Kaab S, et al. Repolarization abnormalities, arrhythmia and sudden death in canine tachycardia induced cardiomyopathy. J Am Coll Cardiol (1997) 30:576-584.
Nuss H. B, Johns D. C, Kaab S, et al. Reversal of potassium channel deficiency in cells from failing hearts by adenoviral gene tranfer: a prototype for gene therapy for disorders of cardiac excitability and contractility. Gene Ther (1996) 3:900-912.
Sabbah H. N, Stein P. D, Kono T, et al. A canine model of chronic heart failure produced by multiple sequential coronary microembolizations. Am J Physiol (1991) 260:H1379-H1384.
Gengo P. J, Sabbah H. N, Steffen R. P, et al. Myocardial beta adrenoceptor and voltage-sensitive calcium channel changes in a canine model of chronic heart failure. J Mol Cell Cardiol (1992) 24:1361-1369.
Gupta R. C, Shimoyama H, Tanimura M, et al. SR Ca²⁺-ATPase activity and expression in ventricular myocardium of dogs with heart failure. Am J Physiol (1997) 273:H12-H18.
Sabbah H. N, Shimoyama H, Kono T, et al. Effects of long-term monotherapy with enalapril, metoprolol, and digoxin on the progression of left ventricular dysfunction and dilation in dogs with reduced ejection fraction. Circulation (1994) 89:2852-2859.
McDonald K. M, Francis G. S, Carlyle P. F, et al. Hemodynamic, left ventricular structural and hormonal changes after discrete myocardial damage in the dog. J Am Coll Cardiol (1992) 19:460-467.
McCullagh W. H, Covell J. W, Ross J Jr. Left ventricular dilatation and diastolic compliance changes during chronic volume overloading. Circulation (1972) 45:943-951.
Kleaveland J. P, Kussmaul W. G, Vinciguerra T, Diters R, Carabello B. A. Volume overload hypertrophy in a closed-chest model of mitral regurgitation. Am J Physiol (1988) 254:H1034-H1041.
Dell'Italia L. J. The canine model of mitral regurgitation. Heart Failure (1995) 11:208-218.
Nagatsu M, Zile M. R, Tsutsui H, et al. Native beta-adrenergic support for left ventricular dysfunction in experimental mitral regurgitation normalizes indexes of pump and contractile function. Circulation (1994) 89:818-826.
Tsutsui H, Spinale F. G, Nagatsu M, et al. Effects of chronic beta-adrenergic blockade on the left ventricular and cardiocyte abnormalities of chronic canine mitral regurgitation. J Clin Invest (1994) 93:2639-2648.
Wei C. M, Clavell A. L, Burnett J. C. Atrial and pulmonary endothelin mRNA is increased in a canine model of chronic low cardiac output. Am J Physiol (1997) 273:R838-844.
Magovern J. A, Christlieb I. Y, Badylak S. F, Lantz G. C, Kao R. L. A model of left ventricular dysfunction caused by intracoronary adriamycin. Ann Thorac Surg (1992) 53:861-863.
Cory C. R, Shen H, O'Brien P. J. Compensatory asymmetry in down-regulation and inhibition of the myocardial Ca²⁺ cycle in congestive heart failure produced in dogs by idiopathic dilated cardiomyopathy and rapid ventricular pacing. J Mol Cell Cardiol (1994) 26:173-184.
Spinale F. G, Fulbright B. M, Mukherjee R, et al. Relation between ventricular and myocyte function with tachycardia-induced cardiomyopathy. Circ Res (1992) 71:174-187.
Spinale F. G, Hendrick D. A, Crawford F. A, et al. Chronic supraventricular tachycardia causes ventricular dysfunction and subendocardial injury in swine. Am J Phys (1990) 259:H218-H229.
Spinale F. G, Tomita M, Zellner J. L, et al. Collagen remodeling and changes in LV function during development and recovery from supraventricular tachycardia. Am J Physiol (1991) 261:H308-H318.
Spinale F. G, Tempel G. E, Mukherjee R, et al. Cellular and molecular alterations in the beta adrenergic system with cardiomyopathy induced by tachycardia. Cardiovasc Res (1994) 28:1243-1250.
Zhang J, Wilke N, Wang Y, et al. Functional and bioenergetic consequences of postinfarction left ventricular remodeling in a new porcine model. MRI and 31 P-MRS study. Circulation (1996) 94:1089-1100.
Magid N. M, Opio G, Wallerson D. C, Young M. S, Borer J. S. Heart failure due to chronic experimental aortic regurgitation. Am J Physiol (1994) 267:H556-H562.
Gilson N, el Houda Bouanani N, Corsin A, Crozatier B. Left ventricular function and beta-adrenoceptors in rabbit failing heart. Am J Physiol (1990) 258:H634-H641.
Ezzaher A, Bouanani N. E. H, Su J. B, Hittinger L, Crozatier B. Increased negative inotropic effect of calcium channel blockers in hypertrophied and failing rabbit hearts. J Pharmacol Exp Ther (1991) 257:466-471.
Ezzaher A, Boudanani N. E. H, Crozatier B. Force-frequency relations and response to ryanodine in failing rabbit hearts. Am J Physiol (1992) 263:H1710-H1715.
Pogwizd S. M, Qi M, Samarel A. M, Bers D. M. Upregulation of Na⁺/Ca²⁺-exchanger gene expression in an arrhythmogenic model of nonischemic cardiomyopathy in the rabbit. Circulation (1997) 96(Suppl I):8.
Freeman G. L, Colston J. T. Myocardial depression produced by sustained tachycardia in rabbits. Am J Physiol 262:H63-H67.
Masaki H, Imaizumi T, Ando S, et al. Production of chronic congestive heart failure by rapid ventricular pacing in the rabbit. Cardiovasc Res (1993) 27:828-831.
Masaki H, Imaizumi T, Harasawa Y, Takeshita A. Dynamic arterial baroreflex in rabbits with heart failure induced by rapid pacing. Am J Physiol (1994) 267:H92-H99.
Ryu K. H, Tanaka N, Dalton N, et al. Force-frequency relations in the failing rabbit heart and responses to adrenergic stimulation. J Card Fail (1997) 3:27-39.
Eble D. M, Walker J. D, Mukherjee R, Samarel A. M, Spinale F. G. Myosin heavy chain synthesis is increased in a rabbit model of heart failure. Am J Physiol (1997) 272:H969-H978.
Siri F. M, Nordin C, Factor S. M, Sonnenblick E, Aronson R. Compensatory hypertrophy and failure in gradual pressure-overloaded guinea pig heart. Am J Physiol (1989) 257:H1016-H1024.
Bajusz E. Hereditary cardiomyopathy: a new disease model. Am Heart J (1969) 7:686-696.
Forman R, Parmley W. W, Sonnenblick E. H. Myocardial contractility in relation to hypertrophy and failure in myopathic Syrian hamsters. J Mol Cell Cardiol (1972) 4:203-211.
Jasmin G, Proschek L. Hereditary polymyopathy and cardiomyopathy in the Syrian hamster. I. Progression of heart and skeletal muscle lesions in the UM-X7.1 line. Muscle Nerve (1982) 5:20-25.
Rouleau J. L, Chuck L. H, Hollosi G, et al. Verapamil preserves myocardial contractility in the hereditary cardiomyopathy of the Syrian hamster. Circ Res (1982) 50:405-412.
Whitmer J. T, Kumar P, Solaro R. J. Calcium transport properties of cardiac sarcoplasmic reticulum from cardiomyopathic Syrian hamsters (BIO 53.58 and 14.6): evidence for a quantitative defect in dilated myopathic hearts not evident in hypertrophic hearts. Circ Res (1988) 62:81-85.
Finkel M. S, Marks E. S, Patterson R. E, et al. Correlation of changes in cardiac calcium channels with hemodynamics in Syrian hamster cardiomyopathy and heart failure. Life Sci (1987) 41:153-159.
Wagner J. A, Reynolds I. J, Weisman H. F, et al. Calcium antagonist receptors in cardiomyopathic hamster: selective increases in heart, muscle, brain. Science (1986) 232:515-518.
Kuo T. H, Tsang W, Wang K. K, Carlock L. Simultaneous reduction of the sarcolemmal and SR calcium ATPase activities and gene expression in cardiomyopathic hamster. Biochim Biophys Acta (1992) 1138:343-349.
Hatem S. N, Sham J. S, Morad M. Enhanced Na⁺/Ca²⁺ exchange activity in cardiomyopathic Syrian hamster. Circ Res (1994) 74:253-261.
Malhotra A, Karell M, Scheuer J. Multiple cardiac contractile protein abnormalities in myopathic Syrian hamsters (BIO 53: 58). J Mol Cell Cardiol (1985) 17:95-107.
Okazaki Y, Okuizumi H, Osumi T, et al. A genetic linkage map of the Syrian hamster and localization of cardiomyopathy locus on chromosome 9qa2.1-b1 using RLGS spot-mapping. Nat Genet (1996) 13:87-90.
Nigro V, Okazaki Y, Belsito A, et al. Identification of the Syrian hamster cardiomyopathy gene. Hum Mol Gen (1997) 6:601-607.
Tagawa H, Koide M, Sato H, Cooper G 4th. Cytoskeletal role in the contractile dysfunction of cardiocytes from hypertrophied and failing right ventricular myocardium. Proc Assoc Am Physicians (1996) 108:218-229.
Kent R. L, Rozich J. D, McCollam P. L, et al. Rapid expression of the Na⁺/Ca²⁺ exchanger in response to cardiac pressure overload. Am J Physiol (1993) 265:H1024-H1029.
Genao A, Seth K, Schmidt U, Carles M, Gwathmey J. K. Dilated cardiomyopathy in turkeys: an animal model for the study of human heart failure. Lab Anim Sci (1996) 46:399-404.
Eschenhagen T, Diederich M, Kluge S. H, et al. Bovine hereditary cardiomyopathy: an animal model of human dilated cardiomyopathy. J Mol Cell Cardiol (1995) 27:357-370.
Rademaker M. T, Charles C. J, Lewis L. K, et al. Beneficial hemodynamic and renal effects of adrenomedullin in an ovine model of heart failure. Circulation (1997) 96:1983-1990.
Rademaker M. T, Charles C. J, Espiner E. A, et al. Natriuretic peptide responses to acute and chronic ventricular pacing in sheep. Am J Physiol (1996) 270:H594-H602.
Aoyagi T, Fujii A. M, Flanagan M. F, et al. Transition from compensated hypertrophy to intrinsic myocardial dysfunction during development of left ventricular pressure-overload hypertrophy in conscious sheep. Systolic dysfunction precedes diastolic dysfunction. Circulation (1993) 88:2415-2425.

Animal models of cardiac hypertrophy

| Species and technique | Selected references |
|---|---|
| Rat | |
| Aortic constriction | Feldman A. M, et al. (1993); |
| | Weinberg E. O, et al. (1994). |
| Pulmonary artery constriction | Julian F. J, et al. (1981). |
| Hypertension | |
| Renal ischemia | Goldblatt H, et al. (1934). |
| DOCA | Besse S, et al. (1994). |
| Dahl salt-sensitive | Dahl L. K, et al. (1962); |
| | Inoko M, et al. (1994). |
| SHR | Okamoto K, et al. (1963); |
| | Bing O. H, et al. (1991). |
| Arteriovenous fistula | Dart C. H Jr., et al. (1969). |
| Hyperthyroidism | Bartosova D, et al. (1969). |
| Hypoxia | Bartosova D, et al. (1969). |
| Catecholamines | Bartosova D, et al. (1969). |
| Exercise | Hickson R. C, et al. (1979); |
| | Rupp H, et al. (1982). |
| Rabbit | |
| Aortic insufficiency/ | Magid N. M, et al. (1994); |
| constriction | Gilson N, et al. (1990); |
| | Ezzaher A, et al. (1991). |
| Pulmonary constriction | Hasenfuss G, et al. (1991). |
| Hyperthyroidism | Hasenfuss G, et al. (1991). |
| Dog | |
| Aortic constriction | Koide M, et al. (1997). |
| Valvular aortic stenosis | Roitstein A, et al. (1995). |
| Tricuspid regurgitation | Dolber P. C, et al. (1994). |
| Pig | |
| Pulmonary artery constriction | Carroll S. M, et al. (1995). |
| Cat | |
| Pulmonary artery constriction | Tagawa H, et al. (1996). |
| Hamster | |
| Genetic | Bajusz E. (1969). |
| Ferret | |
| Pulmonary artery constriction | Do E, et al. (1997); Wang J, et al. (1994). |
| Sheep | |
| Aortic constriction | Charles C. J, et al. (1996). |
| Baboon | |
| Hyperthyroidism | Hoit B. D, et al. (1997). |
| Renal ischemia | Hoit B. D, et al. (1995). |
| Guinea pig | |
| Aortic constriction | Siri F. M, et al. (1989), Siri F. M, et al. |
| | (1991); Kiss E, et al. (1995) , Malhotra A, |
| | et al. (1992), Tweedie D, et al. (1995). |
| Mouse | |
| Renal ischemia | Wiesel P, et al. (1997). |
| Exercise | Kaplan M. L, et al. (1994). |
| Aortic constriction | Dorn G. W 2nd, et al. (1994). |

Feldman A. M, Weinberg E. O, Ray P. E, Lorell B. H. Selective changes in cardiac gene expression during compensated hypertrophy and the transition to cardiac decompensation in rats with chronic aortic banding. Circ Res (1993) 73:184-192.
Weinberg E. O, Schoen F. J, George D, et al. Angiotensin-converting enzyme inhibition prolongs survival and modifies the transition to heart failure in rats with pressure overload hypertrophy due to ascending aortic stenosis. Circulation (1994) 90:1410-1422.
Julian F. J, Morgan D. L, Moss R. L, Gonzalez M, Dwivedi P. Myocyte growth without physiological impairment in gradually induced rat cardiac hypertrophy. Circ Res (1981) 49:1300-1310.
Goldblatt H, Lynch J, Hanzak R. F, Summerville W. W. Studies of experimental hypertension; I. Production of persistent elevation of systolic blood pressure by means of renal ischemia. J Exp Med (1934) 59:347-379.
Besse S, Robert V, Assayag P, Delcayre C, Swynghedauw B. Nonsynchronous changes in myocardial collagen mRNA and protein during aging: effect of DOCA-salt hypertension. Am J Physiol (1994) 267:H2237-2244.
Dahl L. K, Heine M, Tassinari L. Role of genetic factors in susceptibility to experimental hypertension due to chronic excess salt ingestion. Nature (1962) 194:480-482.
Inoko M, Kihara Y, Morii I, Fujiwara H, Sasayama S. Transition from compensatory hypertrophy to dilated, failing left ventricles in Dahl salt-sensitive rats. Am J Physiol (1994) 267:H2471-H2482.
Okamoto K, Aoki K. Development of a strain of spontaneously hypertensive rats. Jpn Circ J (1963) 27:282-293.
Bing O. H, Brooks W. W, Conrad C. H, et al. Intracellular calcium transients in myocardium from spontaneously hypertensive rats during the transition to heart failure. Circ Res (1991) 68:1390-1400.
Dart C. H Jr., Holloszy J. O. Hypertrophied non-failing rat heart; partial biochemical characterization. Circ Res (1969) 25:245-253.
Bartosova D, Chvapil M, Korecky B, et al. The growth of the muscular and collagenous parts of the rat heart in various forms of cardiomegaly. J Physiol (Lond) (1969) 200:285-295.
Hickson R. C, Hammons G. T, Holloszy J. O. Development and regression of exercise-induced cardiac hypertrophy in rats. Am J Physiol (1979) 236:H268-H272.
Rupp H, Jacob R. Response of blood pressure and cardiac myosin polymorphism to swimming training in the spontaneously hypertensive rat. Can J Physiol Pharmacol (1982) 60:1098-1103.
Magid N. M, Opio G, Wallerson D. C, Young M. S, Borer J. S. Heart failure due to chronic experimental aortic regurgitation. Am J Physiol (1994) 267:H556-H562.
Gilson N, el Houda Bouanani N, Corsin A, Crozatier B. Left ventricular function and beta-adrenoceptors in rabbit failing heart. Am J Physiol (1990) 258:H634-H641.
Ezzaher A, Bouanani N. E. H, Su J. B, Hittinger L, Crozatier B. Increased negative inotropic effect of calcium channel blockers in hypertrophied and failing rabbit hearts. J Pharmacol Exp Ther (1991) 257:466-471.
Hasenfuss G, Mulieri L. A, Blanchard E. M, et al. Energetics of isometric force development in control and volume-overload human myocardium. Comparison with animal species. Circ Res (1991) 68:836-846.
Koide M, Nagatsu M, Zile M. R, et al. Premorbid determinants of left ventricular dysfunction in a novel model gradually induced pressure overload in the adult canine. Circulation (1997) 95:1601-1610.
Roitstein A, Chemberg B. V, Kedem J, et al. Reduced effect of phenylephrine on regional myocardial function and O₂ consumption in experimental LVH. Am J Physiol (1995) 268:H1202-H1207.
Dolber P. C, Bauman R. P, Rembert J. C, Greenfield J. C Jr. Regional changes in myocyte structure in model of canine right atrial hypertrophy. Am J Physiol (1994) 267:H1279-H1287.
Carroll S. M, Nimmo L. E, Knoepfler P. S, White F. C, Bloor C. M. Gene expression in a swine model of right ventricular hypertrophy: intercellular adhesion molecule, vascular endothelial growth factor and plasminogen activators are upregulated during pressure overload. J Mol Cell Cardiol (1995) 27:1427-1441.
Tagawa H, Koide M, Sato H, Cooper G 4th. Cytoskeletal role in the contractile dysfunction of cardiocytes from hypertrophied and failing right ventricular myocardium. Proc Assoc Am Physicians (1996) 108:218-229.
Bajusz E. Hereditary cardiomyopathy: a new disease model. Am Heart J (1969) 7:686-696.
Do E, Baudet S, Verdys M, et al. Energy metabolism in normal and hypertrophied right ventricle of the ferret heart. J Mol Cell Cardiol (1997) 29:1903-1913.
Wang J, Flemal K, Qiu Z, et al. Ca²⁺ handling and myofibrillar Ca²⁺ sensitivity in ferret cardiac myocytes with pressure-overload hypertrophy. Am J Physiol (1994) 267:H918-H924.
Charles C. J, Kaaja R. J, Espiner E. A, et al. Natriuretic peptides in sheep with pressure overload left ventricular hypertrophy. Clin Exp Hypertens (1996) 18:1051-1071.
Hoit B. D, Pawloski-Dahm C. M, Shao Y, Gabel M, Walsh R. A. The effects of a thyroid hormone analog on left ventricular performance and contractile and calcium cycling proteins in the baboon. Proc Assoc Am Physicians (1997) 109:136-145.
Hoit B. D, Shao Y, Gabel M, Walsh R. A. Disparate effects of early pressure overload hypertrophy on velocity-dependent and force-dependent indices of ventricular performance in the conscious baboon. Circulation (1995) 91:1213-1220.
Siri F. M, Nordin C, Factor S. M, Sonnenblick E, Aronson R. Compensatory hypertrophy and failure in gradual pressure-overloaded guinea pig heart. Am J Physiol (1989) 257:H1016-H1024.
Siri F. M, Krueger J, Nordin C, Ming Z, Aronson R. S. Depressed intracellular calcium transients and contraction in myocytes from hypertrophied and failing guinea pig hearts. Am J Phys (1991) 261:H514-H530.
Kiss E, Ball N. A, Kranias E. G, Walsh R. A. Differential changes in cardiac phospholamban and sarcoplasmic reticulum Ca²⁺-ATPase protein levels. Effects on Ca²⁺ transport and mechanics in compensated pressure-overload hypertrophy and congestive heart failure. Circ Res (1995) 77:759-764.
Malhotra A, Siri F. M, Aronson R. Cardiac contractile proteins in hypertrophied and failing guinea pig heart. Cardiovasc Res (1992) 26:153-161.
Tweedie D, Henderson C. G, Kane K. A. Assessment of subrenal banding of the abdominal aorta as a method of inducing cardiac hypertrophy in the guinea pig. Cardioscience (1995) 6:115-119.
Wiesel P, Mazzolai L, Nussberger J, Pedrazzini T. Hypertension. (1997) 29:1025-1030.
Kaplan M. L, Cheslow Y, Vikstrom K, et al. Cardiac adaptations to chronic exercise in mice. Am J Physiol (1994) 267:H1167-H1173.
Dorn G. W 2nd, Robbins J, Ball N, Walsh R. A. Myosin heavy chain regulation and myocyte contractile depression after LV hypertrophy in aortic-banded mice. Am J Physiol (1994) 267:H400-H405.

Transgenic models of heart failure and hypertrophy

| Intervention | Phenotype | Reference |
|---|---|---|
| Gene overexpression | | |
| C-myc | Myocardial hyperplasia | Jackson T, et al. (1990) |
| Epstein-Barr virus | Dilated cardiomyopathy | Huen D. S, et al. (1993). |
| nuclear antigen | | |
| Polyomavirus large | Cardiomyopathy | Chalifour L. E, et al. |
| T-antigen | | (1990). |
| Calmodulin | Myocardial hypertrophy | Gruver C. L, et al. (1993). |
| | and hyperplasia | |
| Myogenic factor 5 | Cardiomyopathy and | Edwards J. G, et al. (1996). |
| | Failure | |
| Gₛ α | Cardiomyopathy and | Iwase M, et al. (1997). |
| | Failure | |
| α₁-Adrenergic | Myocardial hypertrophy | Milano C. A, et al. (1994). |
| receptor | | |
| p21-ras | Myocardial hypertrophy; | Hunter J. J, et al. (1995). |
| | myofibrillar disarray | |
| Interleukin β and | Hypertrophy | Hirota H, et al. (1995). |
| interleukin β | | |
| receptor | | |
| Nerve growth | Cardiomyopathy | Hassankhani A, et al. |
| factor | | (1995). |
| Insulin-like | Cardiomyopathy; | Reiss K, et al. (1995). |
| growth factor 1 | Hyperplasia | |
| β-adrenergic | Reduced contractility | Rockman H. A, et al. |
| receptor Kinase | | (1995) |
| G protein coupled | Reduced contractility | Bertin B, et al. (1993). |
| receptor kinase | | |
| TGR (m Ren 2)27 | Hypertrophy in rats | Langheinrich M, et al. |
| | | (1996). |
| Gene mutation | | |
| α-cardiac myosin | Hypertrophic | Geisterfer-Lowrance |
| heavy | | A. A. T, et al. (1996). |
| Chain | Cardiomyopathy | |
| Lack of β-myosin | Hypertrophic | Welikson R. E, et al. (1997). |
| light | | |
| chain binding | Cardiomyopathy | |
| domain | | |
| Knockout of gene | | |
| Muscle LIM | Dilated cardiomyopathy | Arber S, et al. (1997). |
| protein | and failure | |
| Adenine nucleotide | Hypertrophy | Graham B. H, et al. (1997). |
| Translocator | | |
| Transforming | Myocarditis and failure | Shull M. M, et al. (1992). |
| growth factor β | | |
| Interferon | Myocarditis and failure | Aitken K, et al. (1994). |
| regulatory | | |
| factor 1 | | |

Jackson T, Allard M. F, Sreenan C. M, et al. The c-myc proto-oncogene regulates cardiac development in transgenic mice. Mol Cell Biol (1990) 10:3709-3716.
Huen D. S, Fox A, Kumar P, Searle P. F. Dilated heart failure in transgenic mice expression the Epstein-Barr virus nuclear antigen-leader protein. J Gen Virol (1993) 74:1381-1391.
Chalifour L. E, Gomes M. L, Wang N. S, Mes Masson A. M. Polyomavirus large T-antigen expression in heart of transgenic mice causes cardiomyopathy. Oncogene (1990) 5:1719-1726.
Gruver C. L, DeMayo F, Goldstein M. A, Means A. R. Targeted developmental overexpression of calmodulin induces proliferative and hypertrophic growth of cardiomyocytes in trangenic mice. Endocrinology (1993) 133:376-388.
Edwards J. G, Lyons G. E, Micales B. K, Malhotra A, Factor S, Leinwand L. A. Cardiomyopathy in trangenic myf5 mice. Circ Res (1996) 78:379-387.
Iwase M, Uechi M, Vatner D. E, et al. Cardiomyopathy induced by cardiac Gs alpha overexpression. Am J Phys (1997) 272:H585-H589.
Milano C. A, Dolber P. C, Rockman H. A, et al. Myocardial expression of a constitutively active α1b-adrenergic receptor in trangenic mice induces cardiac hypertrophy. Proc Natl Acad Sci USA (1994) 91:10109-10113.
Hunter J. J, Tanaka N, Rockman H. A, Ross J, Chien K. R. Ventricular expression of a MLC-2v-ras fusion gene induces cardiac hypertrophy and selective diastolic dysfunction in transgenic mice. J Biol Chem (1995) 270:23173-23178.
Hirota H, Yoshida K, Kishimoto T, Taga T. Continuous activation of gp130, a signal-retransducing receptor component for interleukin 6-related cytokines, cause myocardial hypertrophy in mice. Proc Natl Acad Sci USA (1995) 92:4862-4866.
Hassankhani A, Steinhelper M. E, Soonpaa M. H, et al. Overexpression of NGF within the heart of transgenic mice causes hyperinnervation, cardiac enlargement, and hyperplasia of ectopic cells. Dev Biol (1995) 169:309-321.
Reiss K, Cheng W, Ferber A, et al. Overexpression of IGF-1 in the heart is coupled with myocyte proliferation in transgenic mice. Circulation (1995) 92(Suppl I):370.
Rockman H. A, Hamilton R, Rahman N. U, et al. Dampened cardiac function in vivo in transgenic mice overexpression GRK5, a G protein-coupled receptor kinase. Circulation (1995) 92(Suppl I):240.
Bertin B, Mansier P, Makeh I, et al. Specific atrial over-expression of G protein coupled human β₁-adrenoceptors in transgenic mice. Cardiovasc Res (1993) 27:1606-1612.
Langheinrich M, Lee M. A, Bohm M, et al. The hypertensive Ren-2 transgenic rat TGR (mREN2)27 in hypertension research. Characteristics and functional aspects. Am J Hypertens (1996) 9:506-512.
Geisterfer-Lowrance A. A. T, Christe M, Conner D. A, et al. A mouse model of familial hypertrophic cardiomyopathy. Science (1996) 272:731-734.
Welikson R. E, Vikstrom K. L, Factor S. M, Weinberger H. D, Leinwand L. A. Heavy chains lacking the light chain binding domain cause genetically dominant cardiomyopathy in mice. Circulation (1997) 96(Suppl I):571.
Arber S, Hunter J. J, Ross J Jr., et al. MLP-deficient mice exhibit a disruption of cardiac cytoarchitectural organization, dilated cardiomyopathy, and heart failure. Cell (1997) 88:393-403.
Graham B. H, Waymire K. G, Cottrell B, et al. A mouse model for mitochondrial myopathy and cardiomyopathy resulting from a deficiency in the heart/muscle isoform of the adenine nucleotide translocator. Nat Genet (1997) 16:226-234.
Shull M. M, Ormsby I, Kier A. B, et al. Targeted disruption of the mouse tranforming growth factor-β1 gene results in multifocal inflammatory disease. Nature (1992) 359:693-699.
Aitken K, Penninger J, Mak T, et al. Increased susceptibility to coxsackie viral myocarditis in IRF-1 transgenic knockout mice. Circulation (1994) 90(Suppl I):139.

### B. NABTs for the Treatment of Vascular Disorders

Atherosclerosis is a condition in which vascular smooth muscle cells are pathologically reprogrammed. Fatty material collects in the walls of arteries and there is typically chronic inflammation. This leads to a situation where the vascular wall thickens, hardens, forms plaques, which may eventually block the arteries or promote the blockage of arteries by promoting clotting. The latter becomes much more prevalent when there is a plaque rupture.

If the coronary arteries become narrow due to the effects of the plaque formation, blood flow to the heart can slow down or stop, causing chest pain (stable angina), shortness of breath, heart attack, and other symptoms. Pieces of plaque can break apart and move through the bloodstream. This is a common cause of heart attack and stroke. If the clot moves into the heart, lungs, or brain, it can cause a stroke, heart attack, or pulmonary embolism.

Risk factors for atherosclerosis include: diabetes, high blood pressure, high cholesterol, high-fat diet, obesity, personal or family history of heart disease and smoking. The following conditions have also been linked to atherosclerosis: cerebrovascular disease, kidney disease involving dialysis and peripheral vascular disease. Down modulation of a variety of genes can have a beneficial therapeutic effect for the treatment of artherosclerosis and associated pathologies. These are listed in Table 11 and include, without limitation, androgen receptor, c-myb, DB-1, DP-1, E2F-1, ERG-1, FLT-4, ICH-1L, ISGF3, NF-IL6, OCT-1, p53, Sp-1, PDEGF, and PDGFR. WO/2007/030556 provides an animal model for assessing the effects of modified NABTs directed to the aforementioned targets on the formation of atherosclerotic lesions. NABTs targeting the genes listed above will be prepared with modified backbones, as described elsewhere.

Atherosclerotic plaque rupture is the main cause of coronary thrombosis and myocardial infarcts. Rekhter et al. have developed a rabbit model in which an atherosclerotic plaque can be ruptured at will after an inflatable balloon becomes embedded into the plaque. Furthermore, the pressure needed to inflate the plaque-covered balloon may be an index of overall plaque mechanical strength (Circulation Research. 1998; 83:705-713). The thoracic aorta of hypercholesterolemic rabbits underwent mechanical removal of endothelial cells, and then a specially designed balloon catheter was introduced into the lumen of the thoracic aorta. As early as 1 month after catheter placement, atherosclerotic plaque formed around the indwelling balloon. The plaques were reminiscent of human atherosclerotic lesions, in terms of cellular composition, patterns of lipid accumulation, and growth characteristics. Intraplaque balloons were inflated both ex vivo and in vivo, leading to plaque fissuring. The ex vivo strategy is designed to measure the mechanical strength of the surrounding plaque, while the in vivo scenario permits an analysis of the plaque rupture consequences, eg, thrombosis. This model can be used to advantage for assessing local delivery of the NABTs described herein into the plaque in order to assess the effects of the same on plaque instability.

### Example 2

### Brain Cell Directed NABTs and Methods of Use Thereof for the Treatment of Alzheimer's Disease and Other Neurological Disorders

### A. Alzheimer's Disease

NABTs directed to particular targets in neurological cells have efficacy for the treatment of Alzheimer's Disease and other neurological disorders. Suitable targets for treatment of Alzheimer's Disease include without limitation, apolipoprotein epsilon 4, β amyloid precursor protein, CDK-2, Cox-2, CREB, CREBP, Cyclin B, ICH-1L (also known as caspase 2L), PKC genes, PDGFR, SGP2, SRF, and TRPM-2

The amyloid hypothesis postulates that Alzheimer's Disease is caused by aberrant production or clearance of the amyloid β (Aβ) peptide from the brains of affected individuals. Aβ is toxic to neurons and forms plaques in the brains of Alzheimer's Disease patients. These plaques constitute one of the hallmark pathologies of the disease. Aβ is produced by the consecutive proteolytic cleavage of the Amyloid Precursor Protein (APP) by β-secretase (BACE) and γ-secretase proteases. APP is also cleaved by α-secretase but this process generates non-amyloidogenic products. Cleavage by γ-secretase generates Aβ peptides of variable lengths. The 42 amino acid form of Aβ (Aβ1-42) is known to be the most toxic.

The NABTs of the invention can be incubated with a neuronal cell line, e.g., ELLIN a human neuroblastoma cell line which produces detectable levels of Aβ. The effect of the NABT on Aβ production can be readily determined using conventional biochemical methods. This cell line is suitable for characterizing the NABTs of the invention which modulate endogenous AP production. The cells are deposited at the ECACC under depositor reference ELLIN as cell line BE(2)-C. BE(2)-C (ECACC #95011817) is a clonal sub-line of SK-N-BE(2) (ECCAC #95011815) which was isolated from bone marrow of an individual with disseminated neuroblastoma in 1972. They are reported to be multipotential with regard to neuronal enzyme expression and display a high capacity to convert tyrosine to dopamine. The cells show a small, refractile morphology with short, neurite-like cell processes and tend to grow in aggregates. See WO/2008/084254 entitled “Cell line for Alzheimers's disease therapy screening” which is incorporated herein by reference.

Also suitable for screening are clonal cell lines derived by fusion of dorsal root ganglia neurons with neuroblastoma cells as described in Platika et al., PNAS (1985) 82:3499-3503. These cells have been immortalized and retain their neuronal phenotype and thus also have utility for screening the nucleic acid based therapeutics of the invention for their ability to modulate neuronal structure and function.

The table below provides art recognized rodent models for optimizing modifications of the NABTs described herein for the treatment and/or prevention of Alzheimer's Disease. Methods for assessing: 1) the formation of abnormal plaques in the brain; 2) neuronal loss, and 3) the development of diminished cognitive function and memory loss are readily assessed in animal models described in the cited references.

As set forth in Spires et al. (2005) NeuroRx 2: 423-437), Games and colleagues (Nature 373: 523-527, 1995) reported a convincing mouse model of AD, the PDAPP mouse, in 1995. PDAPP mice overexpress human APP cDNA with portions of APP introns 6-8 and with valine at residue 717 substituted by phenalalanine—one of the FAD-associated mutations—under the control of a platelet-derived growth factor β (PDGFβ) promoter. These mice, unlike the earlier APP models controlled by an NSE promoter, express very high levels of APP protein (˜10-fold higher than endogenous APP), and they develop more Alzheimer-like neuropathology, including extracellular diffuse and neuritic plaques, dystrophic neurites, gliosis, and loss of synapse density. Notably, plaque formation in these mice proceeds from the hippocampus (at 6-8 months) to cortical and limbic areas (8 months) in a progressive manner showing regional specificity like that seen in AD pathology. Furthermore, amyloid burden and memory impairment assessed using a modified Morris water maze task increase with aging. The amyloid pathology in PDAPP mice is strikingly similar to that observed in AD. Ultrastructural comparisons reveal similar amyloid fibril size, similar plaque-associated dystrophic neurites containing synaptic components and neurofibrils, association of microglia with plaques, and phosphorylation of neurofilaments and tau protein in neurites in aged mice (18 months). However, these neurodegenerative alterations are not accompanied by paired-helical filament formation, and stereological analysis by Irizarry et al. revealed no global neuronal loss in the entorhinal cortex, CA1, or cingulate cortex through 18 months of age. Loss of neurons in the immediate vicinity of dense-cored plaques, however, was observed mimicking observations in human AD.

In 1996, Hsiao et al. published another APP overexpressing mouse model of AD, the Tg2576 line (Science 274: 99-102, 1996). These mice are transgenic for human APP cDNA with the double Swedish mutation (K670N and M671 L) under the control of the hamster prion protein promoter (PrP). Heterozygous Tg2576 mice produce APP at 5.5-fold over endogenous levels and develop diffuse and neuritic plaques in the hippocampus, cortex, subiculum, and cerebellum at around 9-11 months of age similar to those seen in AD and PDAPP mice. In spontaneous alternation and water maze tasks, Tg2576 mice show subtle age-related memory deficits starting at around 8 months of age. They also have an age-dependent electrophysiological phenotype at older ages characterized by impaired induction of LTP in the hippocampus in vitro and in vivo. In cortex, synaptic integration is also disrupted in vivo. These functional disruptions may underlie some of the observed memory deficits. Plaques in Tg2576 mice are associated with dystrophic neurites and gliosis, but without global loss of synapses or neurons in CA1.

Lanz et al. reported that dendritic spine density decreases in CA1 of both PDAPP and Tg2567 mice before plaque deposition, demonstrating that these models both emulate some of the disrupted synaptic circuitry seen in AD (Neurobiol Dis 13: 246-253, 2003). APP23 mice, developed at Novartis, overexpress human APP cDNA with the Swedish mutation under control of the murine Thy1.2 promoter. These mice develop both amyloid plaques and cerebral amyloid angiopathy starting at around 6 months of age. Similarly to the previously described models, APP23 mice develop memory deficits as assessed by behavioral tests. Unlike the PDAPP and Tg2576 lines, neuron loss of 14% was reported in the CA1 of the APP23 mice, although no loss was detected in the cortex.

Another APP overexpressing mouse line with the Swedish mutation, developed by Borchelt et al. does not develop plaques until 18 months (line APP Swe C3-3) (Neuron 19: 939-945, 1997). The transgene is driven by a different promoter (mouse prion promoter) and is on a different background strain (C57BL/6-C3H) from the Tg2576 and APP23 models mentioned above that have earlier onset of amyloid deposition. Expression of both the Swedish mutation and the V717F mutation driven by the Syrian hamster prion promoter (TgCRND8 mouse model) causes early deposition of amyloid in plaques and premature death dependent on background strain, indicating the importance of genetic background on the effects of APP overexpression. TgCRND8 mice also perform poorly in the water maze indicating memory deficits.

Several different animal models for assessing modifications to the NABTs described herein are provided in the table below.

| | | | Neuro- | | | | | | | |
|---|---|---|---|---|---|---|---|---|---|---|
| | Gene(s) | | pathology | P- | | Cell | Memory | Age of Onset | | |
| Name | Overexpressed | Promoter | Plaques | tau | NFT | Loss | Deficits | (of Pathology) | | Ref. |
| PDAPP mice | APP minigene, V717F | PDGFβ | Yes | Yes | No | No | Yes | 6-8 | months | Games D, et al. (1995); |
| | mutation | | | | | | | | | Masliah E, et al.(1996); |
| | | | | | | | | | | Irizarry M C, (1997); |
| | | | | | | | | | | Chen G, et al. (2000). |
| Tg2576 mice | APP Swe cDNA (695) | Hamster PrP | Yes | Yes | No | No | Yes | 9-11 | Months | Hsiao K, et al (1996); |
| | | | | | | | | | | Irizarry M C, et al. |
| | | | | | | | | | | (1997); Lanz T A, et al. |
| | | | | | | | | | | (2003). |
| APP23 mice | APP Swe cDNA (751) | Murine Thy1 | Yes | Yes | No | Yes | Yes | 6 | Months | Sturchler-Pierrat C, et |
| | | | | | | (CA1) | | | | al. (1997); Calhoun M E, |
| | | | | | | | | | | et al. (1998). |
| TgCRND8 mice | APP cDNA Swe and | Syrian hamster | Yes | Nr | No | nr | Yes | 3 | Months | Dudal S, et al. (2004); |
| | V717F mutations | PrP | | | | | | | | Chishti M A, et al. |
| | | | | | | | | | | (2001). |
| APPSwe | APP cDNA (695) Swe | Murine PrP | Yes | Nr | Nr | nr | nr | 18 | Months | Borchelt D R, et al. |
| TgC3-3 mice | | | | | | | | | | (1997); Borchelt D R, et |
| | | | | | | | | | | al. (1996). |
| PSAPP mice | Tg2576 and PSI M146L | Hamster PrP, | Yes | Yes | Nr | Minor | Yes | 6 | Months | Holcomb L, et al. |
| | | PDGFβ | | | | | | | | (1998); Holcomb L A, et |
| | | | | | | | | | | al. (1999). |
| Tg478/1116/ | APP Swe, APP Swe and | Rat synapsin 1, | Yes | Nr | Nr | nr | nr | 9 | Months | Flood D G, et al. (2003). |
| 11587 rat | V717F, PS1, M146V | PDGFβ, Rat | | | | | | | | |
| | | synapsin I | | | | | | | | |
| ALZ7 mice | 4R tau | Human Thy1 | No | Yes | No | No | nr | — | | Gotz J, et al. (1995). |
| ALZ17 mice | 4R tau | Murine Thy1 | No | Yes | No | No | nr | — | | Probst A, et al. (2000). |
| 7TauTg mice | 3R tau | Murine PrP | No | Yes | Yes | nr | nr | 18-20 | Months | Ishihara T, et al. (2001). |
| JNPL3 mice | 4R tau P301L | Murine PrP | No | Yes | Yes | Yes | Yes | 5 | Months | Lewis J, et al. (2000); |
| | | | | | | | | | | Arendash G W, et al. |
| | | | | | | | | | | (2004). |
| pR5 mice | 4R tau P301L | Murine Thy1 | No | Yes | Yes | Yes | nr | 8 | Months | Gotz J, et al. (2001). |
| TAPP mice | Tg2576x JNPL3 | Hamster PrP, | Yes | Yes | Yes | nr | nr | 6 | Months | Lewis J, et al. (2001). |
| | | Murine PrP | | | | | | | | |
| 3xTg-AD | APP (Swe), PS1 | Murine Thy1 | Yes | Yes | Yes | nr | nr | 3 | Months | Oddo S, et al. (2003); |

| | | |
|---|---|---|
| (M146V), tau (P301L) | (PS1 knockin) | Oddo S, et al. (2003). |

nr = not reported;
Swe = Swedish mutation;
P-tau = 32 phosphorylated tau immunoreactivity.
NeuroRx. 2005

Games D, Adams D, Alessandrini R, Barbour R, Berthelette P, Blackwell C, et al. Alzheimer-type neuropathology in transgenic mice overexpressing V717F β-amyloid precursor protein. Nature 373: 523-527, 1995
Masliah E, Sisk A, Mallory M, Mucke L, Schenk D, Games D. Comparison of neurodegenerative pathology in transgenic mice overexpressing V717F β-amyloid precursor protein and Alzheimer's disease. J Neurosci 16: 5795-5811, 1996.
Irizarry M C, Soriano F, McNamara M, Page K J, Schenk D, Games D, et al. Aβ deposition is associated with neuropil changes, but not with overt neuronal loss in the human amyloid precursor protein V717F (PDAPP) transgenic mouse. J Neurosci 17: 7053-7059, 1997.
Chen G, Chen K S, Knox J, Inglis J, Bernard A, Martin S J, et al. A learning deficit related to age and β-amyloid plaques in a mouse model of Alzheimer's disease. Nature 408: 975-979, 2000.
Hsiao K, Chapman P, Nilsen S, Eckman C, Harigaya Y, Younkin S, et al. Correlative memory deficits, Aβ elevation, and amyloid plaques in transgenic mice. Science 274: 99-102, 1996.
Irizarry M C, McNamara M, Fedorchak K, Hsiao K, Hyman B T. APPSw transgenic mice develop age-related A β deposits and neuropil abnormalities, but no neuronal loss in CA1. J Neuropathol Exp Neurol 56: 965-973, 1997.
Lanz T A, Carter D B, Merchant K M. Dendritic spine loss in the hippocampus of young PDAPP and Tg2576 mice and its prevention by the ApoE2 genotype. Neurobiol Dis 13: 246-253, 2003.
Sturchler-Pierrat C, Abramowski D, Duke M, Wiederhold K H, Mistl C, Rothacher S, et al. Two amyloid precursor protein transgenic mouse models with Alzheimer disease-like pathology. Proc Natl Acad Sci USA 94: 13287-13292, 1997.
Calhoun M E, Wiederhold K H, Abramowski D, Phinney A L, Probst A, Sturchler-Pierrat C, et al. Neuron loss in APP transgenic mice. Nature 395: 755-756, 1998.
Borchelt D R, Ratovitski T, van Lare J, Lee M K, Gonzales V, Jenkins N A, et al. Accelerated amyloid deposition in the brains of transgenic mice coexpressing mutant presenilin 1 and amyloid precursor proteins. Neuron 19: 939-945, 1997.
Borchelt D R, Thinakaran G, Eckman C B, Lee M K, Davenport F, Ratovitsky T, et al. Familial Alzheimer's disease-linked presenilin 1 variants elevate Aβ1-42/1-40 ratio in vitro and in vivo. Neuron 17: 1005-1013, 1996.
Dudal S, Krzywkowski P, Paquette J, Morissette C, Lacombe D, Tremblay P, et al. Inflammation occurs early during the Aβ deposition process in TgCRND8 mice. Neurobiol Aging 25: 861-871, 2004.
Chishti M A, Yang D S, Janus C, Phinney A L, Horne P, Pearson J, et al. Early-onset amyloid deposition and cognitive deficits in transgenic mice expressing a double mutant form of amyloid precursor protein 695. J Biol Chem 276: 21562-21570, 2001.
Holcomb L, Gordon M N, McGowan E, Yu X, Benkovic S, Jantzen P, et al. Accelerated Alzheimer-type phenotype in transgenic mice carrying both mutant amyloid precursor protein and presenilin 1 transgenes. Nat Med 4: 97-100, 1998.
Holcomb L A, Gordon M N, Jantzen P, Hsiao K, Duff K, Morgan D. Behavioral changes in transgenic mice expressing both amyloid precursor protein and presenilin-1 mutations: lack of association with amyloid deposits. Behav Genet. 29: 177-185, 1999.
Flood D G, Howland D S, Lin Y-G, Ciallella J R, Trusko S P, Scott R W, Savage M S. Aβ deposition in a transgenic rat model of Alzheimer's disease. Poster 842.22 presented at Society for Neuroscience meeting, New Orleans, La., 2003.
Gotz J, Probst A, Spillantini M G, Schafer T, Jakes R, Burki K, et al. Somatodendritic localization and hyperphosphorylation of tau protein in transgenic mice expressing the longest human brain tau isoform. EMBO J 14: 1304-1313, 1995.
Probst A, Gotz J, Wiederhold K H, Tolnay M, Mistl C, Jaton A L, et al. Axonopathy and amyotrophy in mice transgenic for human four-repeat tau protein. Acta Neuropathol (Berl) 99: 469-481, 2000.
Ishihara T, Zhang B, Higuchi M, Yoshiyama Y, Trojanowski J Q, Lee V M. Age-dependent induction of congophilic neurofibrillary tau inclusions in tau transgenic mice. Am J Pathol 158: 555-562, 2001.
Lewis J, McGowan E, Rockwood J, Melrose H, Nacharaju P, Van Slegtenhorst M, et al. Neurofibrillary tangles, amyotrophy and progressive motor disturbance in mice expressing mutant (P301L) tau protein. Nat Genet. 25: 402-405, 2000.
Arendash G W, Lewis J, Leighty R E, McGowan E, Cracchiolo J R, Hutton M, et al. Multi-metric behavioral comparison of APPsw and P301L models for Alzheimer's disease: linkage of poorer cognitive performance to tau pathology in forebrain. Brain Res 1012: 29-41, 2004.
Gotz J, Chen F, Barmettler R, Nitsch R M. Tau filament formation in transgenic mice expressing P301L tau. J Biol Chem 276: 529-534, 2001.
Lewis J, Dickson D W, Lin W L, Chisholm L, Corral A, Jones G, et al. Enhanced neurofibrillary degeneration in transgenic mice expressing mutant tau and APP. Science 293: 1487-1491, 2001.
Oddo S, Caccamo A, Shepherd J D, Murphy M P, Golde T E, Kayed R, et al. Triple-transgenic model of Alzheimer's disease with plaques and tangles: intracellular AP and synaptic dysfunction. Neuron 39: 409-421, 2003
Oddo S, Caccamo A, Kitazawa M, Tseng B P, LaFerla F M. Amyloid deposition precedes tangle formation in a triple transgenic model of Alzheimer's disease. Neurobiol Aging 24: 1063-1070, 2003.

### B. Multiple Sclerosis

Multiple sclerosis (abbreviated MS, also known as disseminated sclerosis or encephalomyelitis disseminata) is an autoimmune condition characterized by demyelination. Disease onset usually occurs in young adults, and it is more common in females. It has a prevalence that ranges between 2 and 150 per 100,000. MS was first described in 1868 by Jean-Martin Charcot.

MS affects the ability of nerve cells in the brain and spinal cord to communicate with each other. Nerve cells communicate by sending electrical signals called action potentials down long fibers called axons, which are wrapped in an insulating substance called myelin. When myelin is lost, the axons can no longer effectively conduct signals. The name multiple sclerosis refers to scars (scleroses—better known as plaques or lesions) in the white matter of the brain and spinal cord, which is mainly composed of myelin. Although much is known about the mechanisms involved in the disease process, the cause remains unknown. Theories include genetics or infections. Different environmental risk factors have also been found.

Almost any neurological symptom can appear with the disease which often progresses to physical and cognitive disability. MS takes several forms, with new symptoms occurring either in discrete attacks (relapsing forms) or slowly accumulating over time (progressive forms). Between attacks, symptoms may go away completely, but permanent neurological problems often occur, especially as the disease advances.

There is no known cure for MS. Existing treatments attempt to return function after an attack, prevent new attacks, and prevent disability. MS medications can have adverse effects or be poorly tolerated, and many patients pursue alternative treatments, despite the lack of supporting scientific study. The prognosis is difficult to predict; it depends on the subtype of the disease, the individual patient's disease characteristics, the initial symptoms and the degree of disability the person experiences as time advances. Life expectancy of patients is nearly the same as that of the unaffected population, nonetheless, improved therapeutic agents for the treatment of multiple sclerosis are urgently needed. Several of the NABTs of the invention target molecules which are causally implicated in MS. These include, without limitation, COX-2, p53, TNF-α, and TNF-β. Accordingly, administration of NABTs targeting such molecules should exhibit beneficial therapeutic effects to patients in need of such treatment. In a preferred embodiment, NABTs which inhibit p53 expression can be delivered nasally to reduce the pathological symptoms associated with MS.

U.S. Pat. No. 7,423,194 provides an animal model and cells suitable for assessing the effect of modified NABTs described herein on demyelination.

Different models of experimental autoimmune encephalomyelitis (EAE) have also been successfully applied to investigate aspects of the autoimmune pathogenesis of multiple sclerosis. See Wekerle et al. Annals of Neurology (2004) 36: (S1), S47-S53). Studies using myelin-specific T-cell lines that transfer EAE to naive recipient animals established that only activated lymphocytes are able to cross the endothelial blood-brain barrier and cause autoimmune disease within the local parenchyma. All encephalitogenic T cells are CD4⁺ Th1-type lymphocytes that recognize autoantigenic peptides in the context of MHC class II molecules. In the case of myelin basic protein (MBP) specific EAE in the Lewis rat, the T-cell response is directed against one strongly dominant peptide epitope. The encephalitogenic T cells preferentially use one particular set of T-cell receptor genes. Although MBP is a strong encephalitogen in many species, a number of other brain proteins are now known to induce EAE. These include mainly myelin components (PLP, MAG, and MOG), but also, the astroglial S-100β protein. Encephalitogenic T cells produce only inflammatory changes in the central nervous system, without extensive primary demyelination. Destruction of myelin and oligodendrocytes in these models requires additional effector mechanisms such as auto-antibodies binding to myelin surface antigens such as the myelin-oligodendrocyte glycoprotein. This animal model may also be used to advantage to assess the effects of the NABTs described above on demyelination processes.

### C. Parkinson's Disease

Parkinson's disease is a chronic, progressive neurodegenerative movement disorder. Tremors, rigidity, slow movement (bradykinesia), poor balance, and difficulty walking (called parkinsonian gait) are characteristic primary symptoms of Parkinson's disease. Parkinson's disease afflicts 1 to 1½ million people in the United States. The disorder occurs in all races but is somewhat more prevalent among Caucasians. Men are affected slightly more often than women. Symptoms of Parkinson's disease may appear at any age, but the average age of onset is 60. It is rare in people younger than 30 and risk increases with age. It is estimated that 5% to 10% of patients experience symptoms before the age of 40. Parkinson's disease is common in the elderly and one in 20 people over the age of 80 has the condition.

Parkinson's results from the degeneration a number of nuclei in the dopamine-producing nerve cells in the brainstem. Most attention has been given to the substantia nigra and the locus coeruleus. Dopamine is a neurotransmitter that stimulates motor neurons, those nerve cells that control the muscles. When dopamine production is depleted, the motor system nerves are unable to control movement and coordination. Parkinson's Disease (PD) patients have lost 80% or more of their dopamine-producing cells by the time symptoms appear.

Clearly, there is an urgent need for new and improved therapeutic agents for the treatment of Parkinson's disease. Such a need is met by the NABTs specific for several gene targets relevant for the treatment of Parkinson's Disease described herein. These include, without limitation, COX-2, FAS/APO-1, p53, and PKC gamma.

Teismann et al. have shown that COX-2 for example, the rate-limiting enzyme in prostaglandin E₂ synthesis, is up-regulated in brain dopaminergic neurons of both PD and MPTP mice (PNAS (2003) 100:5473-5478. COX-2 induction occurs through a JNK/c-Jun-dependent mechanism after MPTP administration. Targeting COX-2 does not protect against MPTP-induced dopaminergic neurodegeneration by mitigating inflammation. Evidence is provided showing COX-2 inhibition prevents the formation of the oxidant species dopamine-quinone, which has been implicated in the pathogenesis of PD. This study supports a critical role for COX-2 in both the pathogenesis and selectivity of the PD neurodegenerative process. There are safety concerns connected to the use of certain currently available COX-2 inhibitors. NABTs directed to COX-2 should have efficacy for the treatment of this disorder. NABTs modified to include a carrier which improves their capacity to penetrate the blood brain barrier as described herein can be useful therapeutics for the treatment of PD. Such NABTs can be further characterized in any of the current models for PD (e.g. the reserpine model, neuroleptic-induced catalepsy, tremor models, experimentally-induced degeneration of nigro-striatal dopaminergic neurons with 6-OHDA, methamphetamine, MPTP, MPP⁺, tetrahydroisoquinolines, β-carbolines, and iron) as described by Gerlach et al. J. of Neural Transmission 103:987:1041.

Programmed cell death plays an important role in the neuronal degeneration after cerebral ischemia, but the underlying mechanisms are not fully understood. Martin-Villalba et al. examined, in vivo and in vitro, whether ischemia-induced neuronal death involves death-inducing ligand/receptor systems such as CD95 (Fas-L/APO-1L) and tumor necrosis factor-related apoptosis-inducing ligand (TRAIL). After reversible middle cerebral artery occlusion in adult rats, both CD95 ligand and TRAIL were expressed in the apoptotic areas of the postischemic brain. Further recombinant CD95 ligand and TRAIL proteins induced apoptosis in primary neurons and neuron-like cells in vitro. The immunosuppressant FK506, which protects cells against ischemic neurodegeneration, prevented post-ischemic expression of these death-inducing ligands both in vivo and in vitro. FK506 also abolished phosphorylation, but not expression, of the c-Jun transcription factor involved in the transcriptional control of CD95 ligand. Most importantly, in 1pr mice expressing dysfunctional CD95, reversible middle cerebral artery occlusion resulted in infarct volumes significantly smaller than those found in wild-type animals. These results suggest an involvement of CD95 ligand and TRAIL in the pathophysiology of postischemic neurodegeneration and offer alternative strategies for the treatment of cardiovascular brain disease. See Martin Villaba et al. (1999) J. of Neuroscience 19:3809-3817.

Thus, NABTs which selectively down modulate FAS/APO-1 provided herein should have efficacy for the treatment of disorders associated with aberrant neuronal cell apoptosis, such as Parkinson's Disease, Alzheimer's Disease, Huntingon's disease etc. Such NABTs can be assessed in the various cell line and animal models described in the present example.

p53, Bax and Bcl-X_{L} proteins have been implicated in apoptotic neuronal cell death. Blum et al. investigated whether those proteins are involved in 6-OHDA-induced PC12 cell death. After a 24-h exposure to the neurotoxin (100 μM), morphological evidence for apoptosis was observed in PC12 cells. Up-regulation of p53 and Bax proteins was demonstrated 4 and 6 h, respectively, after 6-OHDA treatment; in contrast, no change in Bcl-X_{L}, levels was found. These findings suggest that p53 provides a relevant marker of neuronal apoptosis as previously described in kainic acid- or ischemia-induced neuronal cell death and may participate to neuronal degeneration in Parkinson's Disease. Brain Research (1997) 751:139-142. This model system is also useful for assessing the efficacy of the p53 directed NABTs and modifications thereto as described above for the treatment of Huntington's disease.

### Example 3

### Anti-Cancer NABTs and Methods of Use Thereof for the Treatment of Neoplastic and Hyper-Proliferative Diseases

### A. Anti-Cancer NABTs and Methods of Use Thereof.

Cellular transformation during the development of cancer involves multiple alterations in the normal pattern of cell growth regulation and dysregulated transcriptional control. Primary events in the process of carcinogenesis can involve the activation of oncogene function by some means (e.g., amplification, mutation, chromosomal rearrangement) or altered or aberrant expression of transcriptional regulator functions, and in many cases the removal of anti-oncogene function. One reason for the enhanced growth and invasive properties of some tumors may be the acquisition of increasing numbers of mutations in oncogenes and anti-oncogenes, with cumulative effect (Bear et al., Proc. Natl. Acad. Sci. USA 86:7495-7499, 1989). Alternatively, insofar as oncogenes function through the normal cellular signalling pathways required for organismal growth and cellular function (reviewed in McCormick, Nature 363:15-16, 1993), additional events corresponding to mutations or deregulation in the oncogenic signalling pathways may also contribute to tumor malignancy (Gilks et al., Mol. Cell. Biol. 13:1759-1768, 1993), even though mutations in the signalling pathways alone may not cause cancer.

A variety of molecular targets exist for the development of efficacious anti-cancer agents, these include, without limitation, 5 alpha reductase, A-myb, ATF-3, B-myb, β-amyloid precursor protein, BSAP (also known as (Pax5), C/EBP, c-fos, c-jun, c-myb, c-myc, CDK-1 (also known as p34, cdc2), CDK-2, CDK-3, CDK-4, CDK-4 inhibitor (Arf), cHF.10 (also known of ZNF35, HF 10), COX-2, CREB, CREBP1 (also known as ATF-2), Cyclins A, B, D1, D2, D3, DB-1 (also known as ZNF161, VEZF1), DP-1, E12, E2A, E2F-1 (RBAP-1) E2F-2, E47, ELK-1, Epidermal Growth Factor Receptor, ERM, (ETV5), estrogen receptor, ERG-1, ERK-1, ERK3, ERK subunit A, ERK subunit B, Ets-1, Ets-2, FAS/APO-1, FLT-1 also known as VEGFR-1), FLT-4 (also known as VEGFR-3), Fra-1, Fra-2, GADD-45, GATA-2, GATA-3, GATA-4, HB9 (also known as MNX-1, HLSB9), HB24 (also known as HLX-1), h-plk (also known as ERV3), Hox1.3 (also known as HoxA5), Hox 2.3, (also known as HoxB7), Hox2.5 (also known as HoxB9), Hox4A (also known as HoxD3) Hox 4D (also known as HoxD10) Hox 7 (also known as MSX-1) HoxA1, HoxA10, HoxC6, HS1 (also known as 14-3-3 beta/alpha), HTF4a (also known as TCF12; HEB), I-Rel (also known as RelB), ICE (also known as CASP1; Caspase-1), ICH-1L (also known as CASP2L; Caspase-2L), ICH-1S (also known as CASP2S; Caspase-2S), ID-1, ID-2, ID-3, IRF-1, IRF-2, ISGF3, (also known as Stat1), junB, junD, KDR/FLK-1, (also known as VEGFR-2), L-myc, Ly1-1, MAD-1 (also known as MXD-1; MAD), MAD-3 (also known as NFkB1A, NFKB1, IKBA IkappaBalpha), MADS/MEF-2 (also known as MEF-2C), MAX, Mcl-1, MDR-1, MRP, MSX-2, mts1 (also known as S00A4), MXi1, MZF-1, NET (also known as ELK3; ERP), NF-IL6 (also known as C/EBPbeta; (also known as CEBPB), NF-IL6 beta (also known as C/EBPdelta, CEBPD), NF-kappa B (including 51 kD, 65kD and A subunits and intron 15), N-myc, OCT-1 (also known as POU2F1, NF-AI; OTF-1), OCT-2, OCT-3, Oct-T1, OCT-T2, OTF-3C, OZF, p53, p107, PDEGF, PDGFR, PES, Pim-1, PKC-alpha, PKC-beta, PKC-delta, PKC-epsilon, PKC-iota, Ref-1, REL (c-Rel), SAP-1, SCL (Also known as AL-1, TCL5, Stem cell protein), SGP-2 (Also known as clusterin, CLU, TRPM-2, Apolipoprotein J; APOJ, Complement associated protein SP 40,40, Complement cytolysis inhibitor, KUB1; CL1, testosterone-repressed prostate message 2), Sp-1, Sp-3, Sp-4, Spi-B (also known as PU.1 related), SRF, TGF-beta (also known as TGF beta 1, TGFB1 and TGFB), TR4, VEGF, Waf-1 (also known as p21, CAP20, CDKN1, CIP1, MDA6), WY-1 and YY-1. Of these the most preferred NABT target for cancer in general is p53. Most anticancer NABTs will provide a superior therapeutic effect when they are combined with one or more therapeutic agents that promote apoptosis. The latter includes but is not limited to conventional chemotherapy, radiation and biologic agent such as monoclonal antibodies and agents that manipulate hormone pathways.

The present invention provides NABTs which are effective to down-regulate expression of the gene products encoded by the aforementioned targets. In order to assess the effects of modifications of such NABTs (e.g., altered backbone configurations, addition of CPP, addition of endosomal lytic components, presence or absence of carriers), cell lines obtained from the cancers listed in Table 11 which are commercially available from the ATCC, can be incubated with the NABT(s) and their effects on target gene expression levels assessed.

Most cancers of the major organ systems can be excised and cultured in nude mice as xenografts. Additionally, most blood born cancers such as leukemias and lymphomas can be established in mice. Such mice provide superior in vivo models for studying the effects of the anti-cancer agents disclosed herein. The particular cancer types associated with the above-identified targets are provided in Table 11. Creating mice comprising such xenografts is well within the purview of the skilled artisan. Once the tumors are established, the NABTs of the invention, alone or in combination with the agents listed above, will be administered and the effects on reduction of tumor burden, tumor cell morphology, tumor invasive properties, angiogenesis, apoptosis, metastasis, morbidity and mortality will be determined. Alterations to NABT structures can then be assessed to find the most potent forms having efficacy for the treatment of cancer.

### B. NABTs and Methods of Use Thereof for the Treatment of Hyperproliferative Disorders.

Several hyperproliferative disorders are amenable to treatment with the NABTs described herein. Such disorders include dysplasias (e.g., cervical displasia), psoriasis, benign prostatic hyperplasia, pulmonary fibrosis, myelodysplasias, and ectodermal dysplasia. Table 11 lists targets for the NABTs associated with these disorders. These include, without limitation, 5-alpha reductase, cyclin A, cyclin B, FLT-1, Fra-2, ICE, ID-1, IRF-1, ISGF3, junB, MAD-3, p53, PDEGFR, TGF-β, TNF-α, and VEGF.

Eferl et al. report that ectopic expression of Fra-2 in transgenic mice in various organs results in generalized fibrosis with predominant manifestation in the lung (Proc Natl Acad Sci 2008 Jul. 29; 105(30):10525-30). The pulmonary phenotype was characterized by vascular remodeling and obliteration of pulmonary arteries, which coincided with expression of osteopontin, an AP-1 target gene involved in vascular remodeling and fibrogenesis. These alterations were followed by inflammation; release of profibrogenic factors, such as IL-4, insulin-like growth factor 1, and CXCL5; progressive fibrosis; and premature mortality. Genetic experiments and bone marrow reconstitutions suggested that fibrosis developed independently of B and T cells and was not mediated by autoimmunity despite the marked inflammation observed in transgenic lungs. Importantly, strong expression of Fra-2 was also observed in human samples of idiopathic and autoimmune-mediated pulmonary fibrosis. These findings indicate that Fra-2 expression is sufficient to cause pulmonary fibrosis in mice, possibly by linking vascular remodeling and fibrogenesis, and indicate that Fra-2 is a contributing pathogenic factor of pulmonary fibrosis in humans. In this embodiment of the invention, it is desirable to deliver the NABTs in an aerosolized formulation as discussed above. Other molecules which are associated with a pathological role in pulmonary fibrosis include PDEGF, PDGFR, and SRF. NABTs which effectively down modulate these targets are provided herein and should demonstrate efficacy for the treatment of pulmonary fibrosis.

Psoriasis is a chronic disease of unsolved pathogenesis affecting between one and three percent of the general population. It is characterized by inflamed, scaly and frequently disfiguring skin lesions and often accompanied by arthritis of the small joints of the hands and feet.

Haider et al. have observed increased junB mRNA and protein expression in psoriasis vulgaris lesions. See J. of Investigative Dermatology (2006) 126:912-914. Accordingly, topical administration of NABTs which down modulate expression of junB should have efficacy for the treatment of psoriasis.

In their article entitled, “Fas Pulls the Trigger on Psoriasis”, Gilhar et al. describe an animal model of psoriasis and the role played by Fas mediated signal transduction (2006) Am. J. Pathology 168:170-175). Fas/FasL signaling is best known for induction of apoptosis. However, there is an alternate pathway of Fas signaling that induces inflammatory cytokines, particularly tumor necrosis factor alpha (TNF-α) and interleukin-8 (IL-8). This pathway is prominent in cells that express high levels of anti-apoptotic molecules such as Bcl-xL. Because TNF-α is central to the pathogenesis of psoriasis and psoriatic epidermis has a low apoptotic index with high expression of Bcl-xL, these authors hypothesized that inflammatory Fas signaling mediates induction of psoriasis by activated lymphocytes. Noninvolved skin from psoriasis patients was grafted to beige-severe combined immunodeficiency mice, and psoriasis was induced by injection of FasL-positive autologous natural killer cells that were activated by IL-2. Induction of psoriasis was inhibited by injection of a blocking anti-Fas (ZB4) or anti-FasL (4A5) antibody on days 3 and 10 after natural killer cell injection. Anti-Fas monoclonal antibody significantly reduced cell proliferation (Ki-67) and epidermal thickness, with inhibition of epidermal expression of TNF-α, IL-15, HLA-DR, and ICAM-1. Fas/FasL signaling is an essential early event in the induction of psoriasis by activated lymphocytes and is necessary for induction of key inflammatory cytokines including TNF-α and IL-15.

Such data provide the rationale for therapeutic regimens entailing topical administration of NABTs targeting Fas and/or BCL-xL for the treatment and alleviation of symptoms associated with psoriasis.

p53 protein is an important transcription factor which plays a central role in cell cycle regulation mechanisms and cell proliferation control. Baran et al. performed studies to identify the expression and localization of p53 protein in lesional and non-lesional skin samples taken from psoriatic patients in comparison with healthy controls (Acta Dermatovenerol Alp Panonica Adriat. (2005) 14:79-83). Sections of psoriatic lesional and non-lesional skin (n=18) were examined. A control group (n=10) of healthy volunteers with no personal and family history of psoriasis was also examined. The expression of p53 was demonstrated using the avidin-biotin complex immunoperoxidase method and the monoclonal antibody DO7. The count and localization of cells with stained nuclei was evaluated using a light microscope in 10 fields for every skin biopsy. In lesional psoriatic skin, the count of p53 positive cells was significantly higher than in the skin samples taken from healthy individuals (p<0.01) and non-lesional skin taken from psoriatic patients (p=0.02). No significant difference between non-lesional psoriatic skin and normal skin was observed (p=0.1). A strong positive correlation between mean count and mean percent of p53 positive cells was found (p<0.0001). p53 positive cells were located most commonly in the basal layer of the epidermis of both healthy skin and non-lesional psoriatic skin. In lesional psoriatic skin p53 positive cells were present in all layers of the epidermis. In view of these data, it is clear that p53 protein appears to be an important factor in the pathogenesis of psoriasis. Accordingly, NABTs which effectively down regulate p53 expression in the skin used alone or in combination with other agents used to treat psoriasis should alleviate the symptoms of this painful and unsightly disorder.

Additional molecules which demonstrate dysregulated or overexpression in psoriatic lesions include for example, cyclins, FLT-1, ICE, ID-1, ISGSF3, and Sp-1. NABTs which effectively down modulate the expression of these targets are also provided in the present invention for use in methods for the treatment and prevention of psoriasis.

Muto et al. described newly established cervical dysplasia-derived cell lines which may be used to advantage for assessing the effects of the NABTs described herein on cervical multi-step carcinogenesis. NABTs can be added to the culture medium for human cervical dysplasia cell lines, CICCN-2 from cervical intraepithelial neoplasia grade I (CIN I), CICCN-3 from CIN II, and CICCN-4 from CIN III, and human cervical carcinoma-derived cell lines such as CICCN-6, CICCN-18, and HeLa cells and the effects on growth retardation assessed. Chromatin condensations, morphologic evidence for apoptotic cell death, can also be determined.

Certain of the hyperproliferative diseases described in the present example can be treated using transdermal drug delivery systems. Exemplary transdermal delivery systems are described by Praunitz et al. (Nature Biotechnology 26:1261-1268. First-generation transdermal delivery systems have continued their steady increase in clinical use for delivery of small, lipophilic, low-dose drugs. Second-generation delivery systems using chemical enhancers, noncavitational ultrasound and iontophoresis have also resulted in clinical products; the ability of iontophoresis to control delivery rates in real time provides added functionality. Third-generation delivery systems target their effects to skin's barrier layer of stratum corneum using microneedles, thermal ablation, microdermabrasion, electroporation and cavitational ultrasound. Microneedles and thermal ablation are currently progressing through clinical trials for delivery of a variety of macromolecules and vaccines, such as insulin, parathyroid hormone and influenza vaccine. Using these novel second- and third-generation enhancement strategies, transdermal delivery is preferred for delivery of NABTs of the invention to patients having hyperproliferative disorders of the skin and squamous epithelium.

### Example 4

### Anti-Viral NABTs and Methods of Use Thereof for the Treatment of Viral Diseases

Certain viral diseases are amenable to treatment with the NABTs described herein. For example, eight different herpesviruses infect people. Three of them—herpes simplex virus type 1, herpes simplex virus type 2, and varicella=zoster virus—cause diseases associated with blisters on the skin or mucus membranes. Another herpesvirus, Epstein-Barr virus, causes infectious mononucleosis. Human herpesviruses 6 and 7 cause a childhood condition called roseola infantum. Human herpesvirus 8 has been implicated as a cause of cancer (Kaposi's sarcoma) in people with AIDS. All of the herpesviruses remain within its host cell typically in a dormant (latent) state. Sometimes the virus reactivates and produces further episodes of disease. Reactivation may occur rapidly or many years after the initial infection.

NABTs useful for treatment of these types of invention include USF, Spi-1, Spi-B, ATF, CREB and C/EBP families, E2F-1, YY-1, Oct-1, Ap-1, Ap-2, c-myb, NF-kappaB, CDK-1, CDK-2, CDK-3, CDK-4, Cyclin B, and WAF-1.

Human embryonic lung fibroblasts (WI-38) and primary African green monkey kidney cell monolayers (Flow Laboratories, Inc., Rockville, Md.) are suitable cell cultures for optimizing the anti-viral effects of the modified NABTs described herein. The cell lines are maintained on Eagle minimal essential medium supplemented with 2.5% fetal calf serum, 7.5% NaHCO₃, and 80 U of penicillin, 80 μg of streptomycin, 0.04 mg of kanamycin, and 2 U of mycostatin per ml. Human newborn foreskin fibroblast (HFF) monolayers, grown on 12-mm cover slips in 1-dram vials (Bartels Immunodiagnostic Supplies, Inc., Bellevue, Wash.), are similarly maintained. Cell monolayers can be inoculated with fresh or frozen clinical specimens and examined for viral antigen by direct IP staining and cytopathic effect (CPE). Specimens from both genital and nongenital sources can be tested. Specimens can either be immediately inoculated into cell culture or frozen at −70° C. for later processing.

Once the cultures are prepared, the cells will be incubated in the presence and absence of the above-identified NABTs and the effects on viral antigen production and CPE assessed.

Cytomegalovirus is a cause of serious disease in newborns and in people with a weakened immune system. It can also produce symptoms similar to infectious mononucleosis in people with a healthy immune system. NABTs directed to the following targets are useful for the treatment of CMV infection: SRF, NF-kappaB, p53, Ap-1, IE-2, C/EBP, Oct-1, Rb, CDK-1, CDK-2, CDK-3, CDK-4, and WAF-1.

Animal models for the evaluation of therapies against human cytomegalovirus (HCMV) are limited due to the species-specific replication of CMV. However, models utilizing human fetal tissues implanted into SCID mice are available. An alternative approach entails the use of a model incorporating HCMV-infected human foreskin fibroblasts (HFF) seeded onto a biodegradable gelatin matrix (Gelfoam). Infected HFFs are then implanted subcutaneously into SCID mice. Such mice can then be administered the appropriate NABTs of the invention and the effects on reduction in viral titer and/or symptoms can be determined. See Bravo et al., Antiviral Res. (2007) November; 76(2):104-10.

Many antiviral drugs are currently available which work by interfering with replication of viruses. Most drugs used to treat human immunodeficiency virus (HIV) infection work this way. Several of the NABTs of the invention target molecules required for HIV replication. These include USF, Elf-1, Ap-1, Ap-2, Ap-4, Sp-1, Sp-3, Sp-4, p53, NF-kappaB, rel, GATA-3, UBP-1, EBP-P, ISGF3, Oct-1, Oct-2, Ets-1, NF-ATC, IRF-1, CDK-1, CDK-2, CDK-3, CDK-4, and WAF-1.

A human T cell line chronically infected with HIV is provided in U.S. Pat. No. 5,459,056. Initially, cells capable of replicating or being killed by HIV will be contacted with a NABT and the effect of the therapeutic on targeted gene function and viral replication assessed. Optionally, animal models of viral infection will also be utilized to assess the modified NABT described herein for efficacy. A suitable animal model for this purpose is described in Ayash-Rashkovsky et al. These investigators report that lethally irradiated normal BALB/c mice, reconstituted with murine SCID bone marrow and engrafted with human PBMC (Trimera mice), were used to establish a novel murine model for HIV-1 infection (FASEB J 2005 July; 19(9):1149-51). The Trimera mice were successfully infected with different clades and primary isolates of T- and M-tropic HIV-1, with the infection persisting in the animals for 4-6 wk. Rapid loss of the human CD4+ T cells, decrease in CD4/CD8 ratio, and increased T cell activation accompanied the viral infection. All HIV-1 infected animals were able to generate both primary and secondary immune responses, including HIV specific human humoral and cellular responses. The NABTs of the invention targeting the molecules listed above will be administered to the mice alone and in combination with other retroviral drugs and the effects on HIV replication and cellular damage assessed.

### Example 5

### NABTs for the Treatment of Diabetes and Method of Use Thereof for the Treatment of the Same

Diabetes mellitus, often referred to simply as diabetes, is a syndrome of disordered metabolism, usually due to a combination of hereditary and environmental causes, resulting in abnormally high blood sugar levels (hyperglycemia). Blood glucose levels are controlled by a complex interaction of multiple chemicals and hormones in the body, including the hormone insulin made in the beta cells of the pancreas. Diabetes mellitus refers to the group of diseases that lead to high blood glucose levels due to defects in either insulin secretion or insulin action.

Diabetes develops due to a diminished production of insulin (in type 1) or resistance to its effects (in type 2 and gestational). See World Health Organisation Department of Noncommunicable Disease Surveillance (1999). “Definition, Diagnosis and Classification of Diabetes Mellitus and its Complications”. Both lead to hyperglycemia, which largely causes the acute signs of diabetes: excessive urine production, resulting compensatory thirst and increased fluid intake, blurred vision, unexplained weight loss, lethargy, and changes in energy metabolism.

All forms of diabetes have been treatable since insulin became medically available in 1921, but there is no cure. The injections by a syringe, insulin pump, or insulin pen deliver insulin, which is a basic treatment of type 1 diabetes. Type 2 is managed with a combination of dietary treatment, exercise, medications and insulin supplementation. However, diabetes and its treatments can cause many complications. Acute complications (hypoglycemia, ketoacidosis, or nonketotic hyperosmolar coma) may occur if the disease is not adequately controlled. Serious long-term complications include cardiovascular disease (doubled risk), chronic renal failure, retinal damage (which can lead to blindness), nerve damage (of several kinds), and microvascular damage, which may cause erectile dysfunction and poor wound healing. Poor healing of wounds, particularly of the feet, can lead to gangrene, and possibly to amputation. Adequate treatment of diabetes, including strict blood pressure control and elimination of certain lifestyle factors (such as not smoking and maintaining a healthy body weight), may improve the risk profile of most of the chronic complications.

While there are effective pharmaceutical approaches for the administration of diabetes, (e.g., insulin administration, glucagon administration or agents that alter levels of either of these two molecules such as Glucophage®, Avandia®, Actos®, Januvia® and Glucovance®), it is clear given the increased prevalence of this disease, that new efficacious agents are needed for the treatment. Suitable genetic targets for this purpose include, without limitation, NABTs directed to androgen receptor, CDK-4 inhibitor, MTS-2, and p53. Use of such NABTs with the anti-diabetic agents listed above is also within the scope of the invention.

Cells and cell lines suitable for studying the effects of the NABT and modified forms thereof on glucose metabolism and methods of use thereof for drug discovery are known in the art. Such cells and cell lines will be contacted with the NABT described herein and the effects on glucagon secretion, insulin secretion and/or beta cell apoptosis can be determined. The NABT will be tested alone and in combination of 2, 3, 4, and 5 NABTs to identify the most efficacious combination for down regulating appropriate target genes. Cells suitable for these purposes include, without limitation, INS cells (ATCC CRL 11605), PC12 cells (ATCC CRL 1721), MIN6 cells, alpha-TC6 cells and INS-1 832/13 cells (Fernandez et al., J. of Proteome Res. (2007). 7:400-411). Pancreatic islet cells can be isolated and cultured as described in Joseph, J. et al., (J. Biol. Chem. (2004) 279:51049). Diao et al. (J. Biol. Chem. (2005) 280:33487-33496), provide methodology for assessing the effects of the NABTs provided herein on glucagon secretion and insulin secretion. Park, J. et al. (J. of Bioch. and Mol. Biol. (2007) 40:1058-68) provide methodology for assessing the effect of these therapeutics on glucosamine induced beta cell apoptosis in pancreatic islet cells.

A wide variety of expression vectors are available for expression of the NABT, should that be desirable to facilitate delivery to the target cells. Expression methods are described by Sambrook et al. Molecular Cloning: A Laboratory Manual or Current Protocols in Molecular Biology 16.3-17.44 (1989).

### Example 7

### NABTs Effective for Reprogramming Normal Cells

NABTs provided herein are capable of reprogramming normal cells. This feature has many applications, including but not limited to (1) generating induced pluripotent stem cells (iPS) from various somatic starting cell types such as but not limited to brain-derived neural stem cells, keratinocytes, hair follicle stem cells, fibroblasts and hematopoietic cells; (2) maintaining and expanding embryonic stem cells (ES); and (3) directing the differentiation of iPS or ES into desired cell types such as but not limited to nerve, cardiac or islet cells. ES and iPS cells can be used for a variety of medical purposes including but not limited to tissue repair. Other examples of medical conditions that can benefit from normal cell reprogramming include but are not limited to the medical need to compensate for insufficient numbers of particular normal cell types such as lymphocytes, granulocytes or megakaryocytes such as might be required to fight an infection, to replace damaged normal tissue or to increase cell numbers in vitro or in vivo for subsequent harvesting for transplant.

Tissue culture of immortal cell strains from diseased patients is an invaluable resource for medical research but is largely limited to tumor cell lines or transformed derivatives of native tissues. See Park et al. (2008) Cell, 34:877-886. These investigators have generated induced pluripotent stem (iPS) cells from patients with a variety of genetic diseases with either Mendelian or complex inheritance. Exemplary diseases include adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), Parkinson disease (PD), Huntington disease (HD), juvenile-onset, type 1 diabetes mellitus (JDM), Down syndrome (DS)/trisomy 21, and the carrier state of Lesch-Nyhan syndrome. Such disease-specific stem cells offer an unprecedented opportunity to recapitulate both normal and pathologic human tissue formation invitro, thereby enabling disease investigation and drug development. These cells provide a unique resource for assessing the reprogramming capacity of the NABTs disclosed herein.

### Example 8

### NABTs Effective for the Treatment of Diamond Blackfan Anemia

Diamond-Blackfan anemia (DBA) is characterized by anemia (low red blood cell counts) with decreased erythroid progenitors in the bone marrow. This usually develops during the neonatal period. About 47% of affected individuals also have a variety of congenital abnormalities, including craniofacial malformations, thumb or upper limb abnormalities, cardiac defects, urogenital malformations, and cleft palate. Low birth weight and generalized growth delay are sometimes observed. DBA patients have a modest risk of developing leukemia and other malignancies.

Children with DBA fail to make red blood cells and carry mutations in one copy of any of several genes encoding ribosomal proteins, which are essential components of the protein synthesis machinery. RPS19 is the most frequently mutated RP in DBA. RPS19 deficiency impairs ribosomal biogenesis. Danilova et al. (Blood (2008) 112: 5228-37) report that rps19 deficiency in zebrafish results in hematopoietic and developmental abnormalities resembling DBA. Their data suggest that the rps19-deficient phenotype is mediated by dysregulation of deltaNp63 and p53. During gastrulation, deltaNp63 is required for specification of nonneural ectoderm and its up-regulation suppresses neural differentiation, thus contributing to brain/craniofacial defects. In rps19-deficient embryos, deltaNp63 is induced in erythroid progenitors and may contribute to blood defects. These investigators have shown that suppression of p53 and deltaNp63 alleviates the rps19-deficient phenotypes. Mutations in other ribosomal proteins, such as S8, S11, and S18, also lead to up-regulation of p53 pathway, suggesting it is a common response to ribosomal protein deficiency. These findings provide new insights into pathogenesis of DBA. Ribosomal stress syndromes represent a broader spectrum of human congenital diseases caused by genotoxic stress; therefore, imbalance of p53 family members provides new targets for therapeutics.

As mentioned herein previously, the present inventor has designed a variety of discrete NABTs which down modulate expression of p53. Such NABTs can be used to advantage to treat and ameliorate the symptoms of DBA and other disorders where ribosomal defects lead to an activation of p53 expression. The sequences of these NABTs effective to inhibit expression of p53 are provided in Table 8 along with the NABT combinations provided in Table 23. However, administration of OL(1)p53 (cenersen) (SEQ ID NO: 4) which is a phosphorothioate oligo is suitable for this purpose. The use of this sequence with a 2′ fluoro gapmer is most preferred along with the oligo combinations described in Table 23 with backbones acting via steric hindrance as described elsewhere herein. For the treatment of such disorders, it preferable to administer the NABTs of the invention systemically.

### Example 9

### NABTs Targeting SGP2 for the Treatment of Disorders Characterized by Aberrant Apoptosis

SGP2 (TRPM-2 or clusterin) is expressed in cells in multiple forms as reflected in differences in amino acid sequence and non-translated sequences that are involved in regulating expression of the corresponding protein. Andersen et al. (Mol Cell Proteomics 6: 1039, 2007) have described three variants of SGP2 encoded proteins termed CLU34 (NCBI Reference Sequence NM_{—}001831), CLU35 (NCBI Reference Sequence NM_{—}203339) and CLU36 (sequence provided in supplemental information accompanying Andersen et al.). CLU 34 and CLU35 localize to the cytoplasm and are anti-apoptotic while CLU 36 is apoptotic and concentrates in the nucleus. The SGP2 gene has a total of 9 exons. The mRNA variants described by Anderson et al. each possess different first exons. CLU 34 is the variant most commonly reported in the literature. It can be secreted by cells and has a variety of extracellular functions that include interactions with growth factor pathways, such interactions being associated with inhibition of apoptosis. Leskov et al., (J Biol Chem 278: 21055, 2003) have described yet another apoptotic form in addition to CLU36 that is derived from CLU34 by an alternative splicing mechanism that results in the deletion of exon 2. The primary translational start site for CLU34 is in its first exon while the primary start site for CLU35 is in exon 2. CLU36 has a primary start site in its first exon. Alternately spliced CLU34 has its primary translational start site in exon 3.

All three SGP2 mRNA forms described by Andersen et al. are subject to differential regulation of their expression by various cellular processes which can be altered in diseased cells. For example, patterns of expression are typically altered in cancer cells such that: expression levels of the anti-apoptotic variants are increased relative to the apoptotic variants. In prostate cancer, for example, CLU34 is repressed by androgens while CLU35 is up-regulated (Cochrane et al., J Biol Chem 282: 2278, 2007). Further, CLU35 is up-regulated in prostate cancer as it progresses to androgen independence.

Two homologs (CLI and SP-40,40) are also produced by the SGP2 gene. These are distinguished by substantial divergences in the 5′ untranslated sequence particularly those in the general boundary region between intron I and exon II. This region includes hotspot 9 of the TRPM-2 gene in Table 8 which can be targeted to differentially affect the expression of these homologs. Both of these homologs bind to complement components and inhibit complement mediated cellular lysis and are of importance in biological processes such as reproduction.

A conventional antisense oligo directed to SGP2 with the sequence (5′-CAGCAGCAGAGTC TTCATCAT-3′-SEQ ID NO: 3799) is in development as a possible therapeutic agent (Schmitz, Current Opinion Mol Ther 8: 547, 2006; US 2004/0053874; 2008/0014198; U.S. Pat. Nos. 6,383,808; 6,900,187; 7,285,541; 7,368,436; WO 02/22635; 2006/056054). The terminal four nucleosides on each end of this oligo (indicated by underlining) have 2′-O-methyoxyethyl modifications to their sugar moieties. The linkages between all 21 nucleotides are phosphorothioate and the central 13 nucleosides all have deoxyribose as the sugar. It has been shown to modestly sensitize some cancer cells, including prostate cancer cells, to radiation and chemotherapeutic agents (Schmitz, Current Opinion Mol Ther 8: 547, 2006; Zellweger et al. (J Pharm Exp Ther 298: 934, 2001 and Clin Cancer Res 8: 3276, 2002). This oligo is directed to the primary translational start site for CLU35 in exon 2, but because it has an RNase H dependent mechanism of action rather than a steric hindrance mechanism of action, it indiscriminately also down-regulates CLU34 and CLU36 because they express the same exon 2. Thus, this oligo inhibits both anti-apoptotic and apoptotic forms of SGP2. Chen et al., (Cancer Res 64: 7412, 2004) have shown that this oligo can inhibit the induction of apoptosis in some cancer cells, including those deficient in p21 (WAF-1) expression, which is highly undesirable in a potential anti-cancer agent. This feature, along with its relatively poor suppressive activity on SGP2 expression is associated with a relatively low level of therapeutic efficacy.

Table 8 provides prototype conventional antisense oligo sequences and their size variants that when combined with the preferred or most preferred backbones produce surprisingly better gapmer oligos with RNase H activity in terms of suppressing SGP2 (also listed as TRPM-2 in Table 8) expression and in producing therapeutic effects such as sensitizing cancer cells to conventional cancer treatments or protecting nerve cells from the induction of apoptosis when compared to those SGP2 targeting oligos provided in the prior art such as the one just described. Specifically, 2′-fluoro gapmers with phosphorothioate linkages are most preferred with FANA or LNA gapmers being preferred. More details on gapmer oligos suitable for use in the present invention are provided elsewhere herein.

As mentioned above, certain SGP2 variants encode anti-apoptotic proteins while other variants possess apoptotic activities. When one or the other of these activities is not selectively blocked then the activity of the NABT will depend on which activity is dominant in any given situation. Selectively blocking the anti-apoptotic activity would be appropriate for treating a disorder such as cancer while selectively blocking apoptotic activity would be appropriate for the treatment of Alzheimer's Disease, for example. Table 11 lists several medical indications where NABTs directed to SGP2 should exhibit efficacy. These indications include both those characterized by pathologic induction of apoptosis as well as those where there is a pathologic resistance to the induction of apoptosis.

SGP2 transcripts encoding anti-apoptotic proteins can be selectively targeted by NABTs using one of the following design considerations: (1) the use of (a) conventional antisense oligos that support RNase H activity, (b) expression vectors or (c) siRNA or dicer substrate guide strands where the NABT binds to a segment of exon 1 of SGP2 variant CLU34 (Hot Spot 4, SEQ ID NO: 3755, in Table 8) or to a segment of exon 1 of SGP2 variant CLU35 (Hot Spot 2, SEQ ID NO: 3766, in Table 8); or (2) the use of conventional antisense oligos with selective steric hindrance activity against primary or both primary and secondary translational start sites for SGP2 variant CLU 34 (Table 18) or with selective steric hindrance activity against primary or both primary and secondary or alternative secondary translational start sites for SGP2 variant CLU35 (Table 19). Secondary translational start sites are used by cells when the primary translational start site is blocked such as by an antisense oligo with a steric hindrance mechanism.

In addition, an NABT directed to exon 1 of SGP2 variant CLU34 may be used in combination with an NABT directed to exon 1 of SGP2 variant CLU35 to simultaneously eliminate expression of both of these anti-apoptotic variants where the NABTs involved are (a) conventional antisense oligos that support RNase H activity, (b) expression vectors or (c) siRNA or dicer substrates. For cancer treatment application such NABTs will typically be used in combination with other agents that promote apoptosis such as chemotherapy, radiation and modulators of hormone activity in the case of hormonally dependent cancers.

SGP2 transcripts encoding apoptotic protein SGP2 variant CLU36 can be selectively targeted by NABTs using one of the following design considerations: (1) the use of conventional antisense oligos that support RNase H activity, expression vectors or guide strands that bind to exon 1 of SGP2 variant CLU 36 (Table 8, Hot Spot 3, SEQ ID NO: 3781); or (2) the use of conventional antisense oligos with selective steric hindrance activity against the primary and its secondary translational start site (Table 20) or the alternative primary and its secondary translational start site (Table 21).

SGP2 transcripts encoding apoptotic protein that is produced by the removal of exon 2 by alternative splicing of CLU34 can be selectively targeted by NABTs by the use of conventional antisense oligos with selective steric hindrance activity against primary or both primary and secondary translational start sites in exon 3 (Table 22).

Table 8 provides for each hot spot (presented as an antisense sequence) at least one prototype conventional antisense or prototype RNAi oligo sequence along with a listing of size variant oligo sequences that are suitable for use in NABTs in accordance with the present invention. Interpretation of the information set forth in Table 8 has been provided hereinabove.

The use of particular primary or secondary start sites, where they occur on a tissue specific basis, can be readily determined using monoclonal antibodies directed to protein sequences that would appear upstream or downstream of particular translational start sites to determine whether or not the start site is being utilized. If it is used the upstream sequence will not be seen in a Western or similar blot or other appropriate assay method and the downstream sequence will be seen. If it is not used both protein sequences will be recognized.

As for other gapmer containing conventional antisense oligos provided by the present invention, those comprising 2′-fluoro substituted sugar analogs in the terminal 5′ and 3′ nucleotides and phosphorothioate linkages between all the nucleotides are most preferred as described more fully elsewhere herein. For conventional antisense oligos with an exclusively steric hindrance mechanism of action, 2′-fluoro substituted sugar analogs for all the nucleotides coupled with phosphorothioate linkages are most preferred. Preferred chemistries are also more fully described elsewhere herein and include the following: (1) morpholino or piperazine sugar substitution in all nucleosides; (2) LNA sugar substitution in all nucleosides; and (3) FANA sugar modification in all nucleosides.

NABTs which block the anti-apoptotic effects of SGP2 variants are particularly desirable for the treatment of prostate cancer. Such NABTs can be administered systemically or directly injected into the tumor. They can be used in combination with chemotherapy, biotherapy or radiation considered appropriate for the cancer. The treatment regimens set forth above may also comprise administration of chemotherapeutic agents such as abarelix, abiraterone acetate and Degarelix.

The following tables are provided to facilitate the practice of the present invention.

Lengthy table referenced here
US20120156138A1-20120621-T00001
Please refer to the end of the specification for access instructions.

Lengthy table referenced here
US20120156138A1-20120621-T00002
Please refer to the end of the specification for access instructions.

Lengthy table referenced here
US20120156138A1-20120621-T00003
Please refer to the end of the specification for access instructions.

Lengthy table referenced here
US20120156138A1-20120621-T00004
Please refer to the end of the specification for access instructions.

Lengthy table referenced here
US20120156138A1-20120621-T00005
Please refer to the end of the specification for access instructions.

Lengthy table referenced here
US20120156138A1-20120621-T00006
Please refer to the end of the specification for access instructions.

Lengthy table referenced here
US20120156138A1-20120621-T00007
Please refer to the end of the specification for access instructions.

Lengthy table referenced here
US20120156138A1-20120621-T00008
Please refer to the end of the specification for access instructions.

Lengthy table referenced here
US20120156138A1-20120621-T00009
Please refer to the end of the specification for access instructions.

Lengthy table referenced here
US20120156138A1-20120621-T00010
Please refer to the end of the specification for access instructions.

Lengthy table referenced here
US20120156138A1-20120621-T00011
Please refer to the end of the specification for access instructions.

Lengthy table referenced here
US20120156138A1-20120621-T00012
Please refer to the end of the specification for access instructions.

Lengthy table referenced here
US20120156138A1-20120621-T00013
Please refer to the end of the specification for access instructions.

Lengthy table referenced here
US20120156138A1-20120621-T00014
Please refer to the end of the specification for access instructions.

Lengthy table referenced here
US20120156138A1-20120621-T00015
Please refer to the end of the specification for access instructions.

Lengthy table referenced here
US20120156138A1-20120621-T00016
Please refer to the end of the specification for access instructions.

Lengthy table referenced here
US20120156138A1-20120621-T00017
Please refer to the end of the specification for access instructions.

Lengthy table referenced here
US20120156138A1-20120621-T00018
Please refer to the end of the specification for access instructions.

Lengthy table referenced here
US20120156138A1-20120621-T00019
Please refer to the end of the specification for access instructions.

Lengthy table referenced here
US20120156138A1-20120621-T00020
Please refer to the end of the specification for access instructions.

Lengthy table referenced here
US20120156138A1-20120621-T00021
Please refer to the end of the specification for access instructions.

Lengthy table referenced here
US20120156138A1-20120621-T00022
Please refer to the end of the specification for access instructions.

Lengthy table referenced here
US20120156138A1-20120621-T00023
Please refer to the end of the specification for access instructions.

While certain preferred embodiments of the present invention have been described and specifically exemplified above, it is not intended that the invention be limited to such embodiments. Various modifications may be made to the invention without departing from the scope and spirit thereof as set forth in the following claims.

LENGTHY TABLES
The patent application contains a lengthy table section. A copy of the table is available in electronic form from the USPTO web site (http://seqdata.uspto.gov/?pageRequest=docDetail&DocID=US20120156138A1). An electronic copy of the table will also be available from the USPTO upon request and payment of the fee set forth in 37 CFR 1.19(b)(3).

What is claimed is:

1. A composition, comprising in a biologically acceptable carrier, at least one nucleic acid based therapeutic (NABT) for down modulating target gene expression, said NABT comprising a nucleic acid sequence which inhibits production of at least one gene product encoded by said target gene, said sequence optionally comprising one or more modifications selected from the group consisting of
i) at least one modification to the phosphodiester backbone linkage;
ii) at least one modification to a sugar in said nucleic acid;
iii) a support;
iv) at least one cellular penetrating peptide or a cellular penetrating peptide mimetic;
v) an endosomal lytic moiety;
vi) at least one specific binding pair member or targeting moiety; and
viii) operable linkage to an expression vector,
wherein said nucleic acid sequence is selected from the group of sequences in Table 8, with the proviso that when i, ii, iii, iv, v, vi, viii are absent, said nucleic acid is not SEQ ID NOS: 1, 2, 3, 4, or 2265-2293.

2. The composition of claim 1, wherein said nucleic acid comprises at least one modified linkage selected from the group consisting of phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2′-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages.

3. The composition of claim 1 or 2, which comprises at least one modified sugar selected from the group consisting of 2′ fluoro, 2′ fluoro substituted ribose, 2-fluoro-D-arabinonucleic acid, 2′-O-methoxyethyl ribose, 2′-O-methoxyethyl deoxyribose, 2′-O-methyl substituted ribose, a morpholino, a piperazine, and a locked nucleic acid.

4. The composition of claim 1, 2 or 3 wherein said nucleic acid is a conventional antisense nucleic acid which functions via a steric hindrance mechanism.

5. The composition of claim 1 or 2, or 3, wherein said nucleic acid is a modified antisense nucleic acid which functions by triggering RNAse H activity.

6. The composition of claim 5, wherein said nucleic acid is a gapmer which promotes RNAse H activity and exhibits increased binding affinity for said target nucleic acid.

7. The composition of claim 1, wherein said nucleic acid is an RNAi.

8. The composition of claim 1 or 2, or 7 wherein said nucleic acid sequence is operably linked to an expression vector which produces an NABT which inhibit expression of said target gene upon introduction of said vector into a cell.

9. The composition of claim 5 or 6, comprising a modification selected from the group consisting of a LNA modification, a FANA modification, a 2′ fluoro substituted ribose, at least one morpholino, or at least one piperazine, wherein NABT is a 14-22mer with phosphorothioate linkages and a 4-18 nucleoside core comprising deoxyribose or a functional analog thereof.

10. The composition of claim 9, wherein said gapmer comprises at least one base modification selected from the group consisting of 4′-C-hydroxymethyl-DNA, 3′-C-hydroxymethyl-arabinonucleic acid, piperazino-functionalized C3′,02′-linked arabinonucleic acid, wherein said modified base is inserted near the center of the NABT within 4 nucleosides of either the 5′ or 3′ end of said NABT.

11. The composition of claim 9 or 10 comprising at least one modified nucleotide selected from the group consisting of 2′ fluoro-arabinonucleotides, abasic nucleotides, tetrahydrofurans (THF), bases shown in Formulas I, II and III wherein each of _{R1-8} is independently selected from H, halogen, and C₁₋₃ alkyl, R₈ may also be independently selected from fluorine and methyl, and bases selected from Formulas IV-XII.

12. The composition of claim 1 to claim 11, comprising a support selected from the group consisting of nanoparticles, dendrimers, nanocapsules, nanolattices, microparticles, micelles, Hemagglutinating virus of Japan (HVJ) envelope, spiegelmers, and liposomes.

13. The composition of claim 1 to claim 12 wherein said NABT is operably linked to a cellular penetrating peptide or mimetic thereof selected from the group consisting of one or more of
(SEQ ID NO: 3631)
KRRQRRR;
(SEQ ID NO: 3632)
GYGRKKRRQRRR;
(SEQ ID NO: 3633)
YGRKKRRQRRR;
(SEQ ID NO: 3634)
CYGRKKRRQRRR;
(SEQ ID NO: 3635)
RKKRRQRRRPPQC;
(SEQ ID NO: 3636)
CYQRKKRRQRRR;
(SEQ ID NO: 3637)
RKKRRQRRR;
(SEQ ID NO: 3638)
GALFLGF(or W)LGAAGSTMGA;
(SEQ ID NO: 3639)
GALFLGF(or W)LGAAGSTMGAWSQPKKKRKV;
(SEQ ID NO: 3640)
GALFLGF(or W)LGAAGSTMGAWSQPKSKRKV;;
(SEQ ID NO: 3641)
RQIKIWFQNRRMKWKK;
(SEQ ID NO: 3642)
RQIKIWFQNRRMKWKKGGC;
(SEQ ID NO: 3643)
LIRLWSHLIHIWFQNRRLKWKKK;
(SEQ ID NO: 3644)
GLFGAIAGFIENGWEGMIDGRQIKIWFQNRRMKWKK;
SEQ ID NO: 3645)
FFGAVIGTIALGVATA;
(SEQ ID NO: 3646)
FLGFLLGVGSAIASGV;
(SEQ ID NO: 3647)
GVFVLGFLGFLATAGS;
(SEQ ID NO: 3648)
GAAIGLAWIPYFGPAA;
(SEQ ID NO: 3649)
DAATATRGRSAASRPTERPRAPARSASRPRRPVD (or E);
(SEQ ID NO: 3650)
KLAKLLALKALKAALKLA;
(SEQ ID NO: 3651)
KLALKLALKALKAALKLA;
(SEQ ID NO: 3652)
KETWWETWWTEWSQPKKKRKV;
(SEQ ID NO: 3653)
KETWFETWFTEWSQPKKKRKV;
(SEQ ID NO: 3654)
KXaaXaaWWETWWXaaXaaXaaSQPKKXaaRKXaa;
(SEQ ID NO: 3655)
KETWWETWWTEWSQPKKRKV;
(SEQ ID NO: 3656)
KETWWETWWTEASQPKKRKV;
(SEQ ID NO: 3657)
KETWWETWWETWSQPKKKRKV;
(SEQ ID NO: 3658)
KETWWETWTWSQPKKKRKV;
(SEQ ID NO: 3659)
KWWETWWETWSQPKKKRKV;
(SEQ ID NO: 3660)
KETWWETWWXaaXaaWSQPKKKRKV;
(SEQ ID NO: 3661)
GALFLGWLGAAGSTM;
(SEQ ID NO: 3662)
GALFLGWLGAAGSTMGAWSQPKKKRKV;
(SEQ ID NO: 3663)
MVKSKIGSWILVLFVAMWSDVGLCKKRPKP;
(SEQ ID NO: 3664)
RGGRLSYSRRRFSTSTGR;;
(SEQ ID NO: 3665)
RRLSYSRRRF;;
(SEQ ID NO: 3666)
GWILNSAGYLLGKINLKALAALAKKIL;
(SEQ ID NO: 3667)
AGYLLGKINLKALAALAKKIL;
(SEQ ID NO: 3668)
R6WGR6-PKKKRKV;
(SEQ ID NO: 3669)
R4SR6FGR-6VWR4-PKKKRKV;
(SEQ ID NO: 3677)
S413PV;
(SEQ ID NO: 3678)
SAP;
(SEQ ID NO: 3680)
ARF based CPP;
(SEQ ID NO: 3681)
ARF based CPP;
(SEQ ID NO: 3682)
ARF based CPP;
(SEQ ID NO: 3691)
Anti-microbial peptide;
(SEQ ID NO: 3692)
Anti-microbial peptide;
(SEQ ID NO: 3693)
Anti-microbial peptide;
(SEQ ID NO: 3694)
Anti-microbial peptide;
(SEQ ID NO: 3695)
Anti-microbial peptide;
(SEQ ID NOS: 3696-3713, 3800 and 3801)
Designer CPPs;
and
(SEQ ID NO: 3697)
Designer CPP.

14. The composition of claim 1 to claim 13, comprising an endosomal lytic component.

15. The composition of claim 1 to claim 14 comprising at least one member of a specific binding pair or targeting moiety.

16. The composition of claim 15 wherein said binding pair member or targeting moiety is selected from the group consisting of ligands for leptin receptor, ligands for lipoprotein receptor, peptides that target the LOX-1 receptor, LFA-1 targeting moieties, NL4-10K, IFG-1 targeting peptides, ligands for the transferrin receptor, ligands for transmembrane domain protein 30A, ligands for asialoglycoprotein receptor, Trk targeting ligands, an actively transported nutrient, RVG peptide, heart homing peptides, peptide for ocular delivery, and PH-50.

17. The composition of claim 1 to claim 16, operably linked to an expression vector, said vector facilitating cellular uptake and expression of said NABT encoding sequences within the cell resulting in down modulation of the sequence targeted by said NABT.

18. The composition as claimed in claim 7 or 16, wherein said NABT is a double stranded dicer substrate RNA comprising a passenger strand and a guide strand 25-30-nucleotides in length which is cleaved intracellularly to form substantially double stranded 21-mers with a two nucleotide (2-nt) overhang on each 3′ end.

19. The composition of claim 18, wherein the 5′ end of a passenger strand RNA is blocked with an alkyl group, thereby increasing guide strand loading into the RISC complex.

20. The composition of claim 19, wherein said passenger strand is nicked or comprises a gap.

21. The composition of claim 18, wherein a 5′ end of the passenger strand is modified at 1, 2, 3 or 4 positions, thereby increasing Tm of duplex formation with a corresponding guide strand.

22. The composition of claim 18, wherein the affinity of the four nucleotides at the 3′ end of the passenger stand for the 5′ end of the guide strand is decreased relative to the opposite end of the duplex.

23. A formulation, comprising the composition of claim 1 to claim 22, suitable for systemic, aerosolized, oral and topical formulations.

24. The formulation of claim 23, selected from the group consisting of oral, intrabuccal, intrapulmonary, rectal, intrauterine, intratumor, intracranial, nasal, intramuscular, subcutaneous, intravascular, intrathecal, inhalable, transdermal, intradermal, intracavitary, implantable, iontophoretic, ocular, vaginal, intraarticular, otical, intravenous, intramuscular, intraglandular, intraorgan, intralymphatic, implantable, slow release, and enteric coating formulations.

25. A method for down modulating expression of a target gene for the treatment of an aberrant programming disease in a target cell, said method comprising administration of an effective amount of at least one composition comprising an NABT as claimed in any one of the preceding claims, thereby reprogramming said target cell, said reprogramming altering the aberrant programming disease phenotype thereby providing a beneficial therapeutic or commercial effect.

26. The method of claim 25, wherein said NABT down modulates expression of a transcriptional regulator.

27. The method of claim 25, wherein said NABT down modulates expression of a direct modifier of a transcriptional regulator.

28. The method of claim 25, wherein said reprogramming is therapeutically beneficial to diseased cells and normal cells are not adversely affected.

29. The method of claim 25 to claim 28, wherein said cell is in a patient.

30. The method of claim 25 to claim 29, further comprising administration of an augmentation agent, selected from the group consisting of antioxidants, polyunsaturated fatty acids, chemotherapeutic agents, genome damaging agents and ionizing radiation.

31. A method as claimed in claim 25 to claim 30, wherein said disease is selected from the group consisting of Cancer, AIDS, Alzheimer's disease, Amyotrophic lateral sclerosis, Atherosclerosis, Autoimmune Diseases, Cerebellar degeneration, Cancer, Diabetes Mellitus, Glomerulonephritis, Heart Failure, Macular Degeneration, Multiple sclerosis, Myelodysplastic syndromes, Parkinson's disease, Prostatic hyperplasia, Psoriasis, Asthma, Retinal Degeneration, Retinitis pigmentosa, Rheumatoid arthritis, Rupture of atherosclerotic plaques, Systemic lupus erythematosis, Ulcerative colitis, viral infection, ischemia reperfusion injury, cardiohypertrophy, and Diamond Black Fan anemia.

32. The method as claimed in claim 31, wherein said disease is a viral disease and said NABT is effective to reduce viral replication, load or spread.

33. The method as claimed in claim 32, wherein said viral disease is HIV and said target is selected from the group consisting of at least one of USF, Ap-2, Ap-4, Sp-1, Sp-3, Sp-4, p53, NF-κβ, and C/EBP.

34. An anti-viral composition effective against HIV for use in the method of claim 32, comprising at least one NABT having a sequence selected from the group consisting of USF (SEQ ID NOS: 3484-3508), Ap-2 (SEQ ID NOS: 48-84), Ap-4 (SEQ ID NOS: 85-107), Sp-1 (SEQ ID NOS: 3198-3208), Sp-3 (SEQ ID NOS: 3209-3212), Sp-4 (SEQ ID NOS: 3213-3219), p53 (SEQ ID NOS:4, 2806-2815, 3606-3626, and 3786-3798), (NF-κβ SEQ ID NOS: 2524-2620), and C/EBP (SEQ ID NOS: 336-345) in pharmaceutically acceptable carrier.

35. The method as claimed in claim 32, wherein said viral disease is CMV and said target is selected from the group consisting of at least one of SRF, NF-κβ, p53, and C/EBP.

36. An anti-viral composition effective against CMV for use in the method of claim 35, comprising an effective amount of at least one NABT having a sequence selected from the group consisting of at least one of SRF (SEQ ID NOS: 3260-3290), NF-κβ (SEQ ID NOS: 2524-2620), p53 (SEQ ID NOS:4, 2806-2815, 3606-3626, and 3786-3798), and C/EBP (SEQ ID NOS: 336-345) in a pharmaceutically acceptable carrier.

37. The method as claimed in claim 32, wherein said viral disease is herpesvirus and said target is USF, Spi-1, Spi-B, ATF, CREB, C/EBP, E2F, YY-1, Oct-1, Ap-1, Ap-2, c-myb, and NF-κβ.

38. An anti-viral composition effective against herpes virus infection for use in the method of claim 37, comprising an effective amount of at least one NABT having a sequence selected from the group consisting of USF (SEQ ID NOS: 3484-3508), Spi-1 (SEQ ID NOS: 3220-3240), Spi-B (SEQ ID NOS: 3241-3259), ATF (SEQ ID NOS: 194-205), CREB (SEQ ID NOS: 515-577), C/EBP (SEQ ID NOS: 336-345), E2F (SEQ ID NOS: 846-888), YY-1 (SEQ ID NOS: 3596-3601), Oct-1 (SEQ ID NOS: 2631-2653), Ap-2 (SEQ ID NOS: 48-84), c-myb (SEQ ID NOS: 382-387), and NF-κβ (SEQ ID NOS: 2524-2620) in a pharmaceutically acceptable carrier suitable for topical administration.

39. The method as claimed in claim 32, wherein said viral disease is hepatitis virus and said target is NF-1, Ap-1, Sp-1, RFX-1, RFX-2, RFX-3, NF-κβ, Ap-2 and C/EBP.

40. An anti-viral composition effective against hepatitis virus for use in the method of claim 39, comprising an effective amount of at least one NABT having a sequence selected from the group consisting of Sp-1 (SEQ ID NOS 3198-3208), NF-κβ (SEQ ID NOS: 2524-2620), Ap-2 (SEQ ID NOS: 48-84) and C/EBP (SEQ ID NOS: 336-345).

41. The method as claimed in claim 31, wherein said disease in heart failure and said target is selected from the group consisting of p53, BCL-X, Bcl-2-like 1, BCL2L1, BCL2L, Bcl-xS, FAS/APO1, Pro-apoptotic form of gene product, DB-1, (ZNF161; VEZF1), ICE (CASP1; Caspase-1), NF-kappaB, PKC alpha, SRF and VEGF, said NABT optionally being linked to a heart homing peptide.

42. A composition useful for the treatment of heart failure for use in the method of claim 41, comprising an effective amount of at least one NABT having a sequence selected from the group consisting of those targeting p53, BCL-X, Bcl-2-like 1, BCL2L1, BCL2L, Bcl-xS, FAS/APO 1, Pro-apoptotic form of gene product, DB-1, (ZNF161; VEZF1), ICE (CASP1; Caspase-1), NF-kappaB, PKC alpha, SRF and VEGF, said NABT optionally being operably linked to a heart homing peptide in a pharmaceutically acceptable carrier.

43. The composition of claim 42, comprising a heart homing peptide of SEQ ID NOS 3715-3719.

44. The method as claimed in claim 31, wherein said disease is cancer and said sequence targeted by said NABT is selected from the group consisting of at least one of 5 alpha reductase, A-myb, ATF-3, B-myb, β-amyloid precursor protein, BSAP, C/EBP, c-fos, c-jun, c-myb, c-myc, CDK-1, CDK-2, CDK-3, CDK-4, CDK-4 inhibitor (Arf), cHF.10, COX-2, CREB, CREBP1, Cyclins A, B, D1, D2, D3, DB-1, DP-1, E12, E2A, E2F-1, E2F-2, E47, ELK-1, Epidermal Growth Factor Receptor, ERM, (ETV5), estrogen receptor, ERG-1, ERK-1, ERK3, ERK subunit A, ERK subunit B, Ets-1, Ets-2, FAS/APO-1, FLT-1, FLT-4, Fra-1, Fra-2, GADD-45, GATA-2, GATA-3, GATA-4, HB9, HB24, h-plk, Hox1.3, Hox 2.3, Hox2.5, Hox4A, Hox 4D, Hox 7, HoxA1, HoxA10, HoxC6, HS1, HTF4a, I-Rel, ICE, ICH-1L, ICH-1S, ID-1, ID-2, ID-3, IRF-1, IRF-2, ISGF3, junB, junD, KDR/FLK-1, L-myc, Lyl-1, MAD-1, MAD-3, MADS/MEF-2, MAX, Mcl-1, MDR-1, MRP, MSX-2, mts1, MXi1, MZF-1, NET, NF-IL6, C/EBPbeta, NF-IL6 beta, NF-kappa B, N-myc, OCT-1, OCT-2, OCT-3, Oct-T1, OCT-T2, OTF-3C, OZF, p53, p107, PDEGF, PDGFR, PES, Pim-1, PKC-alpha, PKC-beta, PKC-delta, PKC-epsilon, PKC-iota, Ref-1, REL, SAP-1, SCL, SGP-2, TRPM-2 Apolipoprotein J; APOJ, Complement associated protein SP 40,40, Complement cytolysis inhibitor, KUB1; CL1, testosterone-repressed prostate message 2), Sp-1, Sp-3, Sp-4, Spi-B, SRF, TGF-beta, TR4, VEGF, Waf-1, WY-1 and YY-1, said method optionally comprising administration of an at least one augmention agent, chemotherapeutic, biologic or anti-proliferative agent.

45. The method as claimed in claim 44, wherein said cancer is selected from the group consisting of brain cancer, lung cancer, ovarian cancer, breast cancer, testicular cancer, kidney cancer, liver cancer, skin cancer, pancreatic cancer, esophageal cancer, stomach cancer, bladder cancer, uterine cancer, prostate cancer, glaucomas, sarcomas, myelomas, lymphomas, and leukemias.

46. The method of claim 44, wherein said agent is selected from the group consisting of at least one of a toxin, saporin, ricin, abrin, ethidium bromide, diptheria toxin, Pseudomonas exotoxin, an alkylating agent, a nitrogen mustards, chlorambucil, cyclophosphamide, isofamide, mechlorethamine, melphalan, uracil mustard; aziridines, thiotepa; a methanesulphonate ester, busulfan; carmustine, lomustine, streptozocin; cisplatin, carboplatin; mitomycin, procarbazine, dacarbazine and altretamine, bleomycin, amsacrine, dactinomycin, daunorubicin, idarubicin, mitoxantrone, doxorubicin, etoposide, teniposide, plicamydin, methotrexate, trimetrexate; fluorouracil, fluorodeoxyuridine, CB3717, azacitidine, cytarabine, floxuridine; mercaptopurine, 6-thioguanine, fludarabine, pentostatin; asparginase, hydroxyurea, vincristine, vinblastine, paclitaxel (Taxol), estrogens; conjugated estrogens; ethinyl estradiol; diethylstilbesterol; chlortrianisen; idenestrol; hydroxyprogesterone caproate, medroxyprogesterone, megestrol; testosterone, testosterone propionate, fluoxymesterone, methyltestosterone, abarelix abiraterone acetate, Degarelix, prednisone, dexamethasone, methylprednisolone, and prednisolone, leuprolide acetate, goserelin acetate, tamoxifen, flutamide, mitotane, and aminoglutethimide.

47. The method of claim 46 wherein said chemotherapeutic agent is selected from the group consisting of: pacitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

48. The method of claim 44 to claim 47, wherein said NABT and said anti-cancer or anti-proliferative agent act synergistically.

49. The method of claim 44 to claim 47, wherein said cancer is prostate cancer, said at least one NABT is selected from the group consisting of those targeting 5 alpha-reductase, β amyloid precursor protein, cyclin A, cyclin D3, Oct-T1, p53, Pim-1, Ref-1, SAP-1, SGP2, SRF, TGF-beta, TRPM-2, clusterin and said chemotherapeutic agent is selected from the group consisting of Abarelix, abiraterone acetate, and Degarelix.

50. The method of claim 49 further comprising administration of an augmentation agent.

51. The method of claim 31, wherein said disease is Alzheimer's disease and said sequence targeted by said NABT is selected from the group consisting of apolipoprotein epsilon 4, β amyloid precursor protein, CDK-2, Cox-2, CREB, CREBP, Cyclin B, ICH-1L (also known as caspase 2L), PKC genes, PDGFR, SGP2, SRF, and TRPM-2, said NABT optionally comprising a cellular peneratrating peptide (CPP) to facilitate penetration of the blood brain barrier, thereby enhancing uptake of said NABT into cells of the CNS.

52. The method of claim 31, wherein said disease is Multiple sclerosis and said target is selected from the group consisting of p53, COX-2 TNF-α, and TNF-β and said composition is administered nasally.

53. The method of claim 31 wherein said disease is diabetes and said NABT targets a gene selected from the group consisting of androgen receptor, CDK-4 inhibitor, MTS-2, and p53.

54. The method of claim 53 further comprising administration of at least one agent selected from the group consisting of Glucophage®, Avandia®, Actos®, Januvia® and Glucovance®).

55. The method of claim 31 wherein said disease is asthma and said target is selected from the group consisting of ISGF3, PES, REF-1, and TNF-alpha.

56. The method of claim 55, further comprising administration of at least one agent selected from the group consisting of cortisone, hydrocortisone, prednisone, prednylidene, prednisolone, methylprednisolone, beclomethasone, flunisolide, triamcinolone, deflazacort, betamethasone and dexamethasone.

57. The method of claim 31, wherein said disease is atherosclerosis and said target is selected from the group consisting of at least one of DB-1, DP-1, E2F-1, ERG-1, FLT-4, ICH-1L, ISGF3, NF-IL6, OCT-1, p53, Sp-1, PDEGF, and PDGFR.

58. The method of claim 31, wherein said disease is psoriasis and said target is selected from the group consisting of at least one of Bcl-xL, cyclin A, cyclin B, Flt-1, ICE, ID-1, ISGF3, junB, p53, sp1, TNF-alpha, VEGF, and NF-kappa B and said NABT is administered topically.

59. The method of claim 31, wherein said disease is Diamond Blackfan anemia and said target is p53.

60. The method of claim 59, wherein said NABT has a sequence selected from the group consisting of at least one of SEQ ID NOS: 2806-2818, 3606-3626, 3786-3798 and modified SEQ ID NO: 4.

61. The method of claim 60, wherein SEQ ID NO: 4 comprises a 2′ fluoro gapmer which acts via a steric hindrance mechanism.

62. The method of claim 60, wherein at least two NABTs directed to p53, said pair of NABTs being selected from those in Table 23.

63. The method for the treatment of prostate cancer as claimed in claim 49 or 50 comprising administration of a pair of NABTs directed to SGP-2 or clusterin.

64. The method of claim 63, wherein said NABT directed to SGP-2 or clusterin are selected from those set forth in Tables 18-22.

65. The method as claimed in claim 31, wherein said disease is pulmonary fibrosis and said at least one NABT is aerosolized and targets a gene selected from the group consisting of Fra-2, PDEGF, PDGFR, and SRF.

66. The method as claimed in claim 31, wherein said disease is systemic lupus erythematosis and said at least one NABT targets a gene selected from the group consisting of CREM, Fas/APO-1, HS1, Oct-T1 and p53.

67. A method for optimizing the efficacy of NABT for treatment of aberrant programming diseases:
a) selecting a target gene sequence which regulates cellular programming and a sequence which hybridizes therewith from Table 8;
b) incubating the aberrantly programmed diseased cells in the presence and absence of said at least one NABT molecule, said NABT comprising one or more modifications selected from the group consisting of
i) at least one modification to the phosphodiester backbone linkage;
ii) at least one modification to a sugar in said nucleic acid;
iii) a support;
iv) at least one cellular penetrating peptide or a cellular penetrating peptide mimetic;
v) an endosomal lytic moiety;
vi) at least one specific binding pair member or targeting moiety; and
viii) operable linkage to an expression vector,
c) identifying those NABT which exhibit improved effects on cellular reprogramming relative to cells treated NABT lacking at least one modification of step b); thereby identifying efficacious modified NABT for the treatment of aberrant programming disorders.

68. The method of claim 67, comprising contacting normal cells with the NABT identified in step c) thereby identifying those NABTs which differentially affect cellular programming in aberrantly programmed cells versus normal cells.

69. The method as claimed in claim 67 or claim 68 wherein said aberrant programming disease is selected from the group consisting of AIDS, Alzheimer's disease, Amylotrophic lateral schlerosis, Atherosclerosis, restenosis, Cerebellar degeneration, cancer, Diamond Blackfan anemia, immune-mediated glomerulonephritis, toxin-induced liver disease, multiple organ dysfunction syndrome, multiple sclerosis, myelodysplastic syndrome, myocardial infarction, heart failure, psoriasis, rupture of aortic plaques, Parkinson's disease, ischemia-reperfusion injury, retinitis pigmentosa, arthritis, asthma, stroke, systemic lupus erythematosis,

70. The method of claim 67 to claim 69, wherein said disease comprises aberrant apoptosis and said NABT is directed to bcl-2α or bcl-2β.

71. The method of claim 67 to claim 70 wherein said NABT is directed to a transcriptional regulator selected from the group consisting of
p34 (cdc2), SEQ ID NOS: 944-966;
p53 (SEQ ID NOS:4, 2806-2815, 3606-3626, and 3786-3798)
fas/Apo 1, SEQ ID NOS: 3287-3293.
mts-1, SEQ ID NOS: 2454-2472;
mts-2, SEQ ID NOS: 2100-2120;
NfκB, SEQ ID NOS: 1720-1739, 1741-1774, and 2166-2205;
WAF1 (p21), SEQ ID NOS: 2440-2453;
RB, (SEQ ID NOS: 400, 402, 404, 406, 408, 410, 411, 413, 415, 417 and 419);
ref-1, (SEQ ID NOS: 2657-2678);
c-myc, (SEQ ID NOS: 657-676);
n-myc, (SEQ ID NOS: 639-648);
SGP-2, (SEQ ID NOS: 3175-3197, 3746-3785) and
TRPM-2, (SEQ ID NOS: 3419-3483.

72. The method as claimed in claim 67 to claim 71, further comprising the step of assessing the oligonucleotide so identified for efficacy and toxicity in an in vivo animal model.

73. The method as claimed in claim 72, wherein said animal model is a non-human primate model for AIDS.

74. The method as claimed in claim 67, wherein disease is cancer and said modified NABT is assessed in an immunocompromised tumor bearing animal.

75. The method as claimed in claim 74, wherein said NABT targets at least one region in the p53 gene sequence.

76. The method as claimed in claim 67, wherein said NABT is selected from the group consisting of an antisense NABT, a modified antisense NABT, an siRNA NABT, a modified siRNA NABT, a ribozyme NABT, each of the NABT optionally being encoded by an expression vector suitable for expressing said NABT in a target cell.

77. The composition as claimed in claim 1, 2, or 3 wherein said NABT acts via a steric hindrance mechanism and also triggers RNAse H activity.
